SEQUENCE LISTING (29 sequences)

SEQ ID NO: 1 - protein, 23 aa, Artificial Sequence
  Chemically synthesized RGD peptide
GRGDIPASSKGGGGSRLLLLLLR

SEQ ID NO: 2 - protein, 23 aa, Artificial Sequence
  Chemically synthesized acylated RGD peptide
GRGDIPASSKGGGGSRLLLLLLR

SEQ ID NO: 3 - protein, 10 aa, Artificial Sequence
  Chemically synthesized spacer peptide SGGGGKSSAP
SGGGGKSSAP

SEQ ID NO: 4 - protein, 6 aa, Artificial Sequence
  Chemically synthesized hydrophilic domain
KQAGDV

SEQ ID NO: 5 - protein, 5 aa, Artificial Sequence
  Chemically synthesized hydrophilic domain
PHSRN

SEQ ID NO: 6 - protein, 5 aa, Artificial Sequence
  Chemically synthesized hydrophilic domain YIGSR
YIGSR

SEQ ID NO: 7 - protein, 9 aa, Artificial Sequence
  Chemically synthesized hydrophilic domain CDPGYIGSR
CDPGYIGSR

SEQ ID NO: 8 - protein, 5 aa, Artificial Sequence
  Chemically synthesized hydrophilic domain IKVAV
IKVAV

SEQ ID NO: 9 - protein, 10 aa, Artificial Sequence
  Chemically synthesized hydrophilic domain RNIAEIIKDI
RNIAEIIKDI

SEQ ID NO: 10 - protein, 7 aa, Artificial Sequence
  Chemically synthesized hydrophilic domain YFQRYLI
YFQRYLI

SEQ ID NO: 11 - protein, 5 aa, Artificial Sequence
  Chemically synthesized hydrophilic domain PDSGR
PDSGR

SEQ ID NO: 12 - protein, 7 aa, Artificial Sequence
  Chemically synthesized hydrophilic domain FHRRIKA
FHRRIKA

SEQ ID NO: 13 - protein, 6 aa, Artificial Sequence
  Chemically synthesized hydrophilic domain PRRARV
PRRARV

SEQ ID NO: 14 - protein, 8 aa, Artificial Sequence
  Chemically synthesized hydrophilic domain WQPPRARI
WQPPRARI

SEQ ID NO: 15 - protein, 5 aa, Artificial Sequence
  Chemically synthesized hydrophobic/apolar tail LLLLL
LLLLL

SEQ ID NO: 16 - protein, 5 aa, Artificial Sequence
  Chemically synthesized hydrophobic/apolar tail LLFFL
LLFFL

SEQ ID NO: 17 - protein, 5 aa, Artificial Sequence
  Chemically synthesized hydrophobic/apolar tail LFLWL
LFLWL

SEQ ID NO: 18 - protein, 5 aa, Artificial Sequence
  Chemically synthesized hydrophobic/apolar tail FLWLL
FLWLL

SEQ ID NO: 19 - protein, 5 aa, Artificial Sequence
  Chemically synthesized hydrophobic/apolar tail LALGL
LALGL

SEQ ID NO: 20 - protein, 6 aa, Artificial Sequence
  Chemically synthesized hydrophobic/apolar tail LLLLLL
LLLLLL

SEQ ID NO: 21 - protein, 7 aa, Artificial Sequence
  Chemically synthesized hydrophobic/apolar tail RLLLLLR
RLLLLLR

SEQ ID NO: 22 - protein, 7 aa, Artificial Sequence
  Chemically synthesized hydrophobic/apolar tail KLLLLLR
KLLLLLR

SEQ ID NO: 23 - protein, 7 aa, Artificial Sequence
  Chemically synthesized hydrophobic/apolar tail KLLLLLK
KLLLLLK

SEQ ID NO: 24 - protein, 14 aa, Artificial Sequence
  Chemically synthesized spacer GSPGISGGGGGILE
GSPGISGGGGGILE

SEQ ID NO: 25 - protein, 11 aa, Artificial Sequence
  Chemically synthesized spacer SGGGGKSSAPI
SGGGGKSSAPI

SEQ ID NO: 26 - protein, 21 aa, Artificial Sequence
  Chemically synthesized spacer-tail peptide GSPGISGGGGGILEKLLLLLK
GSPGISGGGGGILEKLLLLLK

SEQ ID NO: 27 - protein, 22 aa, Artificial Sequence
  Chemically synthesized spacer-tail peptide GSPGISGGGGGILEKLALWLLR
GSPGISGGGGGILEKLALWLLR

SEQ ID NO: 28 - protein, 20 aa, Artificial Sequence
  Chemically synthesized spacer-tail peptide GSPGISGGGGGILERLLLLR
GSPGISGGGGGILERLLLLR

SEQ ID NO: 29 - protein, 21 aa, Artificial Sequence
  Chemically synthesized spacer-tail peptide GSPGISGGGGGILERLLWLLR
GSPGISGGGGGILERLLWLLR

(19) United States
(12) Patent Application Publication
(10) Pub. No.: US 2011/0111031 A1
(43) Pub. Date: May 12, 2011

(54) Drug Delivery Platforms Comprising Silk Fibroin Hydrogels and Uses Thereof

(76) Inventors: GUANG-LIANG JIANG, IRVINE, CA (US); ADAM L. COLLETTE, WESTMINSTER, MA (US); REBECCA L. HORAN, ARLINGTON, MA (US); JINGSONG CHEN, VIRGINIA BEACH, VA (US); GREGORY H. ALTMAN, ARLINGTON, MA (US); WHA BIN IM, IRVINE, CA (US)

(21) Appl. No.: 12/910,575

(22) Filed: Oct. 22, 2010

Related U.S. Application Data
(63) Continuation-in-part of application No. 12/873,563, filed on Sep. 1, 2010.
Application No. 12/873,563 is a continuation-in-part of application No. 12/764,039, filed on Apr. 20, 2010.
(60) Provisional application No. 61/170,895, filed on Apr. 20, 2009.

(51) Int. Cl. A61K 9/14; A61K 31/381; A61K 31/4025; A61K 31/4015; A61K 31/45; A61P 17/02; A61K 31/215; A61K 31/19; A61K 31/192; A61K 31/41

(52) U.S. Cl. 424/484; 424/499; 514/443; 514/422; 514/424; 514/327; 514/530; 514/573; 514/570; 514/382

(57) ABSTRACT

The present specification provides drug delivery platforms useful for the controlled release of a compound over time in an individual.

### CROSS REFERENCE

This patent application is a continuation-in-part that claims priority under 35 U.S.C. §120 to U.S. Non-Provisional patent application Ser. No. 12/873,563, filed Sep. 1, 2010, a continuation-in-part that claims priority under 35 U.S.C. §120 to U.S. Non-Provisional patent application Ser. No. 12/764,039, filed Apr. 20, 2010, a patent application that claims priority pursuant to 35 U.S.C. §119(e) to U.S. Provisional Patent Application Ser. No. 61/170,895 filed Apr. 20, 2009, each of which is hereby incorporated by reference in its entirety.

### FIELD OF THE INVENTION

The present specification discloses purified silk fibroin and method for purifying silk fibroins, biomaterials comprising silk fibroin with or without an amphiphilic peptide and methods for making biomaterials comprising silk fibroin and the use of silk fibroin biomaterials in a variety of medical uses, including, without limitation fillers for tissue space, templates for tissue reconstruction or regeneration, scaffolds for cells in tissue engineering applications and for disease models, a surface coating to improve medical device function, or as a platform for drug delivery.

### BACKGROUND

Silk refers to a filamentous product secreted by an organism such as a spider or silkworm. Fibroin is the primary structural component of silk. It is composed of monomeric units comprising an about 350 kDa heavy chain and an about 25 kDa light chain, and interspersed within the fibroin monomers is another about 25 kDa protein derived from the P25 gene. The ratio of heavy chain:light chain:P25 protein is about 6:6:1. Fibroin is secreted by the silk glands of the organism as a pair of complementary fibrils called “brins”. As fibroin brins leave the glands, they are coated with sericin, a glue-like substance which binds the brins together. Sericin is often antigenic and may be associated with an adverse tissue reaction when sericin-containing silk is implanted in vivo.

Silkworm silk fibers traditionally available in the commercial market are often termed “degummed”, which refers to the loosening and removal of a portion of the sericin coat surrounding the two fibroin brins through washing or extraction in hot soapy water. This degummed silk often contains or is recoated with sericin and other impurities in order to bind the plied multifilament together into a single fiber. Therefore, degummed silk, unless explicitly stated to the contrary, typically contains twenty percent to twenty-eight percent (by weight) sericin and can not be assumed to be sericin-free.

Silk fibers have historically been valued in surgery for their mechanical properties, particularly in the form of braided filaments used as a suture material. Residual sericin that may be contained in these materials stands as a potential obstacle to its use as a biomaterial as it does present the possibility for a heightened immune response. This sericin contamination may be substantially removed though, resulting in a virtually sericin-free fibroin which may be used either as fibers or dissolved and reconstituted in a number of forms. For example, natural silk from the silkworm Bombyx mori may be subjected to sericin extraction, spun into yarns then used to create a matrix with high tensile strength suitable for applications such as bioengineered ligaments and tendons. Use of regenerated silk materials has also been proposed for a number of medical purposes including wound protection, cell culture substrate, enzyme immobilization, soft contact lenses, and drug-release agents.

Silk fibroin devices whether native, dissolved, or reconstituted, do not typically contain cell-binding domains such as those found in collagen, fibronectin, and many other extracellular matrix (ECM) molecules. Fibroin is also strongly hydrophobic due to the 8-sheet-rich crystalline network of the core fibroin protein. These two factors couple to severely limit the capacity of native host cells to bind to and interact with implanted silk devices, as neither inflammatory cells like macrophages or reparative cells like fibroblasts are able to attach strongly, infiltrate and bioresorb the silk fibroin devices. In the case of virgin silk and black braided (wax or silicone coated) silk sutures, this is typically manifested in a harsh foreign-body response featuring peripheral encapsulation. Substantially sericin-free silk experiences a similar, though substantially less vigorous response when implanted. In essence, the host cells identify silk as a foreign body and opt to wall it off rather than interact with it. This severely limits the subsequent long-term potential of the device particularly relating to tissue in-growth and remodeling and potentially, the overall utility of the device. If it is possible to provide a more effective biomaterial formulation for mediating host-device interactions whereby cells are provided with a recognizable, acceptable and hence biocompatible surface, the biological, medicinal and surgical utility of silk is dramatically improved.

One possible means of introducing this improved cell-material interaction is to alter the silk fibroin biomaterial format into a more biocompatible matrix. Manipulating the silk fibroin to make it into a silk biomaterial is one particularly intriguing option because it consists of a silk protein network which is fully saturated with water, coupling the molecular resiliency of silk with the biocompatibility of a “wet” material. Generation of a silk biomaterial may be accomplished in short by breaking apart native silk fibroin polymers into its individual monomeric components using a solvent species, replacing the solvent with water, then inducing a combination of inter- and intra-molecular aggregation. It has been shown that the sol-gel transition can be selectively initiated by changing the concentration of the protein, temperature, pH and additive (e.g., ions and hygroscopic polymers such as poly(ethylene oxide) (PEO), poloxamer, and glycerol). Increasing the silk concentration and temperature may alter the time taken for silk gelation by increasing the frequency of molecular interactions, increasing the chances of polymer nucleation. Another means of accelerating silk gelation is through use of calcium ions which may interact with the hydrophilic blocks at the ends of silk molecules in solution prior to gelation. Decreasing pH and the addition of a hydrophilic polymer have been shown to enhance gelation, possibly by decreasing repulsion between individual silk molecules in solution and subsequently competing with silk fibroin molecules in solution for bound water, causing fibroin precipitation and aggregation.

Other silk fibroin gels have been produced by, for example, mixing an aqueous silk fibroin solution with protein derived biomaterials such as gelatin or chitosan. Recombinant proteins materials based on silk fibroin's structure have also been used to create self-assembling hydrogel structures. Another silk gel, a silk fibroin-poly-(vinyl alcohol) gel was created by freeze- or air-drying an aqueous solution, then reconstituting in water and allowing to self-assemble. Silk hydrogels have also been generated by either exposing the silk solution to temperature condition of 4° C. (Thermgel) or by adding thirty percent (v/v) glycerol (Glygel). Silk hydrogels created via a freeze-thaw process have not only been generated but also used in vitro as a cell culture scaffold.

The use of silk hydrogels as biomaterial matrices has also been explored in a number of ways. General research on hydrogels as platforms for drug delivery, specifically the release behavior of benfotiamine (a synthetic variant of vitamin B₁) coupled to silk hydrogel was investigated. The study revealed both silk concentration and addition of other compounds may factor in to the eventual release profile of the material. Similarly, the release of FITC-labeled dextran from a silk hydrogel could be manipulated by altering the silk concentrations within the gel.

Further studies of silk hydrogels have been performed in vivo as well. For example, the material has been used in vivo to provide scaffolding for repair of broken bones in rabbits and showed an accelerated healing rate relative to control animals. Of particular interest, the in situ study also illustrated that the particular formulation of silk hydrogel did not elicit an extensive immune response from the host.

Despite early promise with silk hydrogel formulations in vivo, sericin contamination remains a concern in their generation and use just as with native fibroin for reasons of biocompatibility as well as the potential for sericin to alter gelation kinetics. The existence of sericin molecules in the silk solution intermediate prior to gelation may also compromise final gel structural quality, i.e., the distribution of β-sheet structure. For these reasons the removal of sericin from silk fibroin biomaterial prior to hydrogel manufacture remains a concern. The potential for disruption of gelation kinetics and structure by contaminants also presents the need for development of a process which consistently ensures structural uniformity and biocompatibility.

### SUMMARY

The present specification provides novel drug delivery platforms that enable controlled release of a compound over time.

Thus, aspects of the present specification disclose a drug delivery platform comprising a biomaterial, the biomaterial comprising a substantially sericin-depleted silk fibroin and a compound having the structure of formula I,

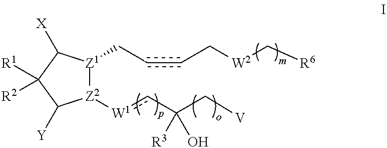

wherein each dashed line represents the presence or absence of a bond; R¹, R² and R³ are each independently selected from H or C₁-C₆ alkyl; R⁶ is CO₂H, CO₂R⁷, CON(R⁷)₂, CONHCH₂CH₂OH, CON(CH₂CH₂OH)₂, CH₂OR⁷, P(O)(OR⁷)₂, or

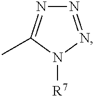

a pharmaceutically acceptable salt thereof, or a pharmaceutically acceptable amine salt thereof; R⁷ is H, C₁-C₆ alkyl or C₂-C₆ alkenyl; X and Y are each independently selected from H, OH, ═O, Cl, Br, I, or CF₃; Z¹ and Z² are each independently selected from CH or N; W¹ and W² are each independently selected from CH, CH₂, aryl or substituted aryl, heteroaryl, substituted heteroaryl; m is 0 to 6; o is 0 to 4; p is 0 or 1; and V is C₁-C₆ alkyl, aryl, substituted aryl, heteroaryl, or substituted heteroaryl. The biomaterial can be processed into a hydrogel, a sheet, a film, a porous material like a sponge or a foam, a solid, a fiber, a thread, a filament, or a mesh.

Other aspects of the present specification disclose a drug delivery platform comprising a substantially sericin-depleted silk fibroin and a compound having the structure of formula I as disclosed herein. The biomaterial can be processed into a hydrogel, a sheet, a film, a porous material like a sponge or a foam, a solid, a fiber, a thread, a filament, or a mesh.

Other aspects of the present specification disclose a drug delivery platform comprising a compound having the structure of formula I as disclosed herein encapsulated in crosslinked substantially sericin-depleted silk fibroin microspheres, wherein the microspheres include lipid components. In aspects, lipid composition is 1,2-dioleoyl-sn-glycero-3-phosphocholine (DOPC); 1,2-dioleoyl-sn-glycero-3-phophoethanolamine (DOPE); 1,2-dilauroyl-sn-glycero-3-phosphocholine (DLPC); or 1,2-dimyristoyl-sn-glycero-3-phosphocholine (DMPC). In other aspects, at least 75% of the microspheres are spherical or substantially spherical, and at least 75% of the microspheres have a diameter ranging from about 1.0 μm to about 3.0 μm.

Yet other aspects of the present specification disclose a process of encapsulating a biomaterial in substantially sericin-depleted silk fibroin microcapsules. The method comprises a) mixing a solution comprising substantially sericin-depleted silk fibroin with a compound having the structure of formula I as disclosed herein and a lipid composition; b) lyophilizing the mixture; c) combining the lyophilized mixture with a dehydration medium for a sufficient period of time to at least partially dehydrate the silk fibroin solution and induce β-sheet structures in the silk fibroin; and d) removing at least a portion of the lipids to produce a compound having the structure of formula I as disclosed herein encapsulated in a silk fibroin microcapsule. The lipid composition is as disclosed herein. In aspects, the dehydration medium is an alcohol, such as, e.g., methanol, ethanol, and propanol; ketones, such as, e.g., acetone; trihalomethanes, such as, e.g., chloroform; hygroscopic solutions, such as, e.g., polyethylene glycol solutions; or salt solutions.

Yet other aspects of the present specification disclose a process of making silk fibroin microspheres, the process comprising the steps of a) mixing a solution comprising a substantially sericin-depleted silk fibroin with a compound having the structure of formula I as disclosed herein and a lipid composition; b) lyophilizing the mixture; c) combining the lyophilized mixture with a dehydration medium for a sufficient period of time to at least partially dehydrate the silk fibroin solution and induce β-sheet structures in the silk fibroin; and d) removing at least a portion of the lipid composition to form silk fibroin microspheres comprising a compound having the structure of formula I as disclosed herein. The lipid composition and dehydration medium are as disclosed herein.

Yet other aspects of the present specification disclose a process of coating a substrate with one or a plurality of silk fibroin layers, process comprising the steps of a) mixing a solution comprising a substantially sericin-depleted silk fibroin with a compound having the structure of formula I as disclosed herein; b) contacting the substrate with the silk fibroin solution such that the solution forms a layer upon the substrate; and c) dehydrating the layer. Dehydration can be achieved by exposing the silk fibroin layer to a flow of air or other dehydrating gas. To form multiple layers of silk fibroin on a substrate, the substrate with the first dehydrated layer of silk fibroin is contacted a silk fibroin solution (with or without a compound having the structure of formula I as disclosed herein and/or other components as disclosed herein) such that the solution forms a second layer upon the dehydrated first layer, and dehydrating the second layer. These steps can be repeated until the desired number of dehydrated silk fibroin layers are deposited upon the substrate resulting in a silk fibroin layered coating on the substrate.

Yet other aspects of the present specification disclose a porous silk fibroin biomaterial comprising a three-dimensional silk fibroin body having interconnected pores, wherein the pores. In aspects, the pores have a diameter of 10 μm to 1000 μm. In other aspects, the porous material has a compressive modulus of at least 100 kPa.

Still other aspects of the present specification disclose a method of treating glaucoma in an individual, the method comprising the step of administering a drug delivery platform as disclosed herein into an eye of the individual, wherein administration reduces a symptom associated with glaucoma, thereby treating the glaucoma.

Still other aspects of the present specification disclose a method of treating elevated intraocular pressure in an individual, the method comprising the step of administering a drug delivery platform as disclosed herein into an eye of the individual, wherein administration reduces intraocular pressure, thereby treating the elevated intraocular pressure.

Still other aspects of the present specification disclose a method of treating corneal haze or opacity in an individual, the method comprising the step of administering a drug delivery platform as disclosed herein into an eye of the individual, wherein administration reduces a symptom associated with corneal haze or opacity, thereby treating the corneal haze or opacity.

Still other aspects of the present specification disclose a method of treating inflammatory bowel disease in an individual, the method comprising the step of administering a drug delivery platform as disclosed herein to the individual, wherein administration reduces a symptom associated with inflammatory bowel disease, thereby treating the inflammatory bowel disease.

Still other aspects of the present specification disclose a method of treating a wound in an individual, the method comprising the step of administering a drug delivery platform of as disclosed herein to the wound of the individual, wherein administration promotes healing of the wound, thereby treating the wound.

### DESCRIPTION OF THE DRAWINGS

FIG. 1 illustrates the impact of 23RGD on the gelation times of silk hydrogels manufactured under various circumstances for example without enhancers or with a water/23RGD enhancer (FIG. 1A), or with an ethanol enhancer or combined ethanol-23RGD enhancers (FIG. 1B).

FIG. 2 is a graph of HPLC data illustrating the integration of 23RGD and stability of its binding to 4% silk gel material. Data are shown for both total peak area and calculated 23RGD:silk molar ratio based on a 23RGD standard curve.

FIG. 3 is a graph comparing gel dry mass component at different RGD concentrations for 2% silk gels (A), 4% silk gels (B), and 6% gels (C). * Samples differ significantly, p<0.05; † sample differs significantly from all others; ‡ all samples differ significantly.

FIG. 4 illustrates the impact upon silk hydrogel water absorption and retention as identified in a gel drying assay.

FIG. 5 shows a comparison of the percent mass loss over time due to bioresorption of samples cast by PG and EEG methods (A), cast from increasing silk concentrations (B), and cast using increasing RGD concentrations (C). * Samples differ significantly, p<0.05; † sample differs significantly from all others; ‡ all samples differ significantly.

FIG. 6 illustrates wet mass loss due to proteolytic bioresorption of silk hydrogels enhanced by a combination of 23RGD and ethanol at increasing concentrations of 23RGD.

FIG. 7 is an illustration of the bioresorption behavior of 23RGD-enhanced and non-23RGD-enhanced silk hydrogels when incubated in a protease solution.

FIG. 8 shows structural features observed by Fourier-Transform Infrared (FTIR) spectroscopy of 4% silk fibroin hydrogel devices which are enhanced by ethanol alone, and two 23RGD-ethanol enhancers: the full spectra (FIG. 8A); the Amide I Band (1700-1600 cm⁻¹) (FIG. 8B); β-sheet structure (1700 cm⁻¹) (FIG. 8C); α-helix and random coil conformations (1654 cm⁻¹ and 1645 cm⁻¹ respectively) (FIG. 8D); and β-sheet structure (1622 cm⁻¹) (FIG. 8E).

FIG. 9 shows a comparative FTIR spectra illustrating the effects of differing gelation techniques on gel protein structure before (Day 0) and after (Day 4) proteolytic bioresorption. Groups assessed included samples cast by PG and EEG methods (A), cast from increasing silk concentrations (B), and cast using increasing RGD concentrations (C).

FIG. 10 shows representative micrographs of H&E-stained histological sections collected from silk gels implanted subcutaneously in rats. Samples of 4% silk fibroin hydrogel formed by passive gelation (4P), 4% silk fibroin hydrogel formed by ethanol-enhanced gelation (4E), and 6% silk fibroin hydrogel formed by ethanol-enhanced gelation (6E) were compared at 7 days (A, B, and E respectively) with 4E and 6E samples compared again at days 28 (C and F) and 57 (D and G).

FIG. 11 shows representative gross photographs of 8% silk fibroin hydrogel devices both unmodified (A) and 23RGD-enhanced (D) after a two-week subcutaneous incubation in Lewis rats. Also shown are micrographs resultant from H & E stains of the unmodified (B and C) and 23RGD-coupled (E and F) samples at 10× and 20× magnification.

FIG. 12 shows representative histology collected from a thirteen-week study of 4% 3:1 23RGD-enhanced silk hydrogel blended with 25% saline (left panels, H&E stain Trichrome stain) and ZYPLAST™ (right panels H&E stain, Trichrome stain) and injected into the intradermis of guinea pig. Each material type exhibited some clear evidence of implanted device in 75% of their respective implant sites.

FIG. 13 shows representative micrographs of H&E-stained histological sections collected from Day 28 explants of 4% silk fibroin, 10% saline (A); 4% silk fibroin, 1:1 23RGD, 10% saline (B); 6% silk fibroin, 1:1 23RGD, 10% saline (C); ZYPLAST™ (D); 4% silk fibroin, 25% saline (E); 4% silk fibroin, 1:1 23RGD, 25% saline (F); 6% silk fibroin, 10% saline (G); HYLAFORM™ (H); 6% silk fibroin, 25% saline (I); 4% silk fibroin, 3:1 23RGD, 25% saline (J); and 6% silk fibroin, 1:1 23RGD, 25% saline (K).

FIG. 14 shows representative micrographs of Day 92 histological sections of 4% silk fibroin, 3:1 23RGD, 25% saline (A-D) and ZYPLAST™ samples (E-H) stained with H&E at 4× (A and E), 10× (B and F), stained with Masson's Trichrome at 10× (C and G) and under polarized light at 10× (D and H).

FIG. 15 is a photograph of a custom-built testing jig used in conjunction with an Instron 8511 (Instron Corporation, Canton Mass.) in conjunction with Series IX software and a 100 N load cell for characterizing the injection forces associated with forcing silk gel through a 30 g needle.

FIG. 16 illustrates the average extrusion force data from mechanical testing of various silk gel formulations illustrating the effects of changing comminution method (A), saline concentration (B), silk concentration (C), and RGD content (D). * Samples differ significantly, p<0.05; † sample differs significantly from all others in group at same strain rate; ‡ all samples in group differ significantly from all others in group at same strain rate.

FIG. 17 shows representative ESEM micrographs of selected RGD/ethanol-induced silk precipitates generated from the previously mentioned formulations. BASE (A), SCVLO (B), RHI (C), RLO (D), AVHI (E), ECLO (F), AVLO (G), and 3R 6.7:1 (H) are shown at 200× magnification.

FIG. 18 shows a comparison of the total dry mass of precipitate recovered from each silk precipitate formulation (n=4 for each type) after being subjected to a 96-hour lyophilization process. Data are grouped to compare the effects of changing volume ratio of accelerant added (A), concentration of 23RGD in the accelerant (B), changing the initial silk concentration (C), and changing the concentration of ethanol in the accelerant (D). *—Significant difference, p<0.05; †—Group differs significantly from all others.

FIG. 19 shows a comparison of the percentage of dry mass in each of precipitate recovered (n=4 for each type) after being subjected to a 96-hour lyophilization process. Data are grouped to compare the effects of changing volume ratio of accelerant added (A), concentration of 23RGD in the accelerant (B), changing the initial silk concentration (C), and changing the concentration of ethanol in the accelerant (D). *—Significant difference, p<0.05; †—Group differs significantly from all others.

FIG. 20 shows representative FTIR spectra of the Amide I band for 23RGD/ethanol-induced silk precipitates immediately after processing (D0). Spectra are grouped to compare the effects of changing volume ratio of accelerant added (A), concentration of 23RGD in the accelerant (B), changing the initial silk concentration (C), and changing the concentration of ethanol in the accelerant (D).

FIG. 21 is a representative micrograph of Congo red-stained 23RGD/ethanol-induced silk precipitates under polarized light at 20× magnification. A lack of emerald-green birefringence indicates a negative result in testing for amyloid fibril formation.

FIG. 22 shows comparison of 23RGD:silk molar ratio in each of precipitate recovered. Data are grouped to compare the effects of changing volume ratio of accelerant added (A), concentration of 23RGD in the accelerant (B), changing the initial silk concentration (C), and changing the concentration of ethanol in the accelerant (D). *—Significant difference, p<0.05; †—Group differs significantly from all others; ‡—All groups differ significantly.

FIG. 23 shows a representative FTIR spectra of the Amide I band are shown for 23RGD/ethanol-induced silk precipitates initially (D0) and after proteolytic bioresorption (D2). Spectra are grouped to compare the effects of changing volume ratio of accelerant added (A), concentration of 23RGD in the accelerant (B), changing the initial silk concentration (C), and changing the concentration of ethanol in the accelerant (D).

### DETAILED DESCRIPTION

Aspects of the present specification disclose, in part, a drug delivery platform. The silk fibroin biomaterials disclosed herein can be used as a platform for drug delivery in order to achieve a controlled compound release profile over time. Furthermore, the silk fibroin biomaterials can serve as the outer coating for coaxial drug delivery platforms.

For example, the silk biomaterials disclosed herein can be formed with a pharmaceutical agent, like the compounds disclosed herein, either entrained in or bound to the biomaterial and injected, implanted, or delivered orally into the body. For extended or sustained-drug delivery, silk biomaterials can manipulated to be highly resistant to bioresorption and hydrophobic under certain conditions (e.g., very high β-sheet content) which discourages cell/tissue ingrowth. This in turn leads to prolonged gel bioresorption and, by extension, prolonged drug release such as, e.g., sustained drug release or extended drug release. The silk fibroin biomaterials can be produced as a gel or as a solid. The pharmaceutically-active drug can be, without limitation, proteins, peptides, steroids, antibiotics, vitamins, simple sugars, genes, transfected or non-transfected cells. To control the drug release profile, the pharmaceutically-active drug can be first mixed with silk solutions then form a biomaterial. This silk biomaterials can be ground into smaller particles mixed with an additional phase acting as a carrier either with or without a viscosity inducing component, a surfactant, and/or an included lubricant fluid like saline. The therapeutic-bound silk biomaterials can also be further crosslinked to enhance the stability to extend the release period.

A drug delivery platform includes both a sustained release and an extended release drug delivery platforms. As used herein, the term “extended release” refers to the release of a compound disclosed herein over a period of time of less than about 7 days. As used herein, the term “sustained release” refers to the release of a compound disclosed herein over a period of about 7 days or more.

In aspects of this embodiment, an extended drug delivery platform releases a compound disclosed herein with substantially first order release kinetics over a period of, e.g., about 1 day, about 2 days, about 3 days, about 4 days, about 5 days, or about 6 days after administration. In other aspects of this embodiment, a drug delivery platform releases a compound disclosed herein with substantially first order release kinetics over a period of, e.g., at most 1 day, at most 2 days, at most 3 days, at most 4 days, at most 5 days, or at most 6 days after administration.

In aspects of this embodiment, a sustained release drug delivery platform releases a compound disclosed herein with substantially first order release kinetics over a period of, e.g., about 7 days, about 15 days, about 30 days, about 45 days, about 60 days, about 75 days, or about 90 days after administration. In other aspects of this embodiment, a sustained release drug delivery platform releases a compound disclosed herein with substantially first order release kinetics over a period of, e.g., at least 7 days after administration, at least 15 days, at least 30 days, at least 45 days, at least 60 days, at least 75 days, or at least 90 days after administration.

Aspects of the present specification provide, in part, a depolymerized silk fibroin. As used herein, the term “depolymerized silk fibroin” is synonymous with “dissolved silk” and “dissolved silk fibroin” and refers to silk fibroin existing primarily as monomers or other lower oligomeric units. Treatment of naturally-occurring fibrous silk with a dissolution agent, such as, e.g., a chaotropic agent results in depolymerized silk fibroin. The depolymerized silk fibroin is an intermediate in the silk biomaterial production process and a direct precursor to the biomaterial. The depolymerized silk fibroin can be made from raw cocoons, previously degummed silk or any other partially cleaned silk. This may also include material commonly termed as “waste” from the reeling process. A particular source of raw silk is from common domesticated silkworm B. mori, though several other sources of silk may be appropriate. This includes other species of Bombycidae including Antheraea pernyi, Antheraea yamamai, Antheraea mylitta, Antheraea assama, and Philosamia cynthia ricini, as well as silk producing members of the families Saturnidae, Thaumetopoeidae, and the order Araneae. The material may also be obtained from other spider, caterpillar, or recombinant sources.

Aspects of the present specification provide, in part, a biomaterial comprising a silk fibroin. As used herein, the term “silk fibroin” is synonymous with “polymerized silk fibroin” and refers to silk fibroin existing primarily as a polymer.

The biomaterials disclosed herein provide for a depolymerized silk fibroin and/or silk fibroin that is substantially free of sericin. Methods for performing sericin extraction have been described in Altman, et al., Matrix for the Production of Tissue Engineered Ligaments, Tendons and Other Tissue, U.S. Pat. No. 6,902,932, which is hereby incorporated by reference in its entirety. That application refers to cleaned fibroin fibers spun into yarns, used to create a porous, elastic matrix suitable as a substrate for applications requiring very high tensile strength, such as bioengineered ligaments and tendons.

Extractants such as urea solution, hot water, enzyme solutions including papain among others which are known in the art to remove sericin from fibroin would also be acceptable for generation of the silk. Mechanical methods may also be used for the removal of sericin from silk fibroin. This includes but is not limited to ultrasound, abrasive scrubbing and fluid flow. The rinse post-extraction is conducted preferably with vigorous agitation to remove substantially any ionic contaminants, soluble, and insoluble debris present on the silk as monitored through microscopy and solution electrochemical measurements. A criterion is that the extractant predictably and repeatably remove the sericin coat of the source silk without significantly compromising the molecular structure of the fibroin. For example, an extraction may be evaluated for sericin removal via mass loss, amino acid content analysis, and scanning electron microscopy. Fibroin degradation may in turn be monitored by FTIR analysis, standard protein gel electrophoresis and SEM.

In certain cases, the silk utilized for generation of a silk biomaterial has been substantially depleted of its native sericin content (i.e., ≦4% (w/w) residual sericin in the final extracted silk). Alternatively, higher concentrations of residual sericin may be present following extraction or the extraction step may be omitted. In aspects of this embodiment, the sericin-depleted silk fibroin has, e.g., about 1% (w/w), about 2% (w/w), about 3% (w/w), or about 4% (w/w) residual sericin. In other aspects of this embodiment, the sericin-depleted silk fibroin has, e.g., at most 1% (w/w), at most 2% (w/w), at most 3% (w/w), or at most 4% (w/w) residual sericin. In yet other aspects of this embodiment, the sericin-depleted silk fibroin has, e.g., about 1% (w/w) to about 2% (w/w) residual sericin, about 1% (w/w) to about 3% (w/w) residual sericin, or about 1% (w/w) to about 4% (w/w) residual sericin.

In certain cases, the silk used to make a silk biomaterial is entirely free of its native sericin content. As used herein, the term “entirely free (i.e. “consisting of” terminology) means that within the detection range of the instrument or process being used, the substance cannot be detected or its presence cannot be confirmed. In other cases, the silk utilized for generation of a silk biomaterial is essentially free of its native sericin content. As used herein, the term “essentially free” (or “consisting essentially of”) means that only trace amounts of the substance can be detected. Additionally, the possibility exists for deliberately modifying biomaterial properties through controlled partial removal of silk sericin or deliberate enrichment of source silk with sericin. This may function to improve biomaterial hydrophilicity and eventual host acceptance in particular biological settings despite sericin antigenicity.

After initial degumming or sericin removal from fibrous silk, the silk is rinsed free of gross particulate debris. It is of concern to remove such particles as either solvent (i.e., specific solvent of interest for device generation) soluble or insoluble compounds may profoundly affect the outcome of the biomaterial generated from the intermediate solution. For example, insoluble compounds may serve as nucleation points, accelerating the gelation phenomenon and potentially altering subsequent biomaterial structure. Soluble compounds may also serve to interface with the protein network of the biomaterial, altering the organizational state of the device. Either type of compound could also compromise biocompatibility of the device.

Prior to dissolution, the prepared silk may be subjected to association of various molecules. The binding between these compounds and the silk fibroin may be unaffected by the dissolving agent used for preparation of silk solution intermediate. The method for coupling the modifying compound to the prepared silk may vary dependent upon the specific nature of the bond desired between silk sequence and the modifier. Methods are not limited to but may include hydrogen bonding through affinity adsorption, covalent crosslinking of compounds or sequential binding of inactive and active compounds. These molecules may include, but would not be limited to, inorganic compounds, peptides, proteins, glycoproteins, proteoglycans, ionic compounds, natural, and synthetic polymers. Such peptides, proteins, glycoproteins and proteoglycans may include classes of molecules generally referred to as “growth factors”, “cytokines”, “chemokines”, and “extracellular matrix compounds”. These compounds might include such things as surface receptor binding motifs like arginine-glycine-aspartic acid (RGD), growth factors like basic fibroblast growth factor (bFGF), platelet derived growth factor (PDGF), transforming growth factor (TGF), cytokines like tumor necrosis factor (TNF), interferon (IFN), interleukins (IL), and structural sequences including collagen, elastin, hyaluronic acid and others. Additionally recombinant, synthetic, or non-native polymeric compounds might be used as decoration including chitin, poly-lactic acid (PLA), and poly-glycolic acid (PGA). Other compounds linked to the material may include classes of molecules generally referred to as tracers, contrasting agents, aptamers, avimers, peptide nucleic acids and modified polysaccharide coatings.

For example, the initially dissolved silk may be generated by a 4 hour digestion at 60° C. of pure silk fibroin at a concentration of 200 g/L in a 9.3 M aqueous solution of lithium bromide to a silk concentration of 20% (w/v). This process may be conducted by other means provided that they deliver a similar degree of dissociation to that provided by a 4 hour digestion at 60° C. of pure silk fibroin at a concentration of 200 g/L in a 9.3 M aqueous solution of lithium bromide. The primary goal of this is to create uniformly and repeatably dissociated silk fibroin molecules to ensure similar fibroin solution properties and, subsequently, device properties. Less substantially dissociated silk solution may have altered gelation kinetics resulting in differing final gel properties. The degree of dissociation may be indicated by Fourier-transform Infrared Spectroscopy (FTIR) or x-ray diffraction (XRD) and other modalities that quantitatively and qualitatively measure protein structure. Additionally, one may confirm that heavy and light chain domains of the silk fibroin dimer have remained intact following silk processing and dissolution. This may be achieved by methods such as standard protein sodium-dodecyl-sulfate polyacrylamide gel electrophoresis (SDS-PAGE) which assess molecular weight of the independent silk fibroin domains.

System parameters which may be modified in the initial dissolution of silk include but are not limited to solvent type, silk concentration, temperature, pressure, and addition of mechanical disruptive forces. Solvent types other than aqueous lithium bromide may include but are not limited to aqueous solutions, alcohol solutions, 1,1,1,3,3,3-hexafluoro-2-propanol, and hexafluoroacetone, 1-butyl-3-methylimidazolium. These solvents may be further enhanced by addition of urea or ionic species including lithium bromide, calcium chloride, lithium thiocyanate, zinc chloride, magnesium salts, sodium thiocyanate, and other lithium and calcium halides would be useful for such an application. These solvents may also be modified through adjustment of pH either by addition of acidic of basic compounds.

Further tailoring of the solvent system may be achieved through modification of the temperature and pressure of the solution, as ideal dissolution conditions will vary by solvent selected and enhancers added. Mechanical mixing methods employed may also vary by solvent type and may vary from general agitation and mixing to ultrasonic disruption of the protein aggregates. Additionally, the resultant dissolved silk concentration may be tailored to range from about 1% (w/v) to about 30% (w/v). It may be possible to expand this range to include higher fractions of dissolved silk depending upon the specific solvent system utilized. In one example, following initial dissolution of the processed silk, the silk protein may be left in a pure aqueous solution at 8% (w/v) silk. This is accomplished by removal of the residual solvent system while simultaneously ensuring that the aqueous component of the silk solution is never fully removed nor compromised. In a situation which involves an initial solution of 200 g/L silk in a 9.3 M aqueous solution of lithium bromide, this end is accomplished by a dialysis step.

In aspects of this embodiment, the depolymerized silk fibroin (dissolved silk fibroin) has a concentration of, e.g., about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 12% (w/v), about 15% (w/v), about 18% (w/v), about 20% (w/v), about 25% (w/v), or about 30% (w/v). In other aspects of this embodiment, the depolymerized silk fibroin (dissolved silk fibroin) has a concentration of, e.g., at least 1% (w/v), at least 2% (w/v), at least 3% (w/v), at least 4% (w/v), at least 5% (w/v), at least 6% (w/v), at least 7% (w/v), at least 8% (w/v), at least 9% (w/v), at least 10% (w/v), at least 12% (w/v), at least 15% (w/v), at least 18% (w/v), at least 20% (w/v), at least 25% (w/v), or at least 30% (w/v). In yet other aspects of this embodiment, the depolymerized silk fibroin (dissolved silk fibroin) has a concentration of, e.g., about 1% (w/v) to about 5% (w/v), about 1% (w/v) to about 10% (w/v), about 1% (w/v) to about 15% (w/v), about 1% (w/v) to about 20% (w/v), about 1% (w/v) to about 25% (w/v), about 1% (w/v) to about 30% (w/v), about 5% (w/v) to about 10% (w/v), about 5% (w/v) to about 15% (w/v), about 5% (w/v) to about 20% (w/v), about 5% (w/v) to about 25% (w/v), about 5% (w/v) to about 30% (w/v), about 10% (w/v) to about 15% (w/v), about 10% (w/v) to about 20% (w/v), about 10% (w/v) to about 25% (w/v), or about 10% (w/v) to about 30% (w/v).

A silk solution can be concentrated by dialysis. The parameters applied to the dialysis step may be altered according to the specific needs or requirements of the particular solution system involved. Such parameters include, e.g., membrane composition or pore size, buffer phase, water purity or amount and type of hygroscopic polymers. For example, if necessary, the silk solution can be concentrated by dialysis against a hygroscopic polymer, for example, PEG, a polyethylene oxide or amylase. The apparatus used for dialysis can be cassettes, tubing, or any other semi-permeable membrane.

Example dialysis conditions include a 3 mL-12 mL sample volume dialysis cassettes with 3.5 kD molecular weight cutoff cellulose membranes dialyzed for three days against ultra-pure water with a series of six changes at regular intervals while stirring constantly. Each cassette, 3 mL-12 mL cartridge size, may be loaded (for example via 20-mL syringe) with 12 mL of a 20% solution of silk dissolved in 9.3 M lithium bromide via an 18 gauge needle. The resultant silk solution may be 8%±0.5% (w/v). The silk solution may be stored at a range of −80° C. to 37° C., such as 4° C. prior to use. One method is to dialyze the solution against water using a 3.5 kD molecular weight cutoff cellulose membrane, for example, at one 12 mL cartridge per 1 L water in a 4 L beaker with stirring for 48 hours or 72 hours. Water may be changed several times during the dialysis, for example at 1 hour, 4 hours, 12 hours, 24 hours, and 36 hours (total of six rinses). In other embodiments, this membrane may take the shape of a cassette, tubing or any other semi-permeable membrane in a batch, semi-continuous or continuous system. If desired, the concentration of silk in solution may be raised following the original dialysis step by inclusion of a second dialysis against a hygroscopic polymer such as PEG, a poly(ethylene oxide) or amylase.

Insoluble debris may be removed from the dialyzed silk solution by centrifugation or filtration. For example, the dialyzed silk may be removed from the cassette and centrifuged at 30,000 g RCF for 30 minutes at 4° C. The resulting supernatant may be collected and centrifuged again under identical conditions, and the remaining supernatant collected and stored at 4° C. The silk solution may also be evaluated via X-ray photoelectron spectroscopy to check for lithium bromide residue and dry massed to check solution for dry protein mass, concentration, etc.

Additionally, dependent upon the initial silk solvent, it might be desirable to remove portions of either the silk phase or solvent phase from the solution via an affinity column separation. This could be useful in either selectively binding specific solvent molecules or specific solute molecules to be eluted later in a new solvent. The possibility also exists for a lyophilization of the depolymerized silk fibroin followed by a reconstitution step. This would be useful when removing a solvent is unlikely to leave residue behind. In the case of a lyophilized solution, the type of solvent used for reconstitution can be tailored for the process at hand. Desirable solvents might include but are not limited to aqueous alcohol solutions, aqueous solutions with altered pH, and various organic solutions. These solvents may be selected based upon a number of parameters which may include but are not limited to an enhanced gelation rate, altered gel crystalline structure, altered solution intermediate shelf-life, altered silk solubility, and ability to interact with environmental milieu such as temperature and humidity.

Aspects of the present specification provide, in part, a silk fibroin biomaterial comprising an amphiphilic peptide. As used herein, the term “amphiphilic peptide” refers to a peptide that includes both hydrophobic and hydrophilic properties. Amphiphilic peptide interact strongly with biological membranes by insertion of the hydrophobic part into the lipid membrane, while exposing the hydrophilic part to the aqueous environment. Particular embodiments of biomaterials include silk fibroin, silk fibroin with amphiphilic peptide, silk fibroin with alcohol and amphiphilic peptide, and silk fibroin with alcohol, amphiphilic peptide, and saline/PBS. The amount, relative ratio and sequence of adding the components will change according to the specific requirement for the device.

An example of an amphiphilic peptide is a 23RGD peptide having the amino acid sequence: HOOC-Gly-Arg-Gly-Asp-Ile-Pro-Ala-Ser-Ser-Lys-Gly-Gly-Gly-Gly-Ser-Arg-Leu-Leu-Leu-Leu-Leu-Leu-Arg-NH₂ (SEQ ID NO: 1). Optionally, each of the arginine residues may be of the D-form, which may stabilize the RG bond to serine proteases. Additionally, the COO-terminus may be acylated to block proteolysis. This example 23RGD has the amino acid sequence Ac-GdRGDIPASSKG₄SdRL_{6d}R—NH₂ (SEQ ID NO: 2). It may be advantageous to include a spacer domain in the RGD peptide, for example, a peptide such as SG₄KSSAP (SEQ ID NO: 3) may present the RGD on the surface of the silk biomaterial by optimally separating the cell attachment domain from the bonding sequence at the end of the peptide. The optional leucine tails of this example may interact in a fashion analogous to a leucine zipper, and be driven by entropy from an aqueous solution to form an approximation of a Langmuir-Blodgett (LB), monomolecular film on the surface of materials exposed to such solutions, thus presenting a ‘carpet’ of RGD attachment sites on those surfaces.

Other proteins or peptides may be used instead of RGD peptides if such proteins or peptides have the desired characteristics. Example characteristics include hydrophilic domains that can interfere/enhance/affect silk gelation, and/or cell integrin binding domains that enhance cell adhesion, spreading, and migration. Non-limiting examples of such non-RGD integrins include, KQAGDV (SEQ ID NO: 4), PHSRN (SEQ ID NO: 5), YIGSR (SEQ ID NO: 6), CDPGYIGSR (SEQ ID NO: 7), IKVAV (SEQ ID NO: 8), RNIAEIIKDI (SEQ ID NO: 9), YFQRYLI (SEQ ID NO: 10), PDSGR (SEQ ID NO: 11), FHRRIKA (SEQ ID NO: 12), PRRARV (SEQ ID NO: 13), and WQPPRAR1 (SEQ ID NO: 14). See also Hersel et al., 24 Biomaterials 4285-415 (2003).

In aspects of this embodiment, a biomaterial comprises a molar ratio of amphiphilic peptide to silk fibroin of, e.g., about 100:1, about 90:1, about 80:1, about 70:1, about 60:1, about 50:1, about 40:1, about 30:1, about 20:1, about 10:1, about 7:1, about 5:1, about 3:1, about 1:1, about 1:3, about 1:5, about 1:7, about 1:10, about 1:20, about 1:30, about 1:40, about 1:50, about 1:60, about 1:70, about 1:80, or about 1:90, or about 1:100. In other aspects of this embodiment, a biomaterial comprises a molar ratio of amphiphilic peptide to silk fibroin of, e.g., at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, or at least 1:90, or at least 1:100. In yet other aspects of this embodiment, a biomaterial comprises a molar ratio of amphiphilic peptide to silk fibroin of, e.g., at most 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, or at most 1:90, or at most 1:100. In still other aspects of this embodiment, a biomaterial comprises a molar ratio of amphiphilic peptide to silk fibroin of, e.g., about 100:1 to about 1:100; about 90:1 to about 1:90; about 80:1 to about 1:80; about 70:1 to about 1:70; about 60:1 to about 1:60; about 50:1 to about 1:50; about 40:1 to about 1:40; about 30:1 to about 1:30; about 20:1 to about 1:20; about 10:1 to about 1:10; about 7:1 to about 1:7; about 5:1 to about 1:5; or about 3:1 to about 1:3.

The use of an amphiphilic peptide not only alters the protein structure characteristics of silk fibroin protein, but in so doing alters its resistance to proteolytic bioresorption in vitro. These alterations in proteolytic bioresorption resistance stem from aspects of the protein structure alteration as α-helix and random coil are typically thought to be less stable and therefore more susceptible to proteolytic bioresorption than β-sheet regions of silk. β-turn and β-strand regions of the biomaterial disclosed herein are most resistant to proteolytic bioresorption as opposed to regions of α-helixes and random coils. Through deliberate manipulation of this protein structure by means of controlled solution concentration and addition of enhancer factors (type, concentration, and driving gradient), gelation kinetics and resultant gel properties can be controlled to deliver optimal outcomes in terms of degradative and resultant biological behaviors. The impact of amphiphilic peptide addition to a silk biomaterial is evident upon examination of implantation studies conducted in vivo, both subcutaneously in rats and intradermally in the dermis of guinea pigs. See Example 10.

Aspects of the present specification provide, in part, a silk fibroin biomaterial comprising a five-amino acid peptide “tail” capable of linking or conjugating a molecule X to a silk molecule or fibroin when the molecule X is attached to the tail. A molecule X is any entity, natural or synthetic, that can be useful in the context of disclosed silk fibroin biomaterials. As used herein, the term “linking” or “conjugating” in the context of molecule X refers to an indirect physical attachment of molecule X to silk fibroin via a third entity, e.g., the five-amino acid peptide “tail”. In one embodiment, the tail binds to silk fibroin by hydrophobic interaction to the silk fibroin. Alternatively, the “tail” binds the silk molecules by hydrogen bonding and/or covalent bonding. It is envisioned that the “tail” can bind silk fibroins by a combination of hydrophobic interactions, hydrogen bonds, and covalent bonds. By attaching a molecule X to a “tail” described herein, it is possible to indirectly link the molecule X to silk fibroin via the tail, and thus to the silk fibroin biomaterials described herein.

In one embodiment, the molecule X is attached to a tail at the carboxyl (COON) end of the five-amino acid peptide. In another embodiment, the molecule X is attached to a tail at the amino (NH₂) end of the five-amino acid peptide. In yet another embodiment, the five-amino acid peptide “tail” comprises hydrophobic and/or apolar (non polar) amino acid residues such as valine, leucine, isoleucine, phenylalanine, tryptophan, methionine, cysteine, alanine, tyrosine, serine, proline, histidine, threonine and glycine. Various combinations of hydrophobic and/or apolar amino acid residues are possible, for e.g. LLLLL (SEQ ID NO: 15), LLFFL (SEQ ID NO: 16), LFLWL (SEQ ID NO: 17), FLWLL (SEQ ID NO: 18) and LALGL (SEQ ID NO: 19). In other embodiments, the tail comprises any combination of the twenty standard conventional amino acid residues. In other embodiments, the tail comprises hydrophobic and/or apolar (non polar) and amino acids residues with hydrophobic side chains, e.g. arginine and lysine. As used herein, the term “comprising” or “comprises” means that other elements can also be present in addition to the defined elements presented. The use of “comprising” indicates inclusion rather than limitation.

In one embodiment, the five-amino acid peptide “tail” capable of linking or conjugating a molecule X to a silk molecule or fibroin when the molecule X is attached to the tail comprise more than five amino acid residues, e.g. six or seven hydrophobic and/or apolar amino acids, such as LLLLLL (SEQ ID NO: 20). In another embodiment, the five-amino acid peptide “tail” comprises amino acid residues that are part hydrophobic (i.e. the part of the side-chain nearest to the protein main-chain), for e.g. arginine and lysine. In one embodiment, the part hydrophobic amino acid residues flank the five-amino acid peptide “tail” such as in RLLLLLR (SEQ ID NO: 21), KLLLLLR (SEQ ID NO: 22) and KLLLLLK (SEQ ID NO: 23).

In one embodiment, the five-amino acid peptide “tail” is separated from a molecule X by a spacer peptide. Spacer peptides should generally have non-polar amino acid residues, such as, glycine and proline.

In one embodiment, the spacer comprises unnatural amino acid residues such as nor amino acids and keto-substituted amino acids. Such unnatural amino acid residues are well known to one skilled in the art. In one embodiment, the spacer peptide is attached to a tail at the carboxyl end of the five-amino acid peptide. In another embodiment, the spacer is attached to a tail at the amino end of the five-amino acid peptide.

The length of a space peptide is variable. The spacer serves to link the molecule X and tail together and also to provide steric freedom to the molecule X, allowing for proper orientation of a molecule X (e.g. cell binding domains such as the RGD domain) and the correct interaction of the molecule X with cells in vivo. A spacer which is too short can prevent the molecule X from being properly functional (i.e., holding it too tight to the silk molecules and away from cells), a spacer which is too long can cause undesired effects as well (i.e., non-specific association of peptides or shortened efficacy from peptide due to spacer breakage). In one embodiment, the number of amino acid residues in a spacer can range form 1 to 300. In one embodiment, the spacer comprises a single amino acid residue, such as a G or a P. Examples of spacers with more amino acid residues are GSPGISGGGGGILE (SEQ ID NO: 24) and SGGGGKSSAPI (SEQ ID NO: 25).

In one embodiment, the molecule X is any biological molecule or fragment thereof. Examples biological molecules include but are not limited to growth factors, hormones, cytokines, chemokines, extracellular matrix compounds, osteogenic protein (OP), bone morphogenetic protein (BMP), growth and differentiation factor (GDF), transforming growth factor (TGF), epidermal growth factor (EGF), vascular endothelial growth factor (VEGF), interleukin (IL), platelet derived growth factor (PDGF), fibroblast growth factor (FGF), insulin-like growth factor (IGF), basic fibroblast growth factor (BFGF), fibroblast activation protein (FAP), disintegrin, metalloproteinase (ADAM), matrix metalloproteinase (MMP), connective tissue growth factor (CTGF), stromal derived growth factor (SDGF), keratinocyte growth factor (KGF), tumor necrosis factor (TNF), interferon (IFN), erythropoietin (EPO), hepatocyte growth factor (HGF), thrombopoietin (TPO), granulocyte colony stimulating factor (GCSF), granulocyte macrophage colony stimulating factor (GMCSF), myostatin (GDF-8), collagen, resilin, elastin, laminin, hyaluronic acid, decorin, actin, and tubulin. Examples fragments of biological molecules include but are not limited to known cell integrin binding domains including but not limited to RGD, KQAGDV (SEQ ID NO: 4), PHSRN (SEQ ID NO: 5), YIGSR (SEQ ID NO: 6), CDPGYIGSR (SEQ ID NO: 7), IKVAV (SEQ ID NO: 8), RNIAEIIKDI (SEQ ID NO: 9), YFQRYLI (SEQ ID NO: 10), PDSGR (SEQ ID NO: 11), FHRRIKA (SEQ ID NO: 12), PRRARV (SEQ ID NO: 13), and WQPPRAR1 (SEQ ID NO: 14).

In other embodiments, the molecule X is any recombinant, synthetic, or non-native polymeric compounds. Examples include but are not limited to chitin, poly-lactic acid (PLA), poly-glycolic acid (PGA), as tracers (e.g. radioisotopes), contrasting agents (e.g. imaging dyes), aptamers, avimers, peptides, nucleic acids, modified polysaccharide coatings, drugs (chemotherapy drugs), and recombinant antibodies or antibody-based moieties.

Aspects of the present specification provide a synthetic molecule having the formula: (molecule X)ₙ-(spacer peptide)₀₋₃₀₀-(tail)-NH₂ for linking with silk molecule or fibroin, wherein “n” is a whole integer ranging from 1-30, and wherein the amino acid residues of the spacer ranges from 0-300. Examples of such synthetic molecule capable for linking to silk molecule or fibroin are: GRGDIPASSKG₄SRL₆R—NH₂ (SEQ ID NO: 1), Ac-GdRGDIPASSKG₄SdRL₆dR-NH₂ (SEQ ID NO: 2), (VEGF)-(VEGF)-GSPGISGGGGGILEKLLLLLK-NH₂ (SEQ ID NO: 26), (HIV-C-peptide)-3-GSPGISGGGGGILEKLALWLLR-NH₂ (SEQ ID NO: 27), (taxol)₂-GS PGISGGGGGILERLLLLR-NH₂ (SEQ ID NO: 28), and (EPO)₂-GSPGISGGGGGILERLLWLLR-NH₂ (SEQ ID NO: 29). When used in the context of the silk materials described herein, the synthetic molecule of SEQ ID NO: 1 enables better tissue attachment of the construct in vivo, the synthetic molecule of SEQ ID NO: 26 can promote blood vessel generation (neo-angiogenesis) in tissue engineered constructs, the synthetic molecule of SEQ ID NO: 28 can provide a slow release anti-HIV medication in the form of a transdermal delivery patch, the synthetic molecule of SEQ ID. NO: 28 can provide sustained dosage of anti-cancer drug in vivo, and the synthetic molecule of SEQ ID NO: 29 can provide a slow release EPO during cancer chemotherapy.

Aspects of the present specification disclose, in part, a hydrogel comprising a synthetic molecule having the formula: (molecule X)ₙ-(spacer peptide)₀₋₃₀₀-(tail)-NH₂ or a synthetic molecule having the formula: (molecule X)ₙ-(spacer peptide)₀₋₃₀₀-(tail)-NH₂ and an amphiphilic peptide. In one embodiment, the amphiphilic peptide is 23RGD. In one embodiment, the present specification provides a method of conjugating a molecule X to a silk molecule or fibroin comprising mixing a synthetic molecule having the formula: (molecule X)ₙ-(spacer peptide)₀₋₃₀₀-(tail)-NH₂ with a silk molecule or fibroin or silk solution. Conjugation of individual peptide can be effected by a linkage via the N-terminal or the C-terminal of the peptide, resulting in an N-linked peptide oligomer or a C-linked peptide oligomer, respectively.

Methods of peptide synthesis are known to one skilled in the art, e.g., exclusively solid phase techniques, partial solid-phase techniques, fragment condensation or classical solution couplings. For example, the disclosed peptides can be synthesized by the solid phase method using standard methods based on either t-butyloxycarbonyl (BOC) or 9-fluorenylmethoxy-carbonyl (FMOC) protecting groups. See, e.g., Fields et al. in Synthetic Peptides: A Users Guide, W. M. Freeman & Company, New York, N.Y., pp. 77-183 (1992); “Solid-Phase Synthesis”, Stewart & Young, Freemen & Company, San Francisco, 1969; and U.S. Pat. No. 4,105,603. Classical solution synthesis is described in “Methoden der Organischen Chemic (Houben-Weyl): Synthese von Peptiden”, E. Wunsch (editor) (1974) Georg Thieme Verlag, Stuttgart West Germany. The fragment condensation method of synthesis is disclosed in U.S. Pat. No. 3,972,859. Other syntheses methods are disclosed in U.S. Pat. No. 3,842,067, U.S. Pat. No. 3,872,925, issued Jan. 28, 1975, Merrifield B, Protein Science (1996), 5: 1947-1951; The chemical synthesis of proteins; Mutter M, Int J Pept Protein Res 1979 March; 13 (3): 274-7 Studies on the coupling rates in liquid-phase peptide synthesis using competition experiments; and Solid Phase Peptide Synthesis in the series Methods in Enzymology (Fields, G. B. (1997) Solid-Phase Peptide Synthesis. Academic Press, San Diego). The foregoing disclosures are incorporated herein by reference. Molecular DNA methods can also be used. The coding sequence of the short spacer can be constructed be annealing a complementary pair of primers. One of skill in the art can design and synthesize oligonucleotides that will code for the selected spacer.

Methods of linking peptides are also known in the art. The physical linking of the individual isolated peptides into oligomeric peptides as set forth herein, can be effected by chemical conjugation procedures well known in the art, such as by creating peptide linkages, use of condensation agents, and by employing well known bifunctional cross-linking reagents. The conjugation may be direct, which includes linkages not involving any intervening group, e.g., direct peptide linkages, or indirect, wherein the linkage contains an intervening moiety, such as a protein or peptide, e.g., plasma albumin, or other spacer molecule. For example, the linkage may be via a heterobifunctional or homobifunctional cross-linker, e.g., carbodiimide, glutaraldehyde, N-succinimidyl 3-(2-pyridydithio)propionate (SPDP) and derivatives, bis-maleimide, 4-(N-maleimidomethyl)cyclohexane-1-carboxylate, and the like.

Cross-linking can also be accomplished without exogenous cross-linkers by using reactive groups on the molecules being conjugated. Methods for chemically cross-linking peptide molecules are known in the art, and a number of hetero- and homobifunctional agents are described in, e.g., U.S. Pat. Nos. 4,355,023, 4,657,853, 4,676,980, 4,925,921, and 4,970,156, and Immuno Technology Catalogue and Handbook, Pierce Chemical Co. (1989), each of which is incorporated herein by reference. Such conjugation, including cross-linking, should be performed so as not to substantially affect the desired function of the peptide or entity conjugated thereto, including therapeutic agents, and moieties capable of binding substances of interest.

It is apparent to a skilled artisan that alternative agents can be used to link peptides. For example homobifunctional crosslinker such as disuccinimidyl-suberimidate-dihydrochloride; dimethyl-adipimidate-dihydrochloride; 1,5,-2,4-dinitrobenezene or heterobifunctional crosslinkers such as N-hydroxysuccinimidyl 2,3-dibromopropionate; 1-ethyl-3-[3-dimethylaminopropyl]carbodiimide hydrochloride; and succinimidyl-4-[n-maleimidomethyl]-cyclohexane-1-carboxylate.

The drug delivery platforms disclosed herein can further comprise other polymers in addition to silk fibroin, amphiphilic peptides and the synthetic molecules disclosed herein. Such polymers are generally included to prolong the release of certain growth factors or cytokines and to stabilize the functionality. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked.

Suitable polymers include, without limitation, alginates, aliphatic polyesters, polyalkylene oxalates, polyamides, polyamidoesters, polyanhydrides, polycarbonates, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. Al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. patent application Ser. No. 12/764,039, filed on Apr. 20, 2010; each of which is incorporated by reference in its entirety.

In aspects of this embodiment, a polymer included in the disclosed biomaterials is a polypeptide such as, e.g., silk fibroin, keratin, or collagen. In other aspects of this embodiment, a polymer included in the disclosed biomaterials is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer included in the disclosed biomaterials is a polyester such as, e.g., D-lactic acid, L-lactic acid, racemic lactic acid, glycolic acid, caprolactone, and combinations thereof.

One of ordinary skill in the art appreciates that the selection of a suitable amounts of silk fibroin, amphiphilic peptide, and other polymer for forming a disclosed drug delivery platform depends on several factors. The more relevant factors in this selection, include, without limitation, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, compatibility of other polymer with compound, and patient tolerance.

Aspects of the present specification disclose, in part, a drug delivery platform comprising a biomaterial comprising a silk fibroin, an amphiphilic peptide, and/or another polymer. A drug delivery platform disclosed herein is typically a biodegradable, bioerodible, and/or bioresorbable.

In an embodiment, a silk fibroin biomaterial disclosed herein has a protein structure that makes the biomaterial resist biodegradation. In aspects of this embodiment, a biomaterial is resistant to biodegradation for, e.g., about 10 days, about 20 days, about 30 days, about 40 days, about 50 days, about 60 days, about 70 days, about 80 days, or about 90 days. In other aspects of this embodiment, a biomaterial is resistant to biodegradation for, e.g., at least 10 days, at least 20 days, at least 30 days, at least 40 days, at least 50 days, at least 60 days, at least 70 days, at least 80 days, or at least 90 days. In yet other aspects of this embodiment, a biomaterial is resistant to biodegradation for, e.g., about 10 days to about 30 days, about 20 days to about 50 days, about 40 days to about 60 days, about 50 days to about 80 days, or about 60 days to about 90 days.

In an embodiment, a silk fibroin biomaterial disclosed herein has a protein structure that makes the biomaterial resist bioerosion. In aspects of this embodiment, a biomaterial is resistant to bioerosion for, e.g., about 10 days, about 20 days, about 30 days, about 40 days, about 50 days, about 60 days, about 70 days, about 80 days, or about 90 days. In other aspects of this embodiment, a biomaterial is resistant to bioerosion for, e.g., at least 10 days, at least 20 days, at least 30 days, at least 40 days, at least 50 days, at least 60 days, at least 70 days, at least 80 days, or at least 90 days. In yet other aspects of this embodiment, a biomaterial is resistant to bioerosion for, e.g., about 10 days to about 30 days, about 20 days to about 50 days, about 40 days to about 60 days, about 50 days to about 80 days, or about 60 days to about 90 days.

In an embodiment, a silk fibroin biomaterial disclosed herein has a protein structure that makes the biomaterial resist bioresorption. In aspects of this embodiment, a biomaterial is resistant to bioresorption for, e.g., about 10 days, about 20 days, about 30 days, about 40 days, about 50 days, about 60 days, about 70 days, about 80 days, or about 90 days. In other aspects of this embodiment, a biomaterial is resistant to bioresorption for, e.g., at least 10 days, at least 20 days, at least 30 days, at least 40 days, at least 50 days, at least 60 days, at least 70 days, at least 80 days, or at least 90 days. In yet other aspects of this embodiment, a biomaterial is resistant to bioresorption for, e.g., about 10 days to about 30 days, about 20 days to about 50 days, about 40 days to about 60 days, about 50 days to about 80 days, or about 60 days to about 90 days.

In yet another embodiment, a silk fibroin biomaterial disclosed herein has a protein structure that substantially includes β-turn and β-strand regions. In aspects of this embodiment, a biomaterial has a protein structure including, e.g., about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of this embodiment, a biomaterial has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of this embodiment, a biomaterial has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions.

In yet another embodiment, a silk fibroin biomaterial disclosed herein has a protein structure that is substantially-free of α-helix and random coil regions. In aspects of this embodiment, a biomaterial has a protein structure including, e.g., about 5% α-helix and random coil regions, about 10% α-helix and random coil regions, about 15% α-helix and random coil regions, about 20% α-helix and random coil regions, about 25% α-helix and random coil regions, about 30% α-helix and random coil regions, about 35% α-helix and random coil regions, about 40% α-helix and random coil regions, about 45% α-helix and random coil regions, or about 50% α-helix and random coil regions. In other aspects of this embodiment, a biomaterial has a protein structure including, e.g., at most 5% α-helix and random coil regions, at most 10% α-helix and random coil regions, at most 15% α-helix and random coil regions, at most 20% α-helix and random coil regions, at most 25% α-helix and random coil regions, at most 30% α-helix and random coil regions, at most 35% α-helix and random coil regions, at most 40% α-helix and random coil regions, at most 45% α-helix and random coil regions, or at most 50% α-helix and random coil regions. In yet other aspects of this embodiment, a biomaterial has a protein structure including, e.g., about 5% to about 10% α-helix and random coil regions, about 5% to about 15% α-helix and random coil regions, about 5% to about 20% α-helix and random coil regions, about 5% to about 25% α-helix and random coil regions, about 5% to about 30% α-helix and random coil regions, about 5% to about 40% α-helix and random coil regions, about 5% to about 50% α-helix and random coil regions, about 10% to about 20% α-helix and random coil regions, about 10% to about 30% α-helix and random coil regions, about 15% to about 25% α-helix and random coil regions, about 15% to about 30% α-helix and random coil regions, or about 15% to about 35% α-helix and random coil regions.

Aspects of the present specification provide, in part, a silk fibroin biomaterial having a hardness. Hardness refers to various properties of an object in the solid phase that gives it high resistance to various kinds of shape change when force is applied. Hardness is measured using a durometer and is a unitless value that ranges from zero to 100. The ability or inability of a biomaterial to be easily compressed will affect its suitability for application in different tissue replacement roles, i.e., mechanical compliance as bone, fat, connective tissue. Hardness will also affect the ability of a biomaterial to be effectively comminuted, the reason being that a hard material may be more easily and consistently comminuted. Hardness will also affect extrudability, as a soft material may be more readily able to be slightly compressed during injection to pack with other particles or change shape to pass through a syringe barrel or needle.

In an embodiment, a silk fibroin biomaterial exhibits low hardness. In aspects of this embodiment, a silk fibroin biomaterial exhibits a hardness of, e.g., about 5, about 10, about 15, about 20, about 25, about 30, or about 35. In other aspects of this embodiment, a silk fibroin biomaterial exhibits a hardness of, e.g., at most 5, at most 10, at most 15, at most 20, at most 25, at most 30, or at most 35. In yet other aspects of this embodiment, a silk fibroin biomaterial exhibits a hardness of, e.g., about 5 to about 35, about 10 to about 35, about 15 to about 35, about 20 to about 35, or about 25 to about 35, about 5 to about 40, about 10 to about 40, about 15 to about 40, about 20 to about 40, about 25 to about 40, or about 30 to about 40

In an embodiment, a silk fibroin biomaterial exhibits medium hardness. In aspects of this embodiment, a silk fibroin biomaterial exhibits a hardness of, e.g., about 40, about 45, about 50, about 55, or about 60. In other aspects of this embodiment, a silk fibroin biomaterial exhibits a hardness of, e.g., at least 40, at least 45, at least 50, at least 55, or at least 60. In yet other aspects of this embodiment, a silk fibroin biomaterial exhibits a hardness of, e.g., at most 40, at most 45, at most 50, at most 55, or at most 60. In still other aspects of this embodiment, a silk fibroin biomaterial exhibits a hardness of, e.g., about 35 to about 60, about 35 to about 55, about 35 to about 50, about 35 to about 45, about 40 to about 60, about 45 to about 60, about 50 to about 60, about 55 to about 60, about 40 to about 65, about 45 to about 65, about 50 to about 65, about 55 to about 65.

In another embodiment, a silk fibroin biomaterial exhibits high hardness. In aspects of this embodiment, a silk fibroin biomaterial exhibits a hardness of, e.g., about 65, about 70, about 75, about 80, about 85, about 90, about 95, or about 100. In other aspects of this embodiment, a silk fibroin biomaterial exhibits a hardness of, e.g., at least 65, at least 70, at least 75, at least 80, at least 85, at least 90, at least 95, or at least 100. In yet other aspects of this embodiment, a silk fibroin biomaterial exhibits a hardness of, e.g., about 65 to about 100, about 70 to about 100, about 75 to about 100, about 80 to about 100, about 85 to about 100, about 90 to about 100, about 65 to about 75, about 65 to about 80, about 65 to about 85, about 65 to about 90, about 65 to about 95, about 60 to about 75, about 60 to about 80, about 60 to about 85, about 60 to about 90, or about 60 to about 95.

In an embodiment, a silk fibroin biomaterial exhibits high resistant to deformation. In aspects of this embodiment, a silk fibroin biomaterial exhibits resistant to deformation of, e.g., about 100%, about 99%, about 98%, about 97%, about 96%, about 95%, about 94%, about 93%, about 92%, about 91%, about 90%, about 89%, about 88%, about 87%, about 86%, or about 85%. In other aspects of this embodiment, a silk fibroin biomaterial exhibits resistant to deformation of, e.g., at least 99%, at least 98%, at least 97%, at least 96%, at least 95%, at least 94%, at least 93%, at least 92%, at least 91%, at least 90%, at least 89%, at least 88%, at least 87%, at least 86%, or at least 85%. In yet other aspects of this embodiment, a silk fibroin biomaterial exhibits resistant to deformation of, e.g., at most 99%, at most 98%, at most 97%, at most 96%, at most 95%, at most 94%, at most 93%, at most 92%, at most 91%, at most 90%, at most 89%, at most 88%, at most 87%, at most 86%, or at most 85%. In still aspects of this embodiment, a silk fibroin biomaterial exhibits resistant to deformation of, e.g., about 85% to about 100%, about 87% to about 100%, about 90% to about 100%, about 93% to about 100%, about 95% to about 100%, or about 97% to about 100%.

A silk fibroin biomaterial exhibits an elastic modulus. Elastic modulus, or modulus of elasticity, refers to the ability of a biomaterial material to resists deformation, or, conversely, an object's tendency to be non-permanently deformed when a force is applied to it. The elastic modulus of an object is defined as the slope of its stress-strain curve in the elastic deformation region: λ=stress/strain, where λ is the elastic modulus in Pascal's; stress is the force causing the deformation divided by the area to which the force is applied; and strain is the ratio of the change caused by the stress to the original state of the object. Specifying how stresses are to be measured, including directions, allows for many types of elastic moduli to be defined. The three primary elastic moduli are tensile modulus, shear modulus, and bulk modulus.

Tensile modulus (E) or Young's modulus is an objects response to linear strain, or the tendency of an object to deform along an axis when opposing forces are applied along that axis. It is defined as the ratio of tensile stress to tensile strain. It is often referred to simply as the elastic modulus. The shear modulus or modulus of rigidity refers to an object's tendency to shear (the deformation of shape at constant volume) when acted upon by opposing forces. It is defined as shear stress over shear strain. The shear modulus is part of the derivation of viscosity. The shear modulus is concerned with the deformation of a solid when it experiences a force parallel to one of its surfaces while its opposite face experiences an opposing force (such as friction). The bulk modulus (K) describes volumetric elasticity or an object's resistance to uniform compression, and is the tendency of an object to deform in all directions when uniformly loaded in all directions. It is defined as volumetric stress over volumetric strain, and is the inverse of compressibility. The bulk modulus is an extension of Young's modulus to three dimensions.

In another embodiment, a silk fibroin biomaterial exhibits a tensile modulus. In aspects of this embodiment, a silk fibroin biomaterial exhibits a tensile modulus of, e.g., about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa. In other aspects of this embodiment, a silk fibroin biomaterial exhibits a tensile modulus of, e.g., at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa In yet other aspects of this embodiment, a silk fibroin biomaterial exhibits a tensile modulus of, e.g., about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa.

In another embodiment, a silk fibroin biomaterial exhibits shear modulus. In aspects of this embodiment, a silk fibroin biomaterial exhibits a shear modulus of, e.g., about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa, about 750 MPa, about 1 GPa, about 5 GPa, about 10 GPa, about 15 GPa, about 20 GPa, about 25 GPa, or about 30 GPa. In other aspects of this embodiment, a silk fibroin biomaterial exhibits a shear modulus of, e.g., at least 1 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa, at least 750 MPa, at least 1 GPa, at least 5 GPa, at least 10 GPa, at least 15 GPa, at least 20 GPa, at least 25 GPa, or at least 30 GPa In yet other aspects of this embodiment, a silk fibroin biomaterial exhibits a shear modulus of, e.g., about 1 MPa to about 30 MPa, about 10 MPa to about 50 MPa, about 25 MPa to about 75 MPa, about 50 MPa to about 100 MPa, about 100 MPa to about 300 MPa, about 200 MPa to about 400 MPa, about 300 MPa to about 500 MPa, about 100 MPa to about 500 MPa, about 250 MPa to about 750 MPa, about 500 MPa to about 1 GPa, about 1 GPa to about 30 GPa, about 10 GPa to about 30 GPa.

In another embodiment, a silk fibroin biomaterial exhibits a bulk modulus. In aspects of this embodiment, a silk fibroin biomaterial exhibits a bulk modulus of, e.g., about 5 GPa, about 6 GPa, about 7 GPa, about 8 GPa, about 9 GPa, about 10 GPa, about 15 GPa, about 20 GPa, about 25 GPa, about 30 GPa, about 35 GPa, about 40 GPa, about 45 GPa, about 50 GPa, about 60 GPa, about 70 GPa, about 80 GPa, about 90 GPa, about 100 GPa. In other aspects of this embodiment, a silk fibroin biomaterial exhibits a bulk modulus of, e.g., at least 5 GPa, at least 6 GPa, at least 7 GPa, at least 8 GPa, at least 9 GPa, at least 10 GPa, at least 15 GPa, at least 20 GPa, at least 25 GPa, at least 30 GPa, at least 35 GPa, at least 40 GPa, at least 45 GPa, at least 50 GPa, at least 60 GPa, at least 70 GPa, at least 80 GPa, at least 90 GPa, at least 100 GPa. In yet other aspects of this embodiment, a silk fibroin biomaterial exhibits a bulk modulus of, e.g., about 5 GPa to about 50 GPa, about 5 GPa to about 100 GPa, about 10 GPa to about 50 GPa, about 10 GPa to about 100 GPa, or about 50 GPa to about 100 GPa.

A silk fibroin biomaterial exhibits high tensile strength. Tensile strength has three different definitional points of stress maxima. Yield strength refers to the stress at which material strain changes from elastic deformation to plastic deformation, causing it to deform permanently. Ultimate strength refers to the maximum stress a material can withstand when subjected to tension, compression or shearing. It is the maximum stress on the stress-strain curve. Breaking strength refers to the stress coordinate on the stress-strain curve at the point of rupture, or when the material pulls apart.

In another embodiment, a silk fibroin biomaterial exhibits high yield strength relative to other polymer classes. In aspects of this embodiment, an elastomer matrix defining an array of interconnected pores exhibits a yield strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of this embodiment, a silk fibroin biomaterial exhibits a yield strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of this embodiment, a silk fibroin biomaterial exhibits a yield strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, a silk fibroin biomaterial exhibits a yield strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

In another embodiment, a silk fibroin biomaterial exhibits high ultimate strength. In aspects of this embodiment, a silk fibroin biomaterial exhibits an ultimate strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of this embodiment, a silk fibroin biomaterial exhibits an ultimate strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of this embodiment, a silk fibroin biomaterial exhibits an ultimate strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, a silk fibroin biomaterial exhibits an ultimate strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

In another embodiment, a silk fibroin biomaterial exhibits high breaking strength. In aspects of this embodiment, a silk fibroin biomaterial exhibits a breaking strength of, e.g., about 0.1 MPa, about 0.5 MPa, about 1 MPa, about 5 MPa, about 10 MPa, about 20 MPa, about 30 MPa, about 40 MPa, about 50 MPa, about 60 MPa, about 70 MPa, about 80 MPa, about 90 MPa, about 100 MPa, about 200 MPa, about 300 MPa, about 400 MPa, about 500 MPa. In other aspects of this embodiment, a silk fibroin biomaterial exhibits a breaking strength of, e.g., at least 0.1 MPa, at least 0.5 MPa, at least 1 MPa, at least 5 MPa, at least 10 MPa, at least 20 MPa, at least 30 MPa, at least 40 MPa, at least 50 MPa, at least 60 MPa, at least 70 MPa, at least 80 MPa, at least 90 MPa, at least 100 MPa, at least 200 MPa, at least 300 MPa, at least 400 MPa, at least 500 MPa. In yet other aspects of this embodiment, a silk fibroin biomaterial exhibits a breaking strength of, e.g., at most 1 MPa, at most 5 MPa, at most 10 MPa, at most 20 MPa, at most 30 MPa, at most 40 MPa, at most 50 MPa, at most 60 MPa, at most 70 MPa, at most 80 MPa, at most 90 MPa, at most 100 MPa, at most 200 MPa, at most 300 MPa, at most 400 MPa, at most 500 MPa, at most 600 MPa, at most 700 MPa, at most 800 MPa, at most 900 MPa, at most 1000 MPa, at most 1500 MPa, or at most 2000 MPa. In still other aspects of this embodiment, a silk fibroin biomaterial exhibits a breaking strength of, e.g., about 1 MPa to about 50 MPa, about 1 MPa to about 60 MPa, about 1 MPa to about 70 MPa, about 1 MPa to about 80 MPa, about 1 MPa to about 90 MPa, about 1 MPa to about 100 MPa, about 10 MPa to about 50 MPa, about 10 MPa to about 60 MPa, about 10 MPa to about 70 MPa, about 10 MPa to about 80 MPa, about 10 MPa to about 90 MPa, about 10 MPa to about 100 MPa, about 10 MPa to about 200 MPa, about 10 MPa to about 300 MPa, or about 100 MPa to about 300 MPa.

Aspects of the present specification provide, in part, a silk fibroin biomaterial having a transparency and/or translucency. Transparency (pellucidity or diaphaneity) is the physical property of allowing light to pass through a material, whereas translucency (translucence or translucidity) only allows light to pass through diffusely. The opposite property is opacity. Transparent materials are clear, while translucent ones cannot be seen through clearly. The silk fibroin biomaterials disclosed herein may exhibit optical properties such as transparency and translucency. In certain cases, e.g., superficial line filling, it would be an advantage to have an opaque biomaterial. In other cases such as development of a lens or a “humor” for filling the eye, it would be an advantage to have a translucent biomaterial. These properties could be modified by affecting the structural distribution of the biomaterial material. Factors used to control a biomaterial's optical properties include, without limitation, silk fibroin concentration, crystallinity, and homogeneity.

When light encounters a material, it can interact with it in several different ways. These interactions depend on the nature of the light (wavelength, frequency, energy, etc.) and the nature of the material. Light waves interact with an object by some combination of reflection, and transmittance with refraction. As such, an optically transparent material allows much of the light that falls on it to be transmitted, with little light being reflected. Materials which do not allow the transmission of light are called optically opaque or simply opaque.

In an embodiment, a silk fibroin biomaterial is optically transparent. In aspects of this embodiment, a silk fibroin biomaterial transmits, e.g., about 75% of the light, about 80% of the light, about 85% of the light, about 90% of the light, about 95% of the light, or about 100% of the light. In other aspects of this embodiment, a silk fibroin biomaterial transmits, e.g., at least 75% of the light, at least 80% of the light, at least 85% of the light, at least 90% of the light, or at least 95% of the light. In yet other aspects of this embodiment, a silk fibroin biomaterial transmits, e.g., about 75% to about 100% of the light, about 80% to about 100% of the light, about 85% to about 100% of the light, about 90% to about 100% of the light, or about 95% to about 100% of the light.

In another embodiment, a silk fibroin biomaterial is optically opaque. In aspects of this embodiment, a silk fibroin biomaterial transmits, e.g., about 5% of the light, about 10% of the light, about 15% of the light, about 20% of the light, about 25% of the light, about 30% of the light, about 35% of the light, about 40% of the light, about 45% of the light, about 50% of the light, about 55% of the light, about 60% of the light, about 65% of the light, or about 70% of the light. In other aspects of this embodiment, a silk fibroin biomaterial transmits, e.g., at most 5% of the light, at most 10% of the light, at most 15% of the light, at most 20% of the light, at most 25% of the light, at most 30% of the light, at most 35% of the light, at most 40% of the light, at most 45% of the light, at most 50% of the light, at most 55% of the light, at most 60% of the light, at most 65% of the light, at most 70% of the light, or at most 75% of the light. In other aspects of this embodiment, a silk fibroin biomaterial transmits, e.g., about 5% to about 15%, about 5% to about 20%, about 5% to about 25%, about 5% to about 30%, about 5% to about 35%, about 5% to about 40%, about 5% to about 45%, about 5% to about 50%, about 5% to about 55%, about 5% to about 60%, about 5% to about 65%, about 5% to about 70%, about 5% to about 75%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 25% to about 35%, about 25% to about 40%, about 25% to about 45%, about 25% to about 50%, about 25% to about 55%, about 25% to about 60%, about 25% to about 65%, about 25% to about 70%, or about 25% to about 75%.

In an embodiment, a silk fibroin biomaterial is optically translucent. In aspects of this embodiment, a silk fibroin biomaterial diffusely transmits, e.g., about 75% of the light, about 80% of the light, about 85% of the light, about 90% of the light, about 95% of the light, or about 100% of the light. In other aspects of this embodiment, a silk fibroin biomaterial diffusely transmits, e.g., at least 75% of the light, at least 80% of the light, at least 85% of the light, at least 90% of the light, or at least 95% of the light. In yet other aspects of this embodiment, a silk fibroin biomaterial diffusely transmits, e.g., about 75% to about 100% of the light, about 80% to about 100% of the light, about 85% to about 100% of the light, about 90% to about 100% of the light, or about 95% to about 100% of the light.

Aspects of the present specification provide, in part, a biomaterial comprising a compound having the structure of formula I

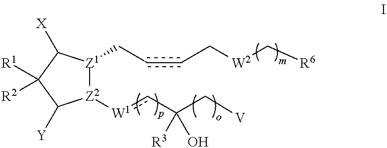

wherein each dashed line represents the presence or absence of a bond; R¹, R² and R³ are each independently selected from H or C₁-C₆ alkyl; R⁶ is CO₂H, CO₂R⁷, CON(R⁷)₂, CONHCH₂CH₂OH, CON(CH₂CH₂OH)₂, CH₂OR⁷, P(O)(OR⁷)₂, or

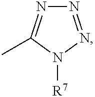

a pharmaceutically acceptable salt thereof, or a pharmaceutically acceptable amine salt thereof; R⁷ is H, C₁-C₆ alkyl or C₂-C₆ alkenyl; X and Y are each independently selected from H, OH, ═O, Cl, Br, I, or CF₃; Z¹ and Z² are each independently selected from CH or N; W¹ and W² are each independently selected from CH, CH₂, aryl or substituted aryl, heteroaryl, substituted heteroaryl; m is 0 to 6; o is 0 to 4; p is 0 or 1; and V is C₁-C₆ alkyl, aryl, substituted aryl, heteroaryl, or substituted heteroaryl.

The method of preparing the compounds represented by formula I can be found in, e.g., Donde, et el., 10,10-Dialkyl Prostanoic Acid Derivatives as Agents for Lowering Intraocular Pressure, U.S. Pat. No. 6,875,787; Donde, et el., 10,10-Dialkyl Prostanoic Acid Derivatives as Agents for Lowering Intraocular Pressure, U.S. Patent Publication 2004/0235958; Donde, et al., Treatment of Inflammatory Bowel Disease, U.S. Patent Publication 2005/0164992, each of which is hereby incorporated by reference in its entirety.

Unless specific definitions are provided, the nomenclatures used in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, and formulation. Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

In all of the disclosed structures, straight lines represent bonds, and where there is no symbol for the atoms between the bonds, the appropriate carbon-containing radical is to be inferred.

As used herein, the term “alkenyl” refers to a functional group comprising a straight-chain or branched-chain hydrocarbon containing from 2 to 20 carbon atoms and having one or more carbon-carbon double bonds and not having any cyclic structure. An alkenyl group may be optionally substituted as defined herein. Examples of alkenyl groups include, without limitation, ethenyl, propenyl, 2-methylpropenyl, butenyl, 1,4-butadienyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, and the like.

As used herein, the term “alkyl” refers to a functional group comprising a straight-chain or branched-chain hydrocarbon containing from 1 to 20 carbon atoms linked exclusively by single bonds and not having any cyclic structure. An alkyl group may be optionally substituted as defined herein. Examples of alkyl groups includes, without limitation methyl, ethyl, n-propyl, isopropyl, n-butyl, isobutyl, sec-butyl, tert-butyl, pentyl, iso-amyl, hexyl, heptyl, octyl, noyl, decyl, undecyl, dodecyl tridecyl, tetradecyl, pentadecyl, hexadecyl, heptadecyl, octadecyl, nonadecyl, eicosyl, and the like.

As used herein, the term “heteroalkyl” refers to a functional group comprising a straight-chain or branched-chain hydrocarbon containing from 1 to 20 atoms linked exclusively by single bonds, where at least one atom in the chain is a carbon and at least one atom in the chain is O, S, N, or any combination thereof. The heteroalkyl group can be fully saturated or contain from 1 to 3 degrees of unsaturation. The non-carbon atoms can be at any interior position of the heteroalkyl group, and up to two non-carbon atoms may be consecutive, such as, e.g., —CH₂—NH—OCH₃. In addition, the non-carbon atoms may optionally be oxidized and the nitrogen may optionally be quaternized.

As used herein, the term “alkynyl” refers to a functional group comprising a straight-chain or branched-chain hydrocarbon containing from 2 to 20 carbon atoms and having one or more carbon-carbon triple bonds and not having any cyclic structure. An alkynyl group may be optionally substituted as defined herein. Examples of alkynyl groups include, without limitation, ethynyl, propynyl, hydroxypropynyl, butynyl, butyn-1-yl, butyn-2-yl, 3-methylbutyn-1-yl, pentynyl, pentyn-1-yl, hexynyl, hexyn-2-yl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, and the like.

As used herein, the term “amine” refers to a molecule comprising an amino group (—NH₂) or derivative thereof. A primary amine has the general formula of RNH₂, a secondary amine has the general formula of NHRR′, a tertiary amine has the general formula of NRR′R″, wherein R, R′, and R″ are an organic moiety or group. As used herein, the term “amino” refers to the functional group —NH₂—. As used herein, the term “aminoalkyl” refers to a functional group comprising an alkyl group attached to the parent molecular moiety through an amino group. An alkylamino group may be a mono- or dialkylated forming group such as, e.g., N-methylamino, N-ethylamino, N,N-dimethylamino, N,N-ethylmethylamino and the like.

As used herein, the term “aryl” or “aryl hydrocarbon” refers to a functional group comprising a substituted or unsubstituted aromatic hydrocarbon with a conjugated cyclic molecular ring structure of 3 to 12 carbon atoms. An aryl group can be monocyclic, bicyclic or polycyclic, and may optionally include one to three additional ring structures, such as, e.g., a cycloalkyl, a cycloalkenyl, a heterocycloalkyl, a heterocycloalkenyl, or a heteroaryl. The term “aryl” includes, without limitation, phenyl (benzenyl), thiophenyl, indolyl, naphthyl, totyl, xylyl, anthracenyl, phenanthryl, azulenyl, biphenyl, naphthalenyl, 1-mMethylnaphthalenyl, acenaphthenyl, acenaphthylenyl, anthracenyl, fluorenyl, phenalenyl, phenanthrenyl, benzo[j]anthracenyl, benzo[c]phenanthrenyl, chrysenyl, fluoranthenyl, pyrenyl, tetracenyl (naphthacenyl), triphenylenyl, anthanthrenyl, benzopyrenyl, benzo[e]pyrenyl, benzo[e]fluoranthenyl, benzo[ghi]perylenyl, benzo[j]fluoranthenyl, benzo[k]fluoranthenyl, corannulenyl, coronenyl, dicoronylenyl, helicenyl, heptacenyl, hexacenyl, ovalenyl, pentacenyl, picenyl, perylenyl, and tetraphenylenyl. In aspects of this embodiment, an aryl is a 3 carbon aryl, a 4 carbon aryl, a 5 carbon aryl, a 6 carbon aryl, a 7 carbon aryl, a 8 carbon aryl, a 9 carbon aryl, a 10 carbon aryl, a 11 carbon aryl, or a 12 carbon aryl.

As used herein, the term “lower aryl” refers to a functional group comprising a substituted or unsubstituted aromatic hydrocarbon with a conjugated cyclic molecular ring structure of 3 to 6 carbon atoms. Examples of lower aryl groups include, without limitation, phenyl and naphthyl. In aspects of this embodiment, a lower aryl is a 3 carbon aryl, a 4 carbon aryl, a 5 carbon aryl, or a 6 carbon aryl. In other aspects, a lower aryl is a 5 or 6 carbon aryl.

As used herein, the term “heteroaryl” refers to a functional group comprising a substituted or unsubstituted aromatic hydrocarbon with a conjugated cyclic molecular ring structure of 3 to 12 atoms, where at least one atom in the ring structure is a carbon and at least one atom in the ring structure is O, S, N, or any combination thereof. A heteroaryl group can be monocyclic, bicyclic or polycyclic, and may optionally include one to three additional ring structures, such as, e.g., an aryl, a cycloalkyl, a cycloalkenyl, a heterocycloalkyl, or a heterocycloalkenyl. Examples of heteroaryl groups include, without limitation, acridinyl, benzidolyl, benzimidazolyl, benzisoxazolyl, benzodioxinyl, dihydrobenzodioxinyl, benzodioxolyl, 1,3-benzodioxolyl, benzofuryl, benzoisoxazolyl, benzopyranyl, benzothiophenyl, benzo[c]thiophenyl, benzotriazolyl, benzoxadiazolyl, benzoxazolyl, benzothiadiazolyl, benzothiazolyl, benzothienyl, carbazolyl, chromonyl, cinnolinyl, dihydrocinnolinyl, coumarinyl, dibenzofuranyl, furopyridinyl, furyl, indolizinyl, indolyl, dihydroindolyl, imidazolyl, indazolyl, isobenzofuryl, isoindolyl, isoindolinyl, dihydroisoindolyl, isoquinolyl, dihydroisoquinolinyl, isoxazolyl, isothiazolyl, oxazolyl, oxadiazolyl, phenanthrolinyl, phenanthridinyl, purinyl, pyranyl, pyrazinyl, pyrazolyl, pyridyl, pyrimidinyl, pyridazinyl, pyrrolinyl, pyrrolyl, pyrrolopyridinyl, quinolyl, quinoxalinyl, quinazolinyl, tetrahydroquinolinyl, tetrazolopyridazinyl, tetrahydroisoquinolinyl, thiophenyl, thiazolyl, thiadiazolyl, thienopyridinyl, thienyl, thiophenyl, triazolyl, xanthenyl, and the like. In aspects of this embodiment, a heteroaryl is a 3 carbon heteroaryl, a 4 carbon heteroaryl, a 5 carbon heteroaryl, a 6 carbon heteroaryl, a 7 carbon heteroaryl, a 8 carbon heteroaryl, a 9 carbon heteroaryl, a 10 carbon heteroaryl, a 11 carbon heteroaryl, or a 12 carbon heteroaryl.

As used herein, the term “lower heteroaryl” refers to a functional group comprising a monocyclic or bicyclic, substituted or unsubstituted aromatic hydrocarbon with a conjugated cyclic molecular ring structure of 3 to 6 atoms, where at least one atom in the ring structure is a carbon and at least one atom in the ring structure is O, S, N, or any combination thereof. In aspects of this embodiment, a lower heteroaryl is a 3 carbon heteroaryl, a 4 carbon heteroaryl, a 5 carbon heteroaryl, or a 6 carbon heteroaryl. In other aspects, a lower heteroaryl is a 5 or 6 carbon heteroaryl.

As used herein, the term “cycloalkenyl” and “cycloolefin” refers to a functional group comprising a substituted or unsubstituted non-aromatic hydrocarbon with a non-conjugated cyclic molecular ring structure of 3 to 12 carbon atoms having at least one carbon-carbon double bond in the carbon ring structure. A cycloalkenyl group can be monocyclic, bicyclic or polycyclic, and may optionally include one to three additional ring structures, such as, e.g., an aryl, a heteroaryl, a cycloalkyl, a heterocycloalkyl, or a heterocycloalkenyl. Examples of such cycloalkenyl groups include, without limitation, cyclopropene, cyclobutene, 1,3-cyclobutadiene, cyclopentene, 1,3-cyclopentadiene, cyclohexene, 1,3-cyclohexadiene, 1,4-cyclohexadiene, cycloheptene, 1,3-cycloheptadiene, 1,4-cycloheptadiene, and 1,5-cycloheptadiene. In aspects of this embodiment, a cycloalkenyl is a 3 carbon cycloalkenyl, a 4 carbon cycloalkenyl, a 5 carbon cycloalkenyl, a 6 carbon cycloalkenyl, a 7 carbon cycloalkenyl, a 8 carbon cycloalkenyl, a 9 carbon cycloalkenyl, a 10 carbon cycloalkenyl, a 11 carbon cycloalkenyl, or a 12 carbon cycloalkenyl.

As used herein, the term “lower cycloalkenyl” refers to a functional group comprising a monocyclic substituted or unsubstituted non-aromatic hydrocarbon with a non-conjugated cyclic molecular ring structure of 3 to 6 carbon atoms having at least one carbon-carbon double bond in the carbon ring structure. In aspects of this embodiment, a lower cycloalkenyl is a 3 carbon cycloalkenyl, a 4 carbon cycloalkenyl, a 5 carbon cycloalkenyl, or a 6 carbon cycloalkenyl. In other aspects, a lower cycloalkenyl is a 5 or 6 carbon cycloalkenyl.

As used herein, the term “heterocycloalkenyl” refers to a functional group comprising a substituted or unsubstituted non-aromatic hydrocarbon with a non-conjugated cyclic molecular ring structure of 3 to 12 atoms having at least one double bond, where at least one atom in the ring structure is a carbon and at least one atom in the ring structure is O, S, N, or any combination thereof. The heterocycloalkenyl group can be unsaturated, fully saturated or contain from 1 to 3 degrees of unsaturation. A heterocycloalkenyl group can be monocyclic, bicyclic or polycyclic, and may optionally include one to three additional ring structures, such as, e.g., an aryl, a heteroaryl, a cycloalkyl, a heterocycloalkyl, or a cycloalkenyl. In aspects of this embodiment, a heterocycloalkenyl is a 3 carbon heterocycloalkenyl, a 4 carbon heterocycloalkenyl, a 5 carbon heterocycloalkenyl, a 6 carbon heterocycloalkenyl, a 7 carbon heterocycloalkenyl, a 8 carbon heterocycloalkenyl, a 9 carbon heterocycloalkenyl, a 10 carbon heterocycloalkenyl, a 11 carbon heterocycloalkenyl, or a 12 carbon heterocycloalkenyl.

As used herein, the term “lower heterocycloalkenyl” refers to a functional group comprising a monocyclic substituted or unsubstituted non-aromatic hydrocarbon with a non-conjugated cyclic molecular ring structure of 3 to 6 atoms having at least one double bond, where at least one atom in the ring structure is a carbon and at least one atom in the ring structure is O, S, N, or any combination thereof. In aspects of this embodiment, a lower heterocycloalkenyl is a 3 carbon heterocycloalkenyl, a 4 carbon heterocycloalkenyl, a 5 carbon heterocycloalkenyl, or a 6 carbon heterocycloalkenyl. In other aspects, a lower heterocycloalkenyl is a 5 or 6 carbon heterocycloalkenyl.

As used herein, the term “cycloalkyl”, “carbocyclicalkyl”, and “carbocyclealkyl” refers to a functional group comprising a substituted or unsubstituted non-aromatic hydrocarbon with a non-conjugated cyclic molecular ring structure of 3 to 12 carbon atoms linked exclusively with carbon-carbon single bonds in the carbon ring structure. A cycloalkyl group can be monocyclic, bicyclic or polycyclic, and may optionally include one to three additional ring structures, such as, e.g., an aryl, a heteroaryl, a cycloalkenyl, a heterocycloalkyl, or a heterocycloalkenyl. In aspects of this embodiment, a cycloalkyl is a 3 carbon cycloalkyl, a 4 carbon cycloalkyl, a 5 carbon cycloalkyl, a 6 carbon cycloalkyl, a 7 carbon cycloalkyl, a 8 carbon cycloalkyl, a 9 carbon cycloalkyl, a 10 carbon cycloalkyl, a 11 carbon cycloalkyl, or a 12 carbon cycloalkyl.

As used herein, the term “lower cycloalkyl” refers to a functional group comprising a monocyclic substituted or unsubstituted non-aromatic hydrocarbon with a non-conjugated cyclic molecular ring structure of 3 to 6 carbon atoms linked exclusively with carbon-carbon single bonds in the carbon ring structure. Examples of lower cycloalkyl groups include, without limitation, cyclopropyl, cyclobutyl, cyclopentyl, and cyclohexyl. In aspects of this embodiment, a lower cycloalkyl is a 3 carbon cycloalkyl, a 4 carbon cycloalkyl, a 5 carbon cycloalkyl, or a 6 carbon cycloalkyl. In other aspects, a lower cycloalkyl is a 5 or 6 carbon cycloalkyl.

As used herein, the term “heterocycloalkyl”, “heterocyclicalkyl”, and “heterocyclealkyl” refers to a functional group comprising a substituted or unsubstituted non-aromatic hydrocarbon with a non-conjugated cyclic molecular ring structure of 3 to 12 atoms linked exclusively with single bonds in the ring structure, where at least one atom in the ring structure is a carbon and at least one atom in the ring structure is O, S, N, or any combination thereof. The heterocycloalkyl group can be unsaturated, fully saturated or contain from 1 to 3 degrees of unsaturation. A heterocycloalkyl group can be monocyclic, bicyclic or polycyclic, and may optionally include one to three additional ring structures, such as, e.g., an aryl, a heteroaryl a cycloalkyl, a cycloalkenyl, or a heterocycloalkenyl. A heterocycle group may be optionally substituted unless specifically prohibited. Examples of such heterocycloalkyl groups include, without limitation, ariridinyl, azirinyl, diazirinyl, oxiranyl, oxirenyl, dioxiranyl, thiiranyl, thiirenyl, azetidinyl, azetyl, diazetidinyl, oxetanyl, oxetyl, dioxetanyl, dioxetenyl, thietanyl, thietyl, dithietanyl, dithietyl, pyrrolidinyl, pyrrolinyl, pyrrolyl, furanyl, dihydrofuranyl, tetrahydrofuranyl, tetrahydrothiophenyl, dihydrothiophenyl, thiophenyl, imidazolidinyl, pyrazolidinyl, imidazolyl, imidazolinyl, pyrazolyl, pyrazolinyl, oxazolidinyl, isoxazolidinyl, oxazolyl, oxazolinyl, isoxazolyl, isoxazolinyl, thiazolidinyl, isothiazolidinyl, thiazolyl, thiazolinyl, isothiazolyl, isothiazolinyl, dioxolanyl, 1,3-dioxanyl, 1,4-dioxanyl, dioxolanyl, 1,3-dioxolanyl, oxathiolanyl, dithiolanyl, triazolyl, dithiazolyl, furazanyl, oxadiazolyl, thiadiazolyl, tetrazolyl, piperidinyl, tetrahydropyridinyl, pyridinyl, dihydropyridinyl, dihydro[1,3]oxazolo[4,5-b]pyridinyl, pyranyl, tetrahydropyranyl, thianyl, thiopyranyl, piperazinyl, diazinyl, morpholinyl, thiomorpholinyl, oxazinyl, thiazinyl, dithianyl, dioxanyl, dioxinyl, triazinyl, trioxanyl, tetrazinyl, azepanyl, azepinyl, oxepanyl, oxepinyl, thiepanyl, thiepinyl, diazepinyl, thiazepinyl, azocanyl, azocinyl, oxecanyl, thiocanyl, and the like. In aspects of this embodiment, a heterocycloalkyl is a 3 carbon heterocycloalkyl, a 4 carbon heterocycloalkyl, a 5 carbon heterocycloalkyl, a 6 carbon heterocycloalkyl, a 7 carbon heterocycloalkyl, a 8 carbon heterocycloalkyl, a 9 carbon heterocycloalkyl, a 10 carbon heterocycloalkyl, a 11 carbon heterocycloalkyl, or a 12 carbon heterocycloalkyl.

As used herein, the term “lower heterocycloalkyl” refers to a functional group comprising a monocyclic substituted or unsubstituted non-aromatic hydrocarbon with a non-conjugated cyclic molecular ring structure of 3 to 6 atoms linked exclusively with single bonds in the ring structure, where at least one atom in the ring structure is a carbon and at least one atom in the ring structure is O, S, N, or any combination thereof. Lower heterocycloalkyls may be unsaturated. Examples of lower heterocycloalkyls include, without limitation, pyrrolidinyl, imidazolidinyl, pyrazolidinyl, piperidinyl, piperazinyl, and morpholinyl. In aspects of this embodiment, a lower heterocycloalkyl is a 3 carbon heterocycloalkyl, a 4 carbon heterocycloalkyl, a 5 carbon heterocycloalkyl, or a 6 carbon heterocycloalkyl. In other aspects, a lower heterocycloalkyl is a 5 or 6 carbon heterocycloalkyl.

As used herein, the term “functional group” refers to a specific group of atoms within a molecule that are responsible for the characteristic chemical reactions of those molecules. As used herein, the term “lower” refers to a functional group or molecule containing from 1 to 6 carbon atoms, unless otherwise specifically defined. As used herein, the term “unsubstituted” refers to a functional group or molecule that has hydrogen atoms at every position on the parent chain of a hydrocarbon (e.g., —CH₂CH₃). As used herein, the term “substituted” refers to a functional group or molecule that has at least one substituent replacing a hydrogen atom at a position on the parent chain of a hydrocarbon. A substituted group may be fully substituted (e.g., —CF₂CF₃), monosubstituted (e.g., —CH₂CH₂F) or substituted at a level anywhere in between fully substituted and monosubstituted (e.g., —CH₂CH₂F, —CHFCH₂F, —CH₂CHF₂, —CHFCHF₂).

As used herein, the term “substituent” refers to an atom or group of atoms substituted in place of a hydrogen atom on the parent chain of a hydrocarbon. Where substituents are recited without qualification as to substitution, both substituted and unsubstituted forms are encompassed. Examples of substituents include, without limitation, acetyl, acyl, acylamino, acyl halide, alkenyl, alkoxy, alkyl, alkylamino, alkylcarbonyl, alkyloxy, alkyloxo, alkylthio, alkynyl, amidine, amido, amino, aryl, arylamino, arylalkenyl, arylalkoxy, arylalkyl, arylalkynyl, arylalkanoyl, aryloxy, arylthio, azide, azo, benzo, carbamyl, carbonyl, carboxyl, carboxamide, carboxamidine, cyanate, cyano, cycloalkenyl, cycloalkyl, diene, cyclodiene, disulfanyl, enone, halide, halogen, haloalkenyl, haloalkoxy, haloalkyl, heteroalkyl, heteroaryl, heterocycloalkenyl, heterocycloalkyl, hydrazinyl, hydrogen, hydroperoxide, hydroxy, hydroxyalkyl, imide, imine, imino, iminohydroxy, isocyanato, isothiocyanato, isocyanate, isocyanide, isothiocyanate, keto, mercaptyl, nitrite, nitroso, nitro, nitrate, oxo, oxy, oxoalkyl, oxyalkyl, oxime, perhaloalkoxy, perhaloalkyl, peroxy, sulfanyl, sulfhydryl, sulfinyl, sulfonyl, sulfyl, sulfonamido, thioalkyl, thiocarbony, thiocarbamyl, thiocyanate, isothiocyanate, thiocyanato, thioketo, trihalomethanesulfonamido, trihalomethoxy, and/or all lower forms therein.

As used herein, the term “optionally substituted” refers to a functional group or molecule that may be either substituted or unsubstituted. Different sets of optional substituents to a particular moiety may be defined as needed; in these cases, the optional substitution will be as defined, often immediately following the phrase, “optionally substituted with.” An optionally substituted group may be unsubstituted (e.g., —CH₂CH₃), fully substituted (e.g., —CF₂CF₃), monosubstituted (e.g., —CH₂CH₂F) or substituted at a level anywhere in between fully substituted and monosubstituted (e.g., —CH₂CH₂F, —CHFCH₂F, —CH₂ CHF₂, —CHFCHF₂).

In another embodiment, the compound has the structure of formula II,

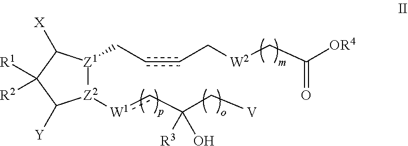

wherein each dashed line represents the presence or absence of a bond; R¹, R² and R³ are each independently selected from H or C₁-C₆ alkyl; R⁴ is H, C₁-C₆ alkyl, C₂-C₆ alkenyl, a pharmaceutically acceptable salt thereof, or an amine thereof; X and Y are each independently selected from H, OH, ═O, Cl, Br, I, or CF₃; Z¹ and Z² are each independently selected from CH or N; W¹ and W² are each independently selected from CH, CH₂, aryl or substituted aryl, heteroaryl, substituted heteroaryl; m is 0 to 4; o is 0 to 4; p is 0 or 1; and V is CH₃, aryl, aryl or substituted aryl, heteroaryl, substituted heteroaryl.

In an aspect of this embodiment, V is

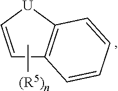

wherein U is C, N, O, or S; R⁵ is halogen, C₁-C₆ alkyl, or C₂-C₆ alkenyl; and n is 0-7; and. In another aspect of this embodiment, U is S; R⁵ is F, Cl, Br, or I; and n is 1, 2, or 3. In yet another aspect of this embodiment W² is thiophene.

In another embodiment, the compound has the structure of formula III,

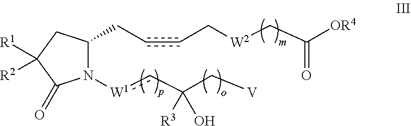

wherein each dashed line represents the presence or absence of a bond; R¹, R² and R³ are each independently selected from H or C₁-C₆ alkyl; R⁴ is H, C₁-C₆ alkyl, C₂-C₆ alkenyl, a pharmaceutically acceptable salt thereof, or an amine thereof; W¹ and W² are each independently selected from CH, CH₂, aryl or substituted aryl, heteroaryl, substituted heteroaryl; m is 0 to 4; o is 0 to 4; p is 0 or 1; and V is CH₃, aryl, aryl or substituted aryl, heteroaryl, substituted heteroaryl.

In yet another embodiment, the compound has the structure of formula IV,

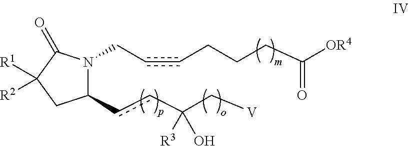

wherein each dashed line represents the presence or absence of a bond; R¹, R² and R³ are each independently selected from H or C₁-C₆ linear alkyl; R⁴ is H, C₁-C₆ alkyl, C₂-C₆ alkenyl, a pharmaceutically acceptable salt thereof, or an amine thereof; m is 0 to 4; o is 0 to 4; p is 0 or 1; and V is CH₃, aryl, aryl or substituted aryl, heteroaryl, substituted heteroaryl.

In still another embodiment, the compound has the structure of formula V,

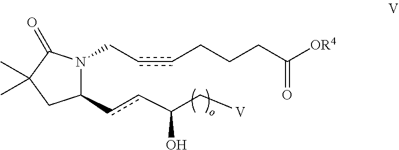

wherein each dashed line represents the presence or absence of a bond; R⁴ is H, C₁-C₆ alkyl, C₂-C₆ alkenyl, a pharmaceutically acceptable salt thereof, or an amine thereof; o is 0 to 4; and V is CH₃, aryl, aryl or substituted aryl, heteroaryl, substituted heteroaryl.

In a further embodiment, the compound has a structure of one of the following compounds listed in Table 1, a pharmaceutically acceptable salt thereof, or an amine thereof

TABLE 1

| | Low R_{f} | High R_{f} |
|---|---|---|
| Structure | diastereomer | diastereomer |
| 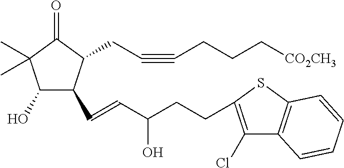 | 34 | 35 |
| 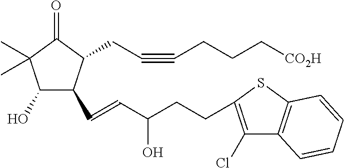 | 36 | 37 |
| 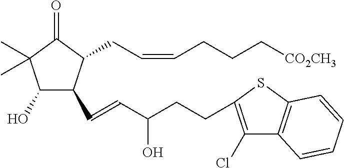 | 38 | 39 |
| 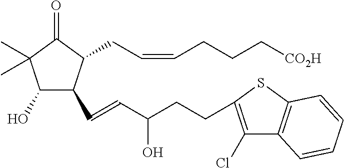 | 40 | 41 |
| 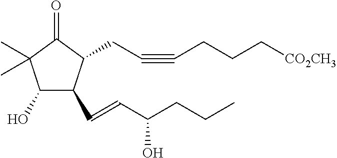 | 42 | |
| 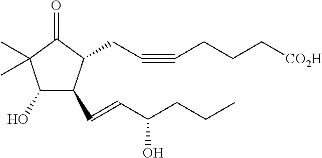 | 43 | |
| 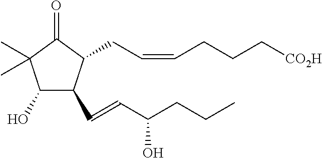 | 44 | |
| 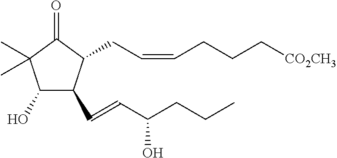 | 45 | |
| 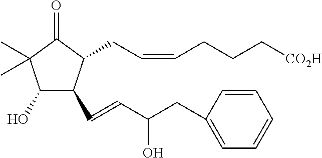 | 46 | 47 |
| 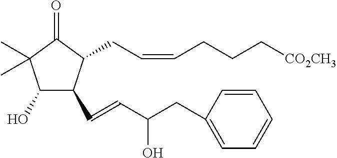 | 48 | 49 |
| 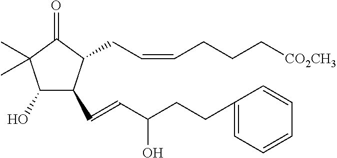 | 50 | 51 |
| 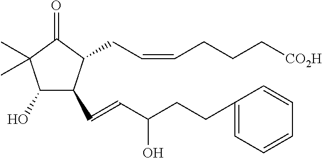 | 52 | 53 |
| 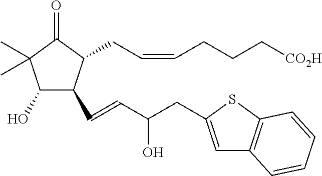 | 54 | 55 |
| 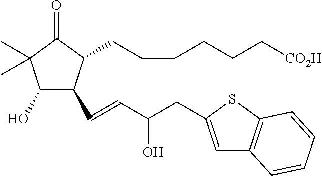 | 56 | 57 |
| 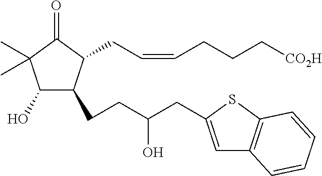 | 58 | 59 |
| 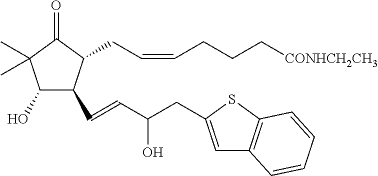 | 60 | 61 |
| 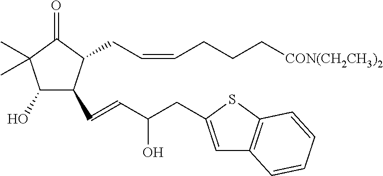 | 62 | 63 |
| 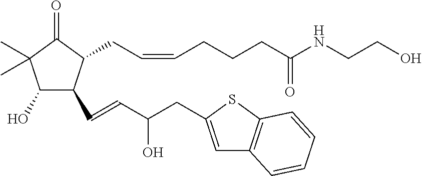 | 64 | 65 |
| 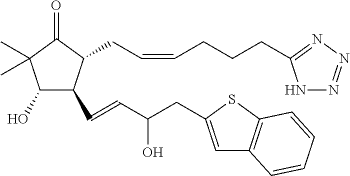 | 66 | 67 |
| 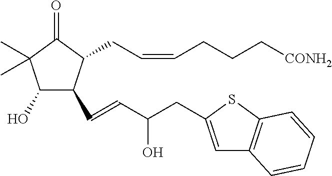 | 68 | 69 |
| 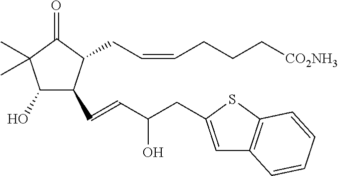 | 70 | 71 |
| 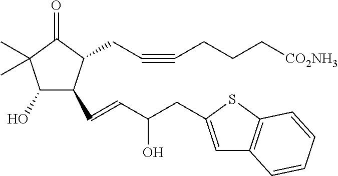 | 72 | 73 |
| 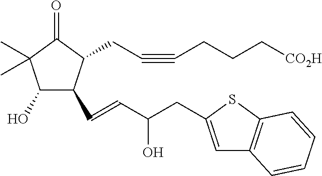 | 74 | 75 |

In a further embodiment, the compound has a structure of compound 1, 2, 3, 4, or 5, a pharmaceutically acceptable salt thereof, or an amine thereof.

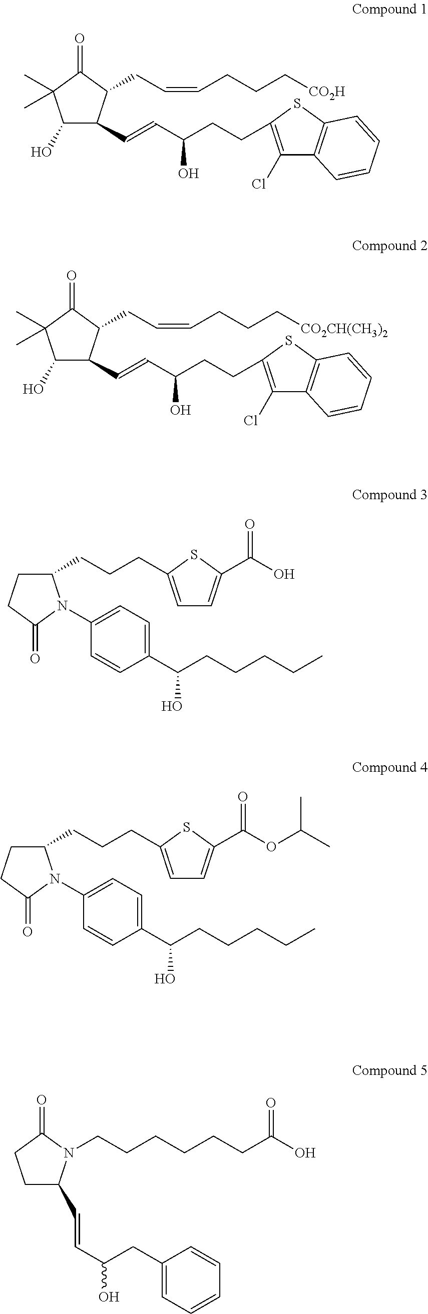

In a yet further embodiment, the compound has a structure of compound 6, a pharmaceutically acceptable salt thereof, or an amine thereof.

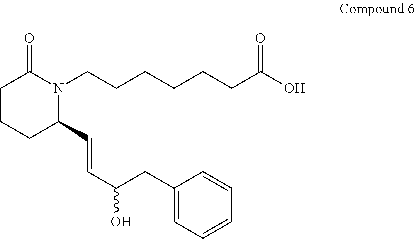

The biomaterials disclosed herein may comprise any number and combination of compounds disclosed herein. For instance, a biomaterial can comprise, e.g., two or more compounds, three or more compounds, four or more compounds or five or more compounds.

A compound disclosed herein may be introduced at any or a combination of several points throughout the silk gel production process. For example, a compound may be added to silk solution or the accelerant phase prior to gelation induction, it could be soaked into the gel during the accelerant rinsing process, or it could be coated onto the bulk gel following rinsing. Gels which are milled and make use of a carrier fluid could also have a compound soaked into the gel following milling, coated onto the gel following milling, or introduced into the carrier fluid before or after blending with the gel material.

A drug delivery platform comprising a biomaterial disclosed herein can be manufactured into any form suitable for delivery of the disclosed compounds. For example, a drug delivery platform comprising a silk fibroin biomaterial and a compound as disclosed herein may be made into a hydrogel, a microsphere, a sheet or film, a porous material like a sponge or foam, filaments, woven filament structures like threads, fibers or mesh, or unwoven filament structures like mats. See, e.g. Kaplin, et al., Silk Boimaterals and Methods of Use Thereof, U.S. Pat. No. 7,674,882; Kaplin, et al., Concentrated Aqueous Silk Fibroin Solution and Use Thereof, U.S. Pat. No. 7,635,755; Kaplan, et al., Silk Microspheres for Encapsulation and Controlled Release, U.S. Patent Application 2010/0028451; Kaplan, et al., Silk-Based Drug Delivery System, U.S. Patent Publication 2008/0085272; Kaplin, et al., Silk Fibroin Materials and Use Thereof, U.S. Patent Publication 2006/0273279; and Kaplin, et al., Silk Biomaterials and Methods of Use Thereof, International Patent Publication WO 2004/001103, each of which is herein incorporated by reference in its entirety.

Silk hydrogels can be prepared by methods disclosed herein, or by methods known in the art, see, e.g., Wang, et al., Method for Silk Fibroin Gelation Using Sonication, U.S. Patent Publication 2010/0178304, which is hereby incorporated by reference in its entirety. Silk fibroin hydrogels can be produced by preparing a solution comprising a concentrated substantially sericin-depleted silk fibroin, mixing the silk fibroin solution with a compound disclosed herein and/or other components disclosed herein, adding a solution comprising a gelation enhancer to the silk fibroin mixture, wherein addition of the enhancer causes polymerization of the depolymerized silk fibroin, thereby generating a hydrogel. Before the sol-gel transition, the concentrated aqueous silk solution can be placed in a mold or form. The resulting hydrogel can then be cut into any shape, using, e.g., a laser, or milled into particles using, e.g., sieves. In addition, the formed silk hydrogel may be further chemically or physically cross-linked to gain altered mechanical properties.

An example of an agent that serves as a gelation enhancer is an alcohol, such as, e.g., ethanol, methanol, and isopropanol; glycerol; and acetone. The volume of added enhancer may vary from about 0% to about 99% of the total volume (i.e., either component may be added to a large excess of the other or in any relative concentration within the interval). The concentration of silk solution used can range from about 1% (w/v) to about 20% (w/v). An amphiphilic peptide disclosed herein may also accelerate gelation under certain circumstances. Such gel may be produced through combination of dissolved silk fibroin solution and an enhancer solution of amphiphilic peptide in alcohol across the silk concentration ranges from about 1% (w/v) to about 20% (w/v), amphiphilic peptide concentration ranges from about 1:100 to 100:1 moles 23RGD:moles silk, and alcohol concentration ranges from about 1% (v/v) to about 99% (v/v) before removal.

A gelation enhancer may also be an agent to improve the biocompatibility. An example agent that both improves gel biocompatibility and serves as a gelation enhancer is an amphiphilic peptide which binds to silk molecules through hydrophobic interactions, such as, e.g., a non-RGD integrin or a RGD motif containing peptide like 23RGD. In other instances, different agents serve these purposes.

Nucleating agents including organic and inorganic species, both soluble and insoluble in an aqueous silk solution intermediate may be used to enhance the gelation process. These may include but are not limited to peptide sequences which bind silk molecules, previously gelled silk, and poorly soluble β-sheet rich structures. A further means of accelerating the gelation process is through the introduction of mechanical excitation. This might be imparted through a shearing device, ultrasound device, or mechanical mixer. It should be borne in mind that any of these factors might conceivably be used in concert with any other or group of others and that the regime would need to be tailored to the desired outcome.

The gelation rate may also be enhanced by increasing the concentration of the depolymerized silk fibroin. This is done by methods including but not limited to dialysis of intermediate silk solution against a buffer incorporating a hygroscopic species such as polyethylene glycol, a lyophilization step, and an evaporation step. Increased temperature may also be used as an enhancer of the gelation process. In addition to this, manipulation of intermediate silk solution pH by methods including but not limited to direct titration and gas exchange may be used to enhance the gelation process. Introduction of select ionic species including calcium and potassium in particular may also be used to accelerate gelation rate.

The amount of time required for dissolved silk solutions to gel may vary from seconds to hours or days, depending on the ratio of silk solution volume and enhancer solution volume, dissolved silk fibroin concentration, enhancer solution concentration, enhancer type, am phiphilic peptide concentration, temperature, salt concentrations (e.g. CaCl₂, NaCl, and KCl), and pH as well as the initial state of aggregation and organization found in the silk solution. The amphiphilic peptide may be mixed into the dissolved silk solution in a variety of ways, for example water-dissolved amphiphilic peptide can be added to a dissolved silk solution to form a gel; an amphiphilic peptide can be added to water, blended with an alcohol, then added to a dissolved silk solution; or an amphiphilic peptide can be added to a silk fibroin biomaterial. The molar ratio of amphiphilic peptide:silk fibroin can range from 100 to 0.01, the dissolved silk solution concentration can be from about 1% to about 20%.

In aspects of this embodiment, an enhancer solution is added to a depolymerized silk fibroin (dissolved silk fibroin) solution, the depolymerized silk fibroin solution having a concentration of depolymerized silk fibroin of, e.g., about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 12% (w/v), about 15% (w/v), about 18% (w/v), about 20% (w/v), about 25% (w/v), or about 30% (w/v). In other aspects of this embodiment, an enhancer solution is added to a depolymerized silk fibroin (dissolved silk fibroin) solution, the depolymerized silk fibroin solution having a concentration of depolymerized silk fibroin of, e.g., at least 1% (w/v), at least 2% (w/v), at least 3% (w/v), at least 4% (w/v), at least 5% (w/v), at least 6% (w/v), at least 7% (w/v), at least 8% (w/v), at least 9% (w/v), at least 10% (w/v), at least 12% (w/v), at least 15% (w/v), at least 18% (w/v), at least 20% (w/v), at least 25% (w/v), or at least 30% (w/v). In yet other aspects of this embodiment, an enhancer solution is added to a depolymerized silk fibroin (dissolved silk fibroin) solution, the depolymerized silk fibroin solution having a concentration of depolymerized silk fibroin of, e.g., about 1% (w/v) to about 5% (w/v), about 1% (w/v) to about 10% (w/v), about 1% (w/v) to about 15% (w/v), about 1% (w/v) to about 20% (w/v), about 1% (w/v) to about 25% (w/v), about 1% (w/v) to about 30% (w/v), about 5% (w/v) to about 10% (w/v), about 5% (w/v) to about 15% (w/v), about 5% (w/v) to about 20% (w/v), about 5% (w/v) to about 25% (w/v), about 5% (w/v) to about 30% (w/v), about 10% (w/v) to about 15% (w/v), about 10% (w/v) to about 20% (w/v), about 10% (w/v) to about 25% (w/v), or about 10% (w/v) to about 30% (w/v).

Silk fibroin microcapsules can be produced by preparing a solution comprising a concentrated substantially sericin-depleted silk fibroin, mixing the silk fibroin solution with a compound disclosed herein and/or other components disclosed herein, and with a lipid composition, lyophilizing the mixture, combining the lyophilized mixture with a dehydration medium for a sufficient period of time to at least partially dehydrate the silk fibroin solution and induce β-sheet structures in the silk fibroin, and removing at least a portion of the lipids to produce a compound disclosed herein and/or other components disclosed herein encapsulated in a silk fibroin microcapsule. Similarly, silk fibroin microspheres can be produced by preparing a solution comprising a concentrated substantially sericin-depleted silk fibroin, mixing the silk fibroin solution with a compound disclosed herein and/or other components disclosed herein, and with a lipid composition, lyophilizing the mixture, combining the lyophilized mixture with a dehydration medium for a sufficient period of time to at least partially dehydrate the silk fibroin solution and induce β-sheet structures in the silk fibroin, and removing at least a portion of the lipids to form silk fibroin microspheres comprising a compound disclosed herein and/or other components disclosed herein. Such methods are described in, e.g., Kaplin, et al., Silk Microspheres for Encapsulation and Controlled Release, U.S. Patent Application 2010/0028451, which is hereby incorporated by reference in its entirety.

Lipid vesicles are used in the process as templates to assist in modeling the microcapsules/microspheres into preferred shapes and sizes. The lipid composition may include any lipid or combination of lipids that can form liposomes. Suitable lipids in the lipid composition include, without limitation, 1,2-dioleoyl-sn-glycero-3-phosphocholine (DOPC); 1,2-dioleoyl-sn-glycero-3-phophoethanolamine (DOPE); 1,2-dilauroyl-sn-glycero-3-phosphocholine (DLPC); and 1,2-dimyristoyl-sn-glycero-3-phosphocholine (DMPC). Other lipid compositions known in the art may also be used.

The silk fibroin solution and lipid composition should be mixed in a manner that integrates the silk fibroin and lipids, as well as any other compounds or components. Preferably, the mixing takes place for a sufficient period of time and under conditions so that the various components are significantly integrated. The amount of silk fibroin solution and lipid composition that is mixed is dependent on the dehydrating medium used and the desired structural formation of the microspheres. Typically, 0.1 mL to 2 mL of 8% (w/v) silk solution is used for every 100 mg of lipids. However, these amounts may vary depending on the exact make up of the silk solution and lipid composition. Additionally, depending on the dehydrating medium used, each medium will have a threshold ratio. For instance, when methanol is used as the dehydrating medium, the threshold ratio is 0.2 mL of 8% (w/v) silk solution for every 100 mg of lipids, and when sodium chloride is used as the dehydrating medium, the threshold ratio is 0.5 mL of 8% (w/v) silk solution for every 100 mg of lipids. When the amount of lipids are above the threshold ratio, multilamellar structures predominately form in the microspheres; when the amount of lipids are below the threshold ratio, unilamellar structures predominately form in the microspheres.

Sufficient mixing is sometimes difficult to achieve. In such cases, a freeze-thaw step may be used, which promotes mixing among the lipids, silk fibroin, and compounds, when present. A freeze-thaw step can break larger multilamellar lipid vesicles into smaller, unilamellar structures that have more homogeneous size distributions. It can also be used to facilitate silk self assembly and enhance the encapsulation of the compound disclosed herein in the liposomes. Any freeze-thaw treatment known in the art may by used. See, e.g., Colletier et al., Protein encapsulation in liposomes: efficiency depends on interactions between protein and phospholipid bilayer, BMC Biotechnology 2 (2002) 9-17, hereby incorporated by reference in its entirety, for suitable freeze-thaw techniques. The freeze-thaw may be repeated one or more times to promote further mixing and size homogeneity. Freeze-thawing is not deemed necessary when using certain dehydrating mediums, such as methanol, where the particle-size distribution and integration level achieved through mixing alone is usually adequate.

The lipid components that remain in the microcapsules/microspheres will form as either uni- or multilamellar structures. Compared to multilamellar lipid vesicles, unilamellar vesicles offer higher encapsulation capacity for hydrophilic drugs, more reproducible rates of release, and less lipid content in the microcapsules/microspheres. On the other hand, multilamellar vesicles are suitable for encapsulating both lipophilic and hydrophilic drugs and are more resistant to enzyme digestion, resulting in a longer circulation time in the body. Therefore, unilamellar-structured microcapsules/microspheres are generally preferred when higher drug loading is needed or when hydrophilic drugs are used; multilamellar-structured microcapsules/microspheres are generally preferred when lipophilic drugs are used and in cases when drug loading is not important or when a slower degradation of microcapsules/microspheres is desired. In addition to vesicle structure (uni- or multilamellar), the drug release rate is also governed by lipophilicity of drug molecules, the composition of the encapsulation device, and the lipid composition.

After the silk fibroin solution and the lipid composition have been mixed and optionally freeze-thawed, the mixture is lyophilized. Lyophilization techniques known in the art may be used. Typically, the mixture is lyophilized for three days and stored at temperatures around 4° C.

The lyophilized mixture is then combined with a dehydration medium. The dehydration medium may be any medium that can both dehydrate the silk fibroin solution and induce β-sheet structures in the silk fibroin. Dehydrating the silk fibroin solution extracts water from the silk fibroin and causes the silk to self assemble and form crystalline β-sheet structures. The β-sheet structures are physical crosslinks in the silk fibroin that provide the silk with stability and unique mechanical features in the fibers. The physical crosslinks also promote the entrapment of therapeutic agents, when present, in the silk fibroin. β-sheet structures in the silk fibroin may also be induced by changes in salt concentration and shear forces.

Microcapsules/microspheres form upon crosslinking of the silk fibroin. Preferably, the weight percentage of microcapsules/microspheres in the total silk is at least about 50%. The amount of microcapsules/microspheres in the silk is dependent on various factors, such as the dehydration agent used, the type of silk fibroin, the amount of time the silk is exposed to the dehydration agent, etc.

The dehydration medium should at least partially dehydrate the silk fibroin. Preferably, the silk fibroin is sufficiently dehydrated so that significant amounts (e.g. 50% or more) of β-sheet structures form in the silk. The amount of dehydration time necessary to induce β-sheet formation is readily determinable by one skilled in the art and will depend, in part, on the dehydration medium used. Because high crystallinity can significantly retard the release of encapsulated therapeutic agents, such as proteins, inducing large amounts of β-sheet formation is preferable when forming microcapsules/microspheres designed for control release.

Any known dehydration medium that does not destroy or otherwise damage the silk fibroin may be used as the dehydration medium. Polar alcohols, such as methanol and ethanol, are particularly effective at inducing dehydration of the silk. Other polar solvents, like ketones, such as acetone, are also effective. Solvents and alcohols with lower polarity, like trihalomethanes such as chloroform and alcohols such as propanol, may also be used, but are not as effective at stabilizing the silk structure. Additionally, many salts, such as sodium chloride and potassium chloride, can dehydrate the silk fibroin as well change the salt concentration, both of which induce β-sheet formation. Other suitable dehydration mediums include polyethylene glycol solutions, desiccants, and dry gas. Preferably, the dehydration medium is a polar solvent, such as methanol, ethanol, and acetone, or a salt, such as sodium chloride or potassium chloride. Methanol and solutions of sodium chloride are particularly preferred.

The lyophilized mixture and dehydration medium may be combined through any method known in the art. Preferably, the dehydration medium is in a solution and the lyophilized mixture is combined with it by adding the lyophilized mixture to the solution. Combining the two components in this manner will typically form a suspension of the lyophilized mixture in the dehydration medium solution. When the lyophilized mixture is suspended in the solution, it allows for easier removal of the lipids.

At least some of the lipids should be removed after the lyophilized mixture has been combined with the dehydration medium. The lipids may be removed through any technique known in the art. Centrifugation may be used when the lyophilized mixture is suspended in a solution containing the dehydration medium, however, other removal or extraction techniques may be better suited to remove the lipids depending on the dehydration medium utilized. When silk fibroin microcapsules/microspheres are prepared with a process that utilizes lipid components, a portion of the lipid components is typically present in the silk fibroin microcapsules/microspheres, even when all of the removable lipid components have been removed. Depending on the process used to incorporate and/or remover the lipids, lipid components will typically be present in the microcapsules/microspheres from about 1 to about 25%, by weight. Preferably, the microcapsules/microspheres contain less than about 20% lipids by weight, more preferably less than about 5% lipids by weight. It is believed that the lipids, when present in relatively small amounts, assist in controlling the release of the therapeutic agent from the microcapsules/microspheres. When the microcapsules/microspheres contain too high a percentage of lipids, the structure and physical parameters of the silk fibroin microcapsules/microspheres can be compromised, resulting in less effective microcapsules/microspheres or microcapsules/microspheres with insufficient structural integrity.

Certain dehydration mediums can function to remove the lipids. For instance, a high concentration of methanol or sodium chloride enables each medium to function as both a dehydration medium and lipid remover. Additional removal steps, such as centrifugation, are nonetheless still preferred even when using methanol or sodium chloride. Other dehydration mediums, such as desiccants or dry gas, function little if at all as a lipid remover. These type of dehydration mediums, therefore, may have to be combined with a more rigorous lipid extraction or removal step, or multiple extraction/removal steps.

It is preferable to remove all or substantially all of the removable lipids. Depending on the removal techniques and dehydration medium used, complete lipid removal may not be possible. For instance, when using methanol as the dehydration medium, about 99% of the lipids are able to be removed; when using sodium chloride as the dehydration medium, about 83% of the lipids are able to be removed. In these cases, all or substantially all of the removable lipids are considered to have been removed because further removal techniques would not lead to any substantial amount of additional lipids being removed.

While it is preferable to remove most of the lipids, it is also believed that the lipid components, when present in a relatively small amount, can be beneficial. In particular, it is believed that the lipid component can assist in controlling the release of the therapeutic agent from the microcapsules/microspheres. Therefore, according to an embodiment of the invention, it is preferable to have a microcapsules/microspheres composition where about 15 to about 20% of the total lipids remain in the silk fibroin microcapsules/microspheres. It is also preferable to have a microsphere composition where less than about 5% of the total lipids remain in the silk fibroin microcapsules/microspheres. More preferably, less than about 2% of the total lipids remain in the microcapsules/microspheres.

After the desired amount of lipids have been removed, the composition is typically in a dehydrated pellet form. The composition may be hydrated by suspending or resuspending the microcapsules/microspheres composition in water or a buffer solution. Suspending the microspheres in water or a buffer is often done before the microcapsules/microspheres composition is used in a commercially viable manner. For instance, if the silk fibroin microcapsules/microspheres are used in a formulation suitable for administration, the formulation will typically contain hydrated microcapsules/microspheres.

Silk fibroin sheets or films can be produced by preparing a solution comprising a concentrated substantially sericin-depleted silk fibroin, mixing a compound having the structure of formula I as disclosed herein and/or other components disclosed herein, casting the solution onto a flat surface or mold, and drying the silk mixture. Alternatively, the casted solution can be contacted with an alcohol/water solution to crystallize the silk solution. The film may then be contacted with water or water vapor, in the absence of alcohol, to make the film playable, and then drawn or stretched mono-axially or biaxially. The stretching of a silk film induces molecular alignment of the film and thereby improving its mechanical properties. Such methods are described in, e.g., Kaplin, et al., Silk Boimaterals and Methods of Use Thereof, U.S. Pat. No. 7,674,882; Kaplin, et al., Concentrated Aqueous Silk Fibroin Solution and Use Thereof, U.S. Pat. No. 7,635,755; Kaplin, et al., Methods for Stepwise Deposition of Silk Fibroin Coatings, U.S. Patent Publication 2009/0202614; Kaplin, et al., Silk Fibroin Materials and Use Thereof, U.S. Patent Publication 2006/0273279; Lu, et al., Modified Silk Films Containing Glycerol, International Patent Publication WO 2004/001103; and Kaplin, et al., Silk Biomaterials and Methods of Use Thereof, International Patent Publication WO 2010/042798, each of which is herein incorporated by reference in its entirety. Preferably, the resulting silk fibroin film is from about 60 μm to about 240 μm thick, however, thicker samples can easily be formed by using larger volumes of concentrated aqueous silk fibroin solution or by depositing multiple layers. To make a multi-layer film, preparing a solution comprising a concentrated substantially sericin-depleted silk fibroin onto a flat surface or mold, and drying the silk mixture, such as, e.g., by exposing the layer to a flow of dehydrating gas like air, CO₂ or N₂. The dehydrated first layer is then contacted with a concentrated silk fibroin solution such that the solution forms a second layer upon the dehydrated first layer and drying the silk mixture, such as, e.g., by exposing the layer to a flow of dehydrating gas. This process is repeated until the desired numbers of layers are deposited upon the substrate resulting in a layered film. One, a plurality, or all layers comprising the multi-layered film can include a compound disclosed herein, an antibiotic, and/or other components disclosed herein by adding the compound, antibiotic or other component to the concentrated silk fibroin solution before casting and/or contacting the solution to the surface or dehydrated layer.

In one embodiment, the film comprises a blend of silk fibroin and another biocompatible polymer. For example, a film may comprise from about 50 to about 99.99 parts by volume aqueous silk fibroin solution and from about 0.01 to about 50 parts by volume of polyethylene oxide (PEO). Useful biocompatible polymers include, without limitation, polyethylene oxide (PEO) (U.S. Pat. No. 6,302,848), polyethylene glycol (PEG) (U.S. Pat. No. 6,395,734), collagen (U.S. Pat. No. 6,127,143), fibronectin (U.S. Pat. No. 5,263,992), keratin (U.S. Pat. No. 6,379,690), polyaspartic acid (U.S. Pat. No. 5,015,476), polylysine (U.S. Pat. No. 4,806,355), alginate (U.S. Pat. No. 6,372,244), chitosan (U.S. Pat. No. 6,310,188), chitin (U.S. Pat. No. 5,093,489), hyaluronic acid (U.S. Pat. No. 387,413), pectin (U.S. Pat. No. 6,325,810), polycaprolactone (U.S. Pat. No. 6,337,198), polylactic acid (U.S. Pat. No. 6,267,776), polyglycolic acid (U.S. Pat. No. 5,576,881), polyhydroxyalkanoates (U.S. Pat. No. 6,245,537), dextrans (U.S. Pat. No. 5,902,800), and polyanhydrides (U.S. Pat. No. 5,270,419). Two or more biocompatible polymers can be used.

Silk fibroin porous materials may be made using methods known in the art. For example, a silk fibroin porous material can be made using a gas foaming process where water is the solvent or nitrogen or other gas is the blowing agent. Alternatively, a porous material can be made using freeze-drying process, a solvent casting/particulate leaching process. Such methods are described in, e.g., Kaplin, et al., Concentrated Aqueous Silk Fibroin Solution and Use Thereof, U.S. Pat. No. 7,635,755; Ratner and Marshall, Novel Porous Materials, U.S. Patent Publication 2008/0075752; Ma and Chen, Porous Materials having Multi-Sized Geometries, U.S. Patent Publication 2007/0036844; Kaplan, et al., Electrospun Pharmaceutical Compositions, U.S. Patent Publication 2006/0273279; Ma, Reverse Fabrication of Porous Materials, U.S. Patent Publication 2002/0005600; Liu, et al., Porous Materials, Methods of Making and Uses, U.S. Patent Application 61/333,613, filed on May 11, 2010; and Liu, et al., Porogen Compositions, Methods of Making and Uses, U.S. Patent Application 61/333,120, filed on May 10, 2010, each of which is hereby incorporated by reference in its entirety. For example, a porous material can be made by preparing a solution comprising a concentrated substantially sericin-depleted silk fibroin, adding a compound disclosed herein and/or other components disclosed herein, mixing the solution with water-soluble non-toxic porogens that are insoluble in organic solvents, placing the mixture into a mold or onto flat surface, contacting the mixture with an effective amount of an agent to induce β-sheet structure and insolubility in aqueous solution and leaching the material with a solvent that can dissolve the porogens but not the material, and drying the resulting porous material. The resultant porous material can then be dried and used, e.g., as a cell scaffold in biomedical application.

The preferred silk fibroin concentration of the concentrated aqueous silk fibroin solution is from about 5% to about 35%. The preferred diameter of the porogen is between about 50 μm and about 1000 μm. The pore size can be controlled by adjusting the concentration of silk fibroin and the particle size of a porogen. Examples of water-soluble porogens useful according to the disclosed methods include, without limitation, monovalent or divalent salts, alkali metals, alkali earth metal halides, phosphates, and sulfates, sugar crystals, polysaccharides and proteins.

The silk fibroin solutions disclosed herein are amorphous in structure and water-soluble. As such, prior to submersion into aqueous solutions the silk fibroin-coated porogens are first exposed to a β-sheet structure inducing agent, such as alcohol or salt, to induce the phase transition or conformational change of the amorphous silk fibroin to β-sheet structure that is insoluble in the solution. Different β-sheet structure inducing agents can be used to generate scaffolds with different properties.

The β-sheet structure induced silk fibroin is immersed in water or other solvent in which the particles, or porogens are soluble and polymer is insoluble, to remove the particles, resulting in a porous three-dimensional structure, referred herein to as a “silk fibroin scaffold.” Useful solvents useful include, without limitation, water, hexa-fluoro-iso-propanyl (HFIP), N-methyl morpholine N-oxide and calcium nitrate-methanol. The solvent is then removed using, e.g., sublimation or evaporation.

A porous silk fibroin biomaterial disclosed herein comprising a three-dimensional silk fibroin body having interconnected pores therein. The pores having a diameter of from about 50 μm to about 1000 μm. The pore density is from about 20 mg/mL to about 200 mg/mL, preferably from about 40 mg/mL to about 150 mg/mL. Porosity ranges from about 50% to about 99.5%, preferably about 80 to about 95%. Most preferably the porosity is above 85%. The density or porosity can be adjusted depending on the use of material. Preferably, the material has a compressive modulus of at least 100 kPa. More preferably, the material has a compressive modulus of at least 150 kPa. Even more preferably, the material has a compressive modulus of 200 kPa. Most preferably, the material has a compressive modulus of 250 kPa.

Silk fibroin textured or micropatterned materials may be made using methods known in the art. Such methods are described in, e.g., Chun, et al., Methods of Making Micropatterned Foams, U.S. Pat. No. 6,423,252, which is hereby incorporated by reference in its entirety. The method comprises contacting a solution comprising a concentrated substantially sericin-depleted silk fibroin with a surface of a mold, the mold comprising on at least one surface thereof a three-dimensional negative configuration of a predetermined micropattern to be disposed on and integral with at least one surface of the material, lyophilizing the solution while in contact with the micropatterned surface of the mold, thereby providing a lyophilized, micropatterned material, and removing the lyophilized, micropatterned material from the mold. Textured materials prepared by this method comprise a predetermined and designed micropattern on at least one surface, which pattern is effective to facilitate tissue repair, ingrowth or regeneration.

Silk fibroin fibers or filaments may be made using methods known in the art. For example, fibers may be produced using, e.g., wet spinning or electrospinning. Alternatively, as the concentrated solution becomes a taffy-like consistency, a fiber can be pulled directly from the solution. Electrospinning can be performed by any means known in the art. See, e.g., Zarkoob, et al., Synthetically Spun Silk Nanofibers and a Process for Making the Same, U.S. Pat. No. 6,110,590; Altman, et al., Sericin Extracted Silkworm Fibroin Fibers, U.S. Patent Publication 2010/0209405, each of which is hereby incorporated by reference in its entirety.

For example, a steel capillary tube with a 1.0 mm internal diameter tip may be mounted on an adjustable, electrically insulated stand. The capillary tube is maintained at a high electric potential and mounted in the parallel plate geometry and is connected to a syringe pump filled with silk solution. A constant volume flow rate is maintained using the syringe pump, set to keep the solution at the tip of the tube without dripping. The electric potential, solution flow rate, and the distance between the capillary tip and the collection screen are adjusted so that a stable jet is obtained. Dry or wet fibers are collected by varying the distance between the capillary tip and the collection screen. A collection screen suitable for collecting silk fibers can be a wire mesh, a polymeric mesh, or a water bath. Alternatively and preferably, the collection screen is an aluminum foil. The aluminum foil can be coated with non-stick fluid, such as, e.g., a fluorocarbon polymer like polytetrafluoroethylene, to make peeling off the silk fibers easier. One skilled in the art will be able to readily select other means of collecting the fiber solution as it travels through the electric field. The electric potential difference between the capillary tip and the aluminum foil counter electrode is, preferably, gradually increased to about 12 kV, however, one skilled in the art should be able to adjust the electric potential to achieve suitable jet stream.

The present specification additionally provides a non-woven network of fibers comprising silk fibroin biomaterials disclosed herein. The fiber may also be formed into yarns and fabrics including for example, woven or weaved fabrics. See, e.g. Kaplin, et al., Silk Boimaterals and Methods of Use Thereof, U.S. Pat. No. 7,674,882; Kaplin, et al., Concentrated Aqueous Silk Fibroin Solution and Use Thereof, U.S. Pat. No. 7,635,755; Kaplin, et al., Silk Fibroin Materials and Use Thereof, U.S. Patent Publication 2006/0273279; and Kaplin, et al., Silk Biomaterials and Methods of Use Thereof, International Patent Publication WO 2004/001103, each of which is herein incorporated by reference in its entirety.

The silk fibroin biomaterials disclosed herein may also be coated onto various shaped substrates, including articles, such as, e.g., biomedical devices like implants, pacemakers, and stents); and silk or other fibers, and fragments thereof. For example, a substrate can be coated with a silk fibroin by preparing a solution comprising a concentrated substantially sericin-depleted silk fibroin, a compound having the structure of formula I as disclosed herein and/or other components as disclosed herein, contacting the substrate with the silk fibroin solution such that the solution forms a layer upon the substrate, and dehydrating the layer. Dehydration can be achieved by exposing the silk fibroin layer to a flow of a dehydrating gas. To form multiple layers of silk fibroin on a substrate, the substrate with the first dehydrated layer of silk fibroin is contacted a silk fibroin solution (with or without a compound as disclosed herein and/or other components as disclosed herein) such that the solution forms a second layer upon the dehydrated first layer, and dehydrating the second layer. These steps can be repeated until the desired number of dehydrated silk fibroin layers are deposited upon the substrate resulting in a silk fibroin layered coating on the substrate. Such methods are described in, e.g., Kaplin, et al., Silk Boimaterals and Methods of Use Thereof, U.S. Pat. No. 7,674,882; Kaplin, et al., Concentrated Aqueous Silk Fibroin Solution and Use Thereof, U.S. Pat. No. 7,635,755; Kaplin, et al., Silk Fibroin Materials and Use Thereof, U.S. Patent Publication 2006/0273279; and Kaplin, et al., Silk Biomaterials and Methods of Use Thereof, International Patent Publication WO 2004/001103, each of which is herein incorporated by reference in its entirety. A silk fibroin layered coated-substrate may also comprise a porous or textured or micropatterned surface as described herein.

As used herein, the term “contacting” when referring to “contacting a substrate” or “contacting a dehydrated silk fibroin layer” refers to any means for applying a silk solution to a substrate or dehydrated silk fibroin layer. For example, the aqueous silk fibroin solution can be poured, or sprayed, onto the substrate or dehydrated layer either with or without the aid of a casting structure. Alternatively, the substrate, or substrate comprising a dehydrated fibroin layer, can be dipped into the silk fibroin solution. Automated means are also contemplated. The silk fibroin solution may be coated onto any substrate of any size or shape. The substrate can be of a natural or synthetic nature. For example, the substrate can be made of an elastomer like silicone, a plastic, wood, glass, leather, cloth, synthetic fibers or any metal or alloy.

The deposited aqueous silk fibroin layers are dehydrated using a stream or gentle flow of dehydrating gas. Any gas with dehydrating properties can be used to dehydrate the aqueous silk fibroin layers with the proviso that the gas induces a β-sheet structure of silk fibroin. Non-limiting examples of suitable dehydrating gases, include, air, CO₂, and N₂. In addition, means for dehydrating gases are known to those skilled in the art. The layers can be dehydrated to various degrees by changing the amount of time each layer is exposed to the stream of gas. As used herein, the term “dehydrating” refers to the removal of any amount of water, for example, 5-15%, 15-35%, 35-50%, 50%-75%, 75-90%, or 90%-100% removal of water.

The degradation rate of a silk fibroin layer can be controlled by adjusting the degree that each layer is dehydrated and/or by adjusting the thickness of each layer. The thickness of each deposited layer can be controlled by adjusting a variety of parameters, including adjusting the concentration of salt, the concentration of fibroin, and the pH of the aqueous silk fibroin solution used to form the layer and the rinsing medium (water and methanol). The level of dryness/dehydration can be adjusted by adjusting the amount of time that the layer is exposed to the dehydrating gas. The concentration of salt is increased to favor deposition of silk fibroin onto the substrate. Salt concentration can be increased by addition of any salt to the aqueous silk fibroin solution including, but not limited to, monovalent and divalent salts such as, e.g., NaCl, KCl and CaCl₂. When fibroin is deposited on a hydrophobic substrate, increasing the salt concentration increases the amount of fibroin deposited on the substrate resulting in a more compact structure of fibroin chains. The thickness of each deposited layer can also be controlled by adjusting the concentration of fibroin in the silk fibroin solution used to form the layer. The more concentrated the fibroin in the aqueous silk fibroin solution is, the more fibroin that is deposited on the substrate and a more compact structure is formed. Adjusting the pH of the aqueous silk fibroin solution also affects the amount of fibroin deposited on the substrate. When the substrate is a negatively charged substrate, lowering the pH of the silk fibroin solution favors deposition of the silk fibroin onto the substrate. When the substrate is a positively charged substrate, increasing the pH of the silk fibroin solution favors deposition of the silk fibroin onto the substrate. At a low pH (e.g. 2.0) the silk fibroin chains have a net positive charge, which favors deposition on a negative substrate. In contrast, at a high pH (e.g. 12.5) the silk fibroin chains have a net negative charge, and thus, deposition on a negatively charged substrate is not favored.

In aspects of this process, silk fibroin is used to coat an implantable medical device that undergoes flexion or expansion in the course of its implantation or use in vivo. The words “flexion” and “expansion” as used herein with regard to implantable devices will refer to a device, or portion thereof, that is bent (e.g., by at least 45 degrees or more) and/or expanded (e.g., to more than twice its initial dimension), either in the course of its placement, or thereafter in the course of its use in vivo. Any biomedical device can be coated using the disclosed methods.

The suitability of the fibroin coating composition for use on a particular material, and in turn, the suitability of the coated composition can be evaluated by those skilled in the art, given the present description. For example, a substrate used for coating can be a catheter. Examples of suitable catheters include urinary catheters, which would benefit from the incorporation of antimicrobial agents (e.g., antibiotics such as vancomycin or norfloxacin) into a surface coating, and intravenous catheters which would benefit from antimicrobial agents and or from antithrombotic agents (e.g., heparin, hirudin, coumadin). Such catheters are typically fabricated from materials like silicone, polyurethane, latex and polyvinylchloride.

A substrate used for coating can also be a stent, e.g., either self-expanding stents (such as the Wallstent variety), or balloon-expandable stents (as are available in a variety of styles, for instance, Gianturco-Roubin, Palmaz-Shatz, Wiktor, Strecker, ACS Multi-Link, Cordis, AVE Micro Stent), which are typically prepared from materials such as stainless steel or tantalum.

A substrate used for coating can also be an implantable device. An implantable device is synonymous with “medical device”, “biomedical device”, “implantable medical device” or “implantable biomedical device” and includes, without limitation, pacemakers, dura matter substitutes, implantable cardiac defibrillators, tissue expanders, and tissue implants used for prosthetic, reconstructive, or aesthetic purposes, like breast implants, muscle implants or implants that reduce or prevent scarring. Examples of implantable devices that can be coated with silk fibroin as disclosed herein are described in, e.g., Schuessler, Rotational Molding System for Medical Articles, U.S. Pat. No. 7,628,604; Smith, Mastopexy Stabilization Apparatus and Method, U.S. Pat. No. 7,081,135; Knisley, Inflatable Prosthetic Device, U.S. Pat. No. 6,936,068; Falcon, Reinforced Radius Mammary Prostheses and Soft Tissue Expanders, U.S. Pat. No. 6,605,116; Schuessler, Rotational Molding of Medical Articles, U.S. Pat. No. 6,602,452; Murphy, Seamless Breast Prosthesis, U.S. Pat. No. 6,074,421; Knowlton, Segmental Breast Expander For Use in Breast Reconstruction, U.S. Pat. No. 6,071,309; VanBeek, Mechanical Tissue Expander, U.S. Pat. No. 5,882,353; Hunter, Soft Tissue Implants and Anti-Scarring Agents, Schuessler, Self-Sealing Shell For Inflatable Prostheses, U.S. Patent Publication 2010/0049317; U.S. 2009/0214652; Schraga, Medical Implant Containing Detection Enhancing Agent and Method For Detecting Content Leakage, U.S. Patent Publication 2009/0157180; Schuessler, All-Barrier Elastomeric Gel-Filled Breast Prosthesis, U.S. Patent Publication 2009/0030515; Connell, Differential Tissue Expander Implant, U.S. Patent Publication 2007/0233273; and Hunter, Medical implants and Anti-Scarring Agents, U.S. Patent Publication 2006/0147492; Van Epps, Soft Filled Prosthesis Shell with Discrete Fixation Surfaces, International Patent Publication WO/2010/019761; Schuessler, Self Sealing Shell for Inflatable Prosthesis, International Patent Publication WO/2010/022130; Yacoub, Prosthesis Implant Shell, International Application PCT/US09/61045, each of which is hereby incorporated by reference in its entirety.

The silk fibroin biomaterials disclosed herein can be further modified after fabrication. For example, the scaffolds can be coated with additives, such as bioactive substances that function as receptors or chemoattractors for a desired population of cells. The coating can be applied through absorption or chemical bonding. See, e.g. Kaplin, et al., Silk Boimaterals and Methods of Use Thereof, U.S. Pat. No. 7,674,882; Kaplin, et al., Concentrated Aqueous Silk Fibroin Solution and Use Thereof, U.S. Pat. No. 7,635,755; Kaplin, et al., Silk Fibroin Materials and Use Thereof, U.S. Patent Publication 2006/0273279; and Kaplin, et al., Silk Biomaterials and Methods of Use Thereof, International Patent Publication WO 2004/001103, each of which is herein incorporated by reference in its entirety.

The silk fibroin biomaterials disclosed herein, such as, e.g., hydrogels, films, porous materials, fibers, meshes, mats, microcapsules or microspheres may be used in a variety of medical applications such as a drug delivery platform, administering, e.g., a small molecule, nucleic acid, or a biologic, including controlled release systems, wound closure systems, including vascular wound repair devices, hemostatic dressings, patches and glues, sutures, and in tissue engineering applications, such as, e.g., scaffolds for tissue regeneration, ligament prosthetic devices and in products for long-term or bio-degradable implantation into the human body. Films may also be used for a wide range of materials science and engineering needs, such as controlled drug release systems, coatings, composites or as stand alone materials. The incorporated platform then administers the compound to the wound by simple diffusion from the dressing to promote wound healing and reduce scar formation.

For example, a drug delivery platform disclosed herein can be made into a filaments or fibers to produce sutures used to close an open wound. Similarly, a drug delivery platform disclosed herein may be processed into a staple or other closure device used to close an open wound. Administration of the compound occurs by diffusion to promote wound healing and reduce scar formation. As used herein, the term “wound” refers to a damaged tissue like a skin breakage, such as, e.g., a laceration, cut, tear, puncture, abrasion, ulceration, or other open wound of the skin; a burn, such as, e.g., a thermal, chemical or electrical burn; a bone fracture or break; a muscle, ligament or tendon tear or sprain; a damaged joint, or any other tissue damage that requires healing of the tissue as described herein. Methods of treating wounds using the drug delivery platforms disclosed herein are described in, e.g., Jiang, et al., Compositions and Methods for Skin Repair, U.S. Patent Application 61/374,439, which is hereby incorporated by reference in its entirety.

As another example, a drug delivery platform comprising disclosed herein can be made into a wound dressing, such as, e.g., a gauze pad or mat, that is applied over a wound to close, cover, seal, or otherwise protect the wound from infection and the environment. Such a dressing may also be incorporated into a bandage to facilitate and/or maintain proper placement and/or adherence of the dressing over the wound. Administration of the compound would occur by diffusion thereby promoting wound healing and reducing scar formation. Likewise, a wound dressing including such a platform can be applied to a bone fracture or break, a muscle, ligament or tendon tear or sprain, or a damaged joint to promote wound healing and/or structural support. In applications involving wound healing, a drug delivery platform disclosed herein can be processed to further comprise an antibiotic or anesthetic. A wound dressing delivery platform disclosed herein can be processed to further comprise an antibiotic or anesthetic.

As another example, a drug delivery platform comprising disclosed herein can be made into a single or multi-layered sheet or film that is applied to a wound, such as a skin wound involving the epidermis, dermis and/or hypodermis. Such a sheet or film may be transparent or translucent to facilitate healthcare provider monitoring or opaque to mask the wound. For instance, a silk fibroin film comprising a compound disclosed herein can be applied over and/or under a surgical incision in the epidermis, dermis and/or hypodermis after a breast surgery like a breast augmentation, breast reconstruction, and/or breast lift; an abdominal surgery, like an abdominoplasty; a facial surgery like a face lift; or other body contouring procedure. Administration of the compound would occur by diffusion thereby promoting wound healing and reducing scar formation. A sheet or film drug delivery platform disclosed herein can be processed to further comprise an antibiotic or anesthetic. Such additional components can be within the same layer as the compound disclosed herein, or on separate layers in a multi-layered sheet or film. Additionally, multi-layered sheets or films can be designed in such a manner as to provide different release rates of a compound, antibiotic, anesthetic, or other components contained within the same or different layers. A sheet or film drug delivery platform disclosed herein may also comprise an adhesive and thus further serve as a bandage. A sheet or film drug delivery platform as disclosed herein also facilitates oxygen diffusion because of the thin nature of the format.

Additionally, a drug delivery platform comprising disclosed herein can be made into a transparent or translucent sheet or film that is applied over the cornea of an eye to treat corneal wound, corneal haze or a similar opacity condition. For instance, a transparent or translucent sheet or film is the form of a contact lens could be applied over the cornea after a laser refractive treatment for correcting myopia, hyperopia, and/or astigmatism. Administration of the compound would occur by diffusion thereby promoting wound healing and reducing scar formation. Methods of treating corneal haze and similar conditions using the drug delivery platforms disclosed herein are described in, e.g., Jiang, et al., Compositions and Methods for Treating Corneal Haze, U.S. Patent Application 61/369,232, which is hereby incorporated by reference in its entirety.

As another example, a drug delivery platform disclosed herein may be pulverized into particles and mixed with water or a saline solution to form an injectable or topical substance like a solution, oil, lotion, gel, ointment, cream, slurry, salve, or paste. A silk fibroin biomaterial may be milled to a particle size from about 0.1 μm to about 1000 μm in diameter, such as 15 μm to 30 μm. Saline is then added as a carrier phase by first determining the bulk volume of the biomaterial, then vigorously pulverizing the biomaterial into particles while incorporating an appropriate volume of saline to achieve a desired carrier to biomaterial particle ratio. For example, biomaterial milling may be accomplished by means of a forced sieving of bulk platform through a series of stainless steel cloth sieves of decreasing pore sizes. In another example, a drug delivery platform disclosed herein may be loaded into a syringe and pulverized with a spatula to a fine paste with saline. Such a drug delivery platform may be formulated into a lotion, gel, cream, paste, or ointment and administered by injection or applied topically. In another example, a hydrogel may be comminuted into particles in a range of 0.5 to 2 μm diameter using a planetary ball mill and appropriate grinding media.

For example, a drug delivery platform disclosed herein can be formulated into a lotion and topically applied to a laceration, burn or other wound to promote healing. As another example, a drug delivery platform disclosed herein can be formulated into a cream or ointment and topically applied to a surgical incision from a surgery like a face lift to promote healing and reduce scar formation. As still another example, a drug delivery platform disclosed herein can be formulated into a cream and topically applied to treat an acute or chronic skin condition like acne or psoriasis to promote wound healing of the skin and reduce scar formation. Similarly, a drug delivery platform disclosed herein can be processed into a skin care product such as, e.g., a shaving cream or lotion, a dry skin cream or lotion that is applied to the skin to promote wound healing and reduce scar formation. Methods of treating wounds using the drug delivery platforms disclosed herein are described in, e.g., Jiang, et al., Compositions and Methods for Skin Repair, U.S. Provisional Patent Application 61/374,439, which is hereby incorporated by reference in its entirety.

Additionally, a drug delivery platform disclosed herein can be formulated into a liquid adhesive that is applied over a wound to close, cover, seal, or otherwise protect the wound from infection and the environment. Administration of the compound would occur by diffusion thereby promoting wound healing and reducing scar formation. A liquid adhesive delivery platform disclosed herein can be processed to further comprise an antibiotic or anesthetic.

As yet another example, a drug delivery platform disclosed herein can be made into gel or paste that is injected into an intraocular space to treat an eye condition like glaucoma. Methods of treating eye conditions using the drug delivery platforms disclosed herein are described in, e.g., Donde, et el., 10,10-Dialkyl Prostanoic Acid Derivatives as Agents for Lowering Intraocular Pressure, U.S. Pat. No. 6,875,787; and Donde, et el., 10,10-Dialkyl Prostanoic Acid Derivatives as Agents for Lowering Intraocular Pressure, U.S. Patent Publication 2004/0235958, each of which is hereby incorporated by reference in its entirety.

Similarly, a drug delivery platform disclosed herein can be made into a slurry, paste or other format comprising pulverized hydrogel particles useful as a dermal filler for treating a soft tissue condition, such as, e.g., a soft tissue imperfection, defect, disease, and/or disorder. Non-limiting examples of a soft tissue condition include breast augmentation, reconstruction, defect, or imperfection such as, e.g., micromastia, thoracic hypoplasia, Poland's syndrome, defects due to implant complications like capsular contraction and/or rupture; facial augmentation, reconstruction, defect, or imperfection, such as, e.g., Parry-Romberg syndrome, lupus erythematosus profundus, dermal divots, sunken checks, thin lips, retro-orbital defects, facial fold and/or wrinkles like glabellar lines, nasolabial lines, perioral lines, and/or marionette lines, and/or other contour deformities or imperfections of the face; other soft tissue augmentation, reconstruction, defect, or imperfection, such as, e.g., buttocks, calves, genitals, and/or plantar fat pad and/or other contour deformities or imperfections of a body part, region or area; urinary incontinence, fecal incontinence, other forms of incontinence; and gastroesophageal reflux disease (GERD). For example, a silk fibroin biomaterial comprising a disclosed compound can be made into a hydrogel and milled into particles. This drug delivery platform is then mixed with adipose tissue. The resulting composition is then administered into a soft tissue region to reduce a cosmetic or clinical symptom, and/or a physiological indicator associated with the soft tissue condition. Methods of treating soft tissue conditions using the drug delivery platforms disclosed herein are described in, e.g., Van Epps, et al., Compositions and Improved Soft Tissue Replacement Methods, U.S. Patent Application 61/375,144, which is hereby incorporated by reference in its entirety.

Silk fibroin biomaterial comprising a disclosed compound can be made into a porous material like a sponge. Silk fibroin porous materials comprising the compounds disclosed herein can also be used to promote survival of grafted bone chips or osteoblasts to treat large bone defects or bone non-union.

Additionally, silk fibroin biomaterials can be used for organ repair replacement or regeneration strategies that may benefit from these unique scaffolds, including but are not limited to, spine disc, cranial tissue, dura, nerve tissue, liver, pancreas, kidney, bladder, spleen, cardiac muscle, skeletal muscle, tendons, ligaments and breast tissues. For example, a plate, pin, screw or other structural support can be made from silk fibroin biomaterial which further comprises a compound having a structure of formula I disclosed herein. Such a structural support material can be applied to a bone fracture site to accelerate fracture healing. Furthermore, such structural support material can be made biodegradable, thereby eliminating the need of a second surgery to remove the support after bone healing.

Aspects of the present specification provide, in part, a drug delivery platform comprising a carrier. As sued herein, the term “carrier” is synonymous with “carrier phase” and refers to a material used to increase fluidity of the silk fibroin biomaterial. A carrier is advantageously a physiologically-acceptable carrier and may include one or more conventional excipients useful in pharmaceutical compositions. As used herein, the term “a physiologically-acceptable carrier” refers to a carrier in accord with, or characteristic of, the normal functioning of a living organism. As such, administration of a drug delivery platform comprising a carrier has substantially no long term or permanent detrimental effect when administered to a mammal. The present drug delivery platform includes a carrier where a major of the volume is water or saline. However, other useful carriers include any physiologically tolerable material which improves upon extrudability or intrudability of the drug delivery platform through a needle or into a target host environment. Potential carriers could include but are not limited to physiological buffer solutions, serum, other protein solutions, gels composed of polymers including proteins, glycoproteins, proteoglycans, or polysaccharides. Any of the indicated potential carriers may be either naturally derived, wholly synthetic, or combinations of both.

The volume of carrier per volume of silk fibroin biomaterial may be increased or decreased in a range between 0% to 100% depending upon the desired physical properties of the resultant drug delivery platform including dose delivery, viscosity, injectability, and desired in vivo behavioral characteristics. This carrier is then mixed with the biomaterial until achieving a “uniform” consistency which may be termed an emulsion or suspension. More specifically, for example, a biomaterial may be passed through an 18 g needle several times to create particles, injecting back and forth between a pair of syringes, then this procedure repeated with 22 g needles affixed to 1 mL syringes. Advantages derived from adding a carrier to a biomaterial or biomaterial particles include decreased viscosity in the extracellular in vivo microenvironment; release of local mechanical stress loading after drug delivery platform administration; and improved ionic composition resulting in improved biocompatibility.

A drug delivery platform disclosed herein may be formulated using material processing constraints such as silk concentration and saline concentration to tailor material longevity in vivo. In one example, a silk biomaterial might be tailored for a persistence of five weeks to six weeks in vivo by using a 1%-3% (w/v) silk gel with 25%-50% (v/v) saline carrier. In another example, a silk biomaterial might be tailored for a persistence of two months to three months in vivo by using a 3%-5% (w/v) silk gel with 20%-40% (v/v) saline. In another example, a silk biomaterial might be tailored for a persistence of 5-6 months by using 4-6% (w/v) silk gel with 20-40% (v/v) saline. In another example, a silk biomaterial might be tailored for a persistence of 7-10 months by using a 6-8% (w/v) silk gel with 20-30% (v/v) saline. The persistence of these biomaterials might also be increased or decreased by increasing or decreasing particle size respectively.

Aspects of a drug delivery platform disclosed herein may, or may not, further comprise a solubilizing component. The use of such a solubilizing component is advantageous to provide any relatively quick release of the pharmaceutically-active drug into the body for therapeutic effectiveness. Such solubilizing component should be physiologically-acceptable. In one embodiment of the present drug delivery platform, an effective amount of a solubilizing component is provided to solubilize a minor amount, that is less than 50%, for example in a range of about 1% or about 5% to about 10% or about 20% of the pharmaceutically-active drug. For example, the inclusion of a cyclodextrin component, such as β-cyclodextrin, sulfo-butylether β-cyclodextrin (SBE), other cyclodextrins and the like and mixtures thereof, at about 0.5 to about 5.0% (w/v) solubilizes about 1% to about 10% of the initial dose of a drug. This presolubilized fraction provides a readily bioavailable loading dose, thereby avoiding any delay time in therapeutic effectiveness.

Aspects of a drug delivery platform disclosed herein may, or may not, further comprise a sustained release component. Sustained release components, include, without limitation, polymers (in the form for example of gels and microspheres), such as, e.g., poly (D,L,-lactide) or poly(D,L-lactide co-glycolide), in amounts effective to reduce local diffusion rates and/or corticosteroid particle dissolution rates. The result is a flatter elimination rate profile with a lower Cₘₐₓ and a more prolonged therapeutic window, thereby extending the time between required injections for many patients. Any suitable, preferably conditionally acceptable, release component may be employed.

The sustained release component is preferably biodegradable or bioabsorbable in the body so that no residue remains over the long term. The amount of the delayed release component may very over a relatively wide range depending, e.g., on the specific sustained release component is being employed, the specific release profile desired and the like factors. Typical amounts of delayed release components, if any, included in the present drug delivery platforms are in a range of about 0.05% (w/v) to 0.1% (w/v) to about 0.5% (w/v) or about 1% (w/v) or more (weight of the ingredient in the total volume of the platform).

Aspects of the present specification provide, in part, a method of treating a condition of an individual by administering a drug delivery platform disclosed herein. As used herein, the term “treating,” refers to reducing or eliminating in an individual a symptom of a condition or delaying or preventing in an individual the onset of a symptom of a condition. For example, the term “treating” can mean reducing a symptom of a condition by, e.g., at least 20%, at least 30%, at least 40%, at least 50%, at least 60%, at least 70%, at least 80%, at least 90% or at least 100%. The effectiveness of a compound disclosed herein in treating a condition can be determined by observing one or more symptoms and/or physiological indicators associated with the condition. An improvement in a condition also can be indicated by a reduced need for a concurrent therapy. Those of skill in the art will know the appropriate symptoms or indicators associated with specific condition and will know how to determine if an individual is a candidate for treatment with a drug delivery platform disclosed herein.

A drug delivery platform is administered to an individual. An individual is typically a human being. The condition to be treated includes, without limitation, glaucoma and/or elevated intraocular pressure corneal haze and/or opacity, inflammatory bowel disease, a wound, a soft tissue condition. The compounds represented by formula I are useful in treating glaucoma, corneal hazing or opacity, inflammatory bowel disease and a soft tissue condition, lowering intraocular pressure, and promoting wound healing. See e.g., Donde, et el., 10,10-Dialkyl Prostanoic Acid Derivatives as Agents for Lowering Intraocular Pressure, U.S. Pat. No. 6,875,787; Donde, et el., 10,10-Dialkyl Prostanoic Acid Derivatives as Agents for Lowering Intraocular Pressure, U.S. Patent Publication 2004/0235958; Donde, et al., Treatment of Inflammatory Bowel Disease, U.S. Patent Publication 2005/0164992, Jiang, et al., Compositions and Methods for Skin Repair, U.S. Provisional Patent Application 61/374,439; and Jiang, et al., Compositions and Methods for Treating Corneal Haze, U.S. Provisional Patent Application 61/369,232; Dennis Van Epps, et al., Compositions and Improved Soft Tissue Replacement Methods, U.S. Patent Application 61/375,144, each of which is hereby incorporated by reference in its entirety.

The route of administration of composition administered to an individual patient will typically be determined based on the cosmetic and/or clinical effect desired by the individual and/or physician and the body part or region being treated. A drug delivery platform may be administered by any means known to persons of ordinary skill in the art including, without limitation, syringe with needle, catheter, orally, topically, or by direct surgical implantation.

The amount of a compound included in a drug delivery platform disclosed herein will typically be a therapeutically effective amount. As used herein, the term “therapeutically effective amount” is synonymous with “effective amount”, “therapeutically effective dose”, and/or “effective dose” and refers to the amount of compound that will elicit the biological, cosmetic, or clinical response being sought by the practitioner in an individual in need thereof. As a non-limiting example, an effective amount is an amount sufficient to reduce a symptom associated with glaucoma, reduce elevated intraocular pressure, reduce a symptom associated with inflammatory bowel disease, promote wound healing, or reduce a symptom associated with corneal haze or opacity. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

In aspects of this embodiment, the amount of a compound included in a drug delivery platform is, e.g., 0.01 mg, 0.05 mg, 0.1 mg, 0.5 mg, 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 500 mg, 750 mg, or 1000 mg. In other aspects of this embodiment, the amount of a compound included in a drug delivery platform is, e.g., 0.01 mg/mL, 0.05 mg/mL, 0.1 mg/mL, 0.5 mg/mL, 1 mg/mL, 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 500 mg/mL, 750 mg/mL, or 1000 mg/mL. In yet other aspects of this embodiment, the amount of a compound included in a drug delivery platform is, e.g., about 0.01 mg to about 0.1 mg, about 0.1 mg to about 1 mg, about 1 mg to about 10 mg, about 10 mg to about 100 mg, or about 100 mg to about 1000 mg. In still other aspects of this embodiment, the amount of a compound included in a drug delivery platform is, e.g., about 0.01 mg/mL to about 0.1 mg/mL, about 0.1 mg/mL to about 1 mg/mL, about 1 mg/mL to about 10 mg/mL, about 10 mg/mL to about 100 mg/mL, or about 100 mg/mL to about 1000 mg/mL.

### EXAMPLES

The following examples illustrate representative embodiments now contemplated, but should not be construed to limit the disclosed purified silk fibroin and method for purifying such silk fibroins, biomaterials comprising such silk fibroin with or without an amphiphilic peptide and methods for making biomaterials comprising such silk fibroin and the use of silk fibroin biomaterials in a variety of medical uses.

### Example 1

### Silk Sericin Extraction

Silk fibroin for generation of the hydrogel was obtained in the form of degummed B. mori silk at a size of 20 denier-22 denier (38 μm±5.6 μm diameter). This degummed silk was further processed in order to remove the inherently present and potentially antigenic protein glue, sericin that conjoins independent fibroin filaments. This was done as described previously herein. Following removal of sericin, the pure fibroin was dried carefully to ambient humidity levels using a laminar flow hood.

### Example 2

### Generation of Silk Fibroin Solution

Silk fibroin filaments, cleaned of their sericin and rinsed free of insoluble debris and ionic contaminants were used for the generation of an aqueous silk solution. These silk fibers were added to a solution of 9.3M LiBr and purified water to make a solution consisting of 20% pure silk (% w/v). This mixture was then heated to a temperature of 60° C. and digested for a period of four hours. A total of 12 mL of the resultant solution was then loaded into a 3 mL-12 mL Slide-A-Lyzer dialysis cassette (Pierce Biotechnology, Inc., Rockford, Ill.) (molecular weight cutoff of 3.5 kD) and placed into a beaker containing purified water as a dialysis buffer at a volume of 1 L water per 12 mL cassette of silk solution. The beakers were placed on stir plates and stirred continuously for the duration of the dialysis. Changes of dialysis buffer occurred at 1, 4, 12, 24, and 36 hours of processing time.

Following dialysis, the solution was removed from the cassettes by means of a syringe and needle and centrifuged at 30,000 g relative centrifugal force (RCF) at 4° C. for 30 minutes, decanting the silk solution into a clean centrifuge tube, then repeating the centrifugation for a further 30 minutes. This process of centrifugation is beneficial for removal of insoluble particulate debris associated with the silk solution both prior to and following after dialysis. It is believed that such insoluble debris could serve as antigens in vivo or perhaps nucleation points about which gelation of the silk could occur, shortening storage life of the solution and compromising the uniformity of the gelation system. After completion of the second centrifugation, the supernatant was again collected and stored at 4° C. until needed. To confirm uniformity of the dialysis product, known volumes of the solution were collected, massed, and then dried completely through lyophilization. These lyophilized samples were then massed and the dry mass of solution compared to initial solution volume to determine percent silk present per unit volume of solution. Additionally, the solution was assessed via X-ray Photoelectron Spectroscopy (XPS) analysis to ensure that no detectable quantities of Li⁺or Br ions were present in the solution.

### Example 3

### Induction of Gelation

A variety of different methods were employed in the course of hydrogel development for the purposes of contrasting and comparing certain relevant properties of various formulae. Regardless of the nature in which the gelation process was carried out, the final determination that a “gel” state had been reached was applied uniformly to all groups. A solution or composite of solutions (i.e., silk solution blended with an enhancer or enhancer solution) was considered a gel after observing formation of a uniform solid phase throughout the entire volume, generally opaque and white in appearance.

Samples to be produced by passive gelation were not exposed to any enhancer additives. These gels were produced by measuring a volume of silk solution into a casting vessel, for the purposes of these experiments, polypropylene tubes sealed against air penetration and water loss, and the sample allowed to stand under ambient room conditions (nominally 20-24° C., 1 atm, 40% relative humidity) until fully gelled. Care was taken to ensure uniformity of casting vessels material of construction across groups so as to avoid potential influence from surface effects. These effects may serve to enhance or inhibit gelation and may be caused by factors including but not limited to siliconization, surface roughness, surface charge, debris contamination, surface hydrophobicity/hydrophilicity, and altered mass transfer dynamics.

Samples produced by means of a 23RGD-induced process were made in one of two ways, the first being direct addition of 23RGD in a pre-determined ratio to the silk solution without any sort of reconstitution. The 23RGD (obtained as a desiccated fine powder form) was blended into a measured volume of 8% silk solution within the casting vessel by pipetting using a 1000 μL pipette. These gels were then cast in polypropylene tubes, sealed against air penetration and water loss, and the sample was allowed to stand under ambient room conditions (nominally 20-24° C., 1 atm, 40% relative humidity) until fully gelled.

The 23RGD-induced gels were also produced by first dissolving the 23RGD powder in purified water. The concentration of this solution was determined based upon the amount of 23RGD to be introduced into a gel and the final concentration of silk desired in the gel. In the case of 4% silk gels enhanced with 23RGD, quantities of water equal to the amount of 8% silk solution to be used in the gel were used for the dissolution of appropriate quantities of 23RGD. In the case of gels induced by addition of 23RGD to be generated at a molar ratio of 3:1 23RGD:silk, a quantity of 23RGD was dissolved in 1 mL of water per 1 mL of 8% silk solution to be gelled. This mixing was performed in the casting vessel as well, being accomplished by means of rapid pipetting with a 1000 μL pipette when appropriate. These gels were then cast in polypropylene tubes, sealed against air penetration and water loss, and the sample was allowed to stand under ambient room conditions (nominally 20-24° C., 1 atm, 40% relative humidity) until fully gelled.

Samples produced by means of ethanol-enhanced gelation (EEG) were generated by means of directly adding ethanol to a measured volume of 8% silk solution in the casting vessel. The ethanol is added in a quantity such that the volume added should yield a volumetric dilution of the 8% silk solution resulting in the final required concentration of silk within the gel, assuming minimal volume loss due to miscibility of the organic added. The mixture of ethanol and silk solution is then mixed by means of pipetting with a 1000 μL pipette when appropriate. These gels were then cast in polypropylene tubes, sealed against air penetration and water loss, and the sample was allowed to stand under ambient room conditions (nominally 20-24° C., 1 atm, 40% relative humidity) until fully gelled.

Samples produced by a combined 23RGD-ethanol effect (RGDEEG) were generated using a solution of 90% ethanol, 10% purified water and appropriate quantities of 23RGD dissolved in this solvent. It was not possible to readily dissolve 23RGD in pure ethanol and it was believed that undissolved 23RGD might cause poor distribution of the peptide throughout the gel phase. As a result, it was determined that since a solution of ethanol and water offering similar gelation acceleration characteristics to a pure ethanol solution and reasonable 23RGD solubility would be an acceptable alternative. A solution of 90% ethanol and 10% water met both of these criteria and as a result was used for generation of these gels. The 23RGD concentration of this ethanol solution was determined based upon the amount of 23RGD to be introduced into a gel and the final concentration of silk desired in the gel. In the case of 4% silk gels enhanced with 23RGD, quantities of 90% ethanol equal to the amount of 8% silk solution to be used in the gel were used for the dissolution of appropriate quantities of 23RGD. In the case of gels induced by addition of 23RGD to be generated at a molar ratio of 3:1 23RGD:silk, a quantity of 23RGD was dissolved in 1 mL of 90& ethanol per 1 mL of 8% silk solution to be gelled. This mixing was performed in the casting vessel as well, being accomplished by means of rapid pipetting with a 1000 μL pipette when appropriate. These gels were then cast in polypropylene tubes, sealed against air penetration and water loss, and the sample was allowed to stand under ambient room conditions (nominally 20-24° C., 1 atm, 40% relative humidity) until fully gelled.

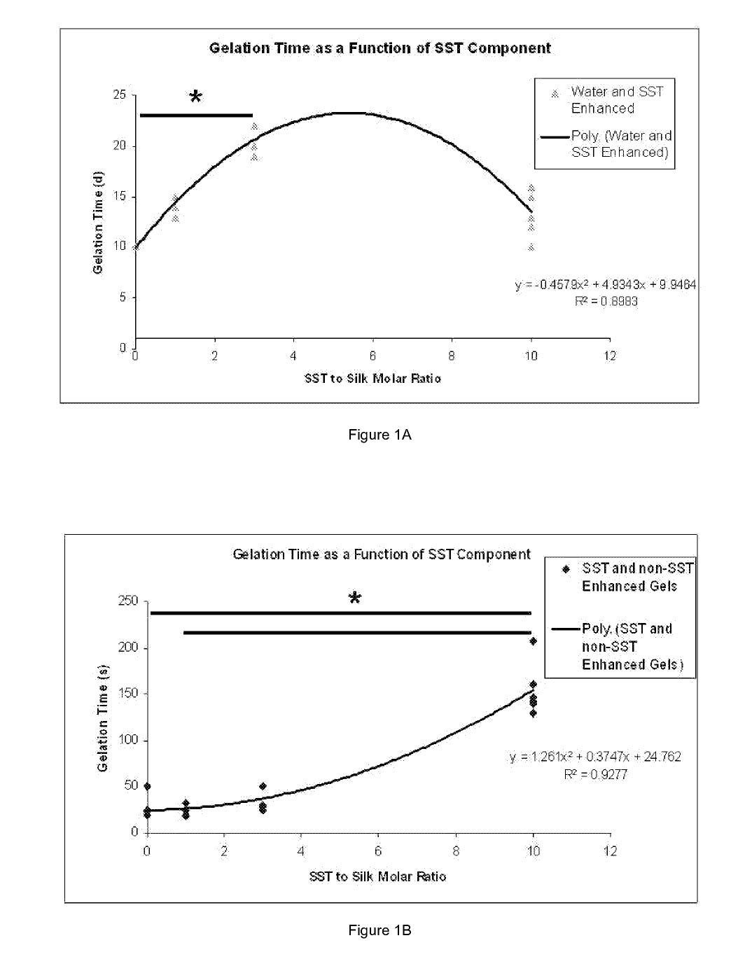

Silk gelation times were determined by casting gels according to the methods above, the exception being that gels were mixed not through pipetting, but through vigorous mechanical shaking. These studies were conducted using 1.5 mL microcentrifuge tubes as casting vessels with sample groups of N=6 used for each gel formulation (FIG. 1). The determination that a “gel” state had been reached was made in the method as described above, based upon observation of a uniform solid phase throughout the entire volume, generally opaque and white in appearance.

Gelation time varied widely depending on specific formulation. The 8P silk samples took 21 days until gelation while the 4P samples required 31±1 day (data not shown). EEG samples gelled significantly faster than PG samples with a 4E sample requiring 27±5.4 sec for gelation (p≦0.05). EEG samples gelled more rapidly as the concentration of ethanol added increased with time required gelation times of 1770±600 sec, 670.3±101.0 sec, 29.8±5.2 sec, 9.7±2.0 sec, and 4.2±0.8 sec for 6.4E, 6E, 4.8E, 4E, and 3.2E respectively. There were significant differences between all times except 4.8E and 4E, 4E and 3.2E, and 4.8E and 3.2E. RGDEEG gels generated a tightly localized white fibrous precipitate instantaneously upon addition of the ethanol solution to the silk and gelled more quickly than PG samples, though they were slower than EEG gels. 4RL, 4RM and 4RH samples took 22.7±2.5 sec, 38.8±4.5 sec, and 154.5±5 sec to gel with 4RH differing significantly from the other RGDEEG formulations.

Gelation timing experiments revealed the time constraints posed by the PG method. Results indicated that, while increased silk concentration decreased gelation time, the total time to gel was decreased only from 31 days for 4P to 21 days for 8P. This may result from the increased frequency of collisions between silk molecules in solution and resultant gel network assembly. Using ethanol directly added to silk solution as an accelerant proved to dramatically decrease the gelation time of the silk by increasing the volume of ethanol added in a fashion well-modeled by a power function. This increasingly rapid gelation is likely caused by greater competition for hydrating water molecules between silk and ethanol coupled with altered electronegativity of the solution, both favoring forced aggregation of the silk molecules. Studies conducted on RGDEEG samples revealed that addition of greater concentrations of RGD led to increasing gelation times modeled by an exponential function. This appears counter-intuitive as it was expected that RGD should function in some capacity to accelerate gelation.

The slowing of gelation in RGDEEG samples may result from difficulties in silk molecular binding to the RGD-coated silk precipitates, perhaps due to stearic interference with hydrophobic regions of silk chains. Upon RGD-ethanol accelerant addition to the silk solution, a large quantity of silk-RGD complexes was precipitated from the solution. It was noted during the gelation of RGDEEG samples that a fibrillar, white, opaque precipitate was consistently formed within the solution mixture immediately upon mixing. This precipitation from solution may be evidence of this rapid assembly of high concentration silk-RGD precipitates. This formation may be caused by association between silk micelles and peptide molecules in solution, disruption of the silk micelles, and rapid assembly of them into a tightly-localized fibrillar structure. This rapid assembly may progress until driving gradients generated by the differing solvent chemistries provided by the ethanol and water reach an equilibrium state. At this point, silk molecules are able to remain stably in solution with further silk network assembly occurring only by slow lengthening of the initially formed precipitates. While this precipitation provided a high number of nucleation points to initiate completion of a gel network, these nucleation points may be of limited utility based upon availability of binding sites. The remaining silk molecules were much slower to assemble as a result. These precipitates also tended to initiate assembly of a peripheral network comprised largely of loose α-helix and random coil motifs, possibly due to interference in silk packing due to the interference of these particles.

The hydrogels produced by the methods described above derive substantial benefit from the ability to more precisely control the time course for its gelation in comparison to that of a conventionally designed and cast gel. It is evident from monitoring the time between casting and gelation of the device and similarly cast, non-enhanced or exclusively ethanol modified gels that 23RGD under certain circumstances may be manipulated to have an additional accelerant effect upon the process of gelation. This observed enhancer effect both mitigates the time constraints and controllability associated with non-modified gels and additionally alters the manner in which the protein aggregate network is formed relative to solely ethanol enhanced gels

### Example 4

### Determination of Residual Ethanol by Colorimetric Analysis

Following gelation of a sample produced with an ethanol or 23RGD component, the gel was removed from the casting vessel and immersed in a bulk of purified water as a rinse buffer. This bulk comprised a volume such that the volumetric ratio of water to gel was 100:1. The gel was permitted to lay static in the rinse buffer for a period of 72 hours, changing the water every 12 hours.

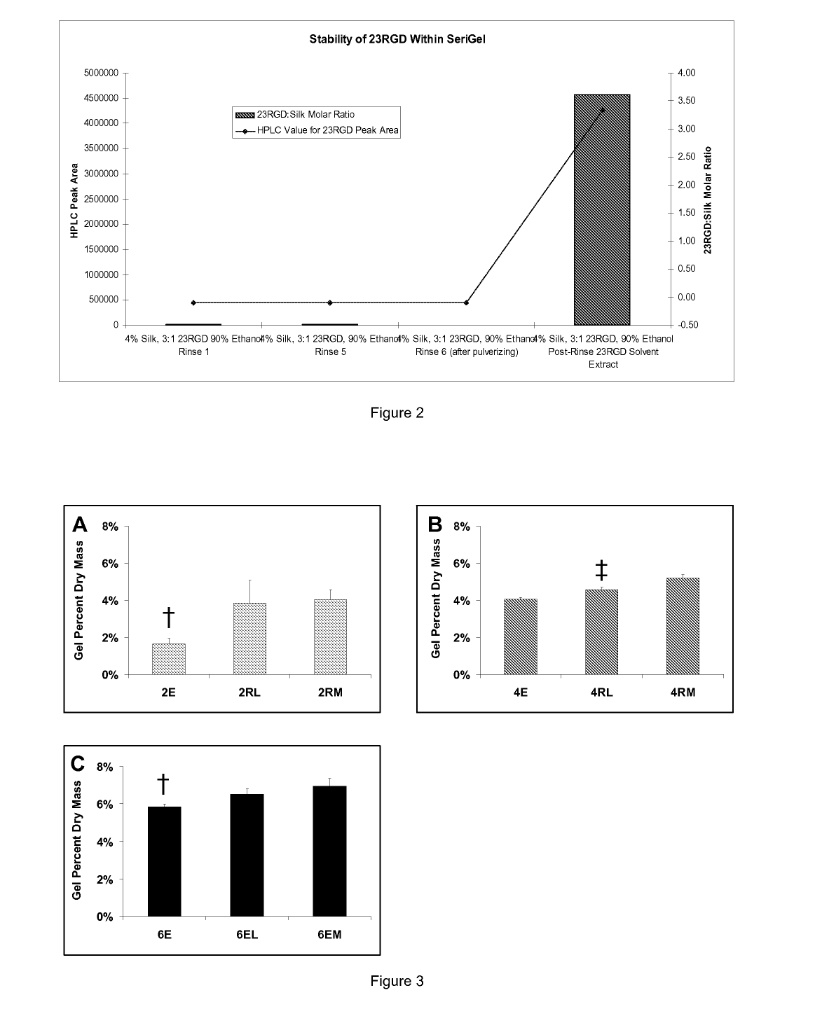

Samples of silk gel were evaluated to determine the total residual content of ethanol in a series of 23RGD-ethanol- and ethanol-enhanced gels. Briefly, samples of gel (N=4 of each type) generated as described above were processed and analyzed using an Ethanol Assay Kit (kit # K620-100 from BioVision Research Prods, Mountain View, Calif.). Samples of gel were cut to a size of approximately 0.3 cm in height by 0.5 cm in diameter (approximately 250 mg). These samples were massed to the nearest 0.1 mg using an APX-60 (Denver Instrument, Denver Colo.) balance as per the manufacturer's instructions. These gel samples were individually ground using a metal spatula and placed into 250 μL of Milli-Q water in microcentrifuge tubes. These gels were incubated at 37° C. for a period of 24 hours. After incubation, the gels were centrifuged on an Eppendorf 5415 microcentrifuge with an HA 45-18-11 rotor (Hamburg, Germany) at 18,000 rpm for 30 minutes. At the conclusion of this centrifugation step, the supernatant was used as the sample of interest according to the instructions provided by the kit manufacturer. Colorimetric analyses of the sample was performed at an absorbance of 570 nm using a spectrophotometer, and in conjunction with a standard curve, residual percentages of ethanol in the gel were calculated (Table 1, FIG. 2). It was shown in this process that the leeching step is capable of substantially removing residual ethanol from the silk gels, as none of these materials exhibited a residual ethanol component of greater than 5% ethanol by mass.

TABLE 1
Determination of Residual Ethanol by Colorimetric Analysis

| Silk | | | Initial | Final Ethanol | |
|---|---|---|---|---|---|
| Concen- | Enhancer | Enhancer | Ethanol | Concentration | |
| tration | Solvent | Solute | Concentration | Mean | Stdev |
| 2% | 90% | None | 68% | 2.49% | 0.06% |
| | | 3:1 23RGD:Silk | | 4.44% | 0.13% |
| | | 10:1 23RGD:Silk | | 4.77% | 0.29% |
| 4% | | None | 45% | 2.55% | 0.07% |
| | | 3:1 23RGD:Silk | | 2.86% | 0.08% |
| | | 10:1 23RGD:Silk | | 2.97% | 0.07% |
| 6% | | None | 22.5% | 3.12% | 0.05% |
| | | 3:1 23RGD:Silk | | 3.16% | 0.04% |
| | | 10:1 23RGD:Silk | | 2.99% | 0.10% |

### Example 5

### 23RGD Quantification by HPLC

23RGD-infused gels were studied to quantify the amount of 23RGD bound to the silk-hydrogel device as well as the quantity of free 23RGD which might be rinsed free of the device under relevant conditions. Briefly, samples of 23RGD-infused gel were cast and rinsed according to the methods above, with samples of rinse buffer being collected from each rinse for subsequent analysis by HPLC. Additionally, subsequent to the last rinse, the gel samples were mechanically pulverized by means of a stainless steel stirring rod and the adsorbed 23RGD removed by incubation for 4 hours in a dissolving buffer. This mixture of gel and solvent was then centrifuged at 16,000 g RCF for 30 minutes. The supernatant was collected and centrifuged another 30 minutes at 16,000 g RCF after which time the supernatant was collected in a sample vial for HPLC analysis. Samples of rinse buffer from the first and last rinse were centrifuged in the same fashion after being diluted with the same solvent the gel was extracted with in a volumetric ratio of 1 part rinse buffer to 4 parts solvent. To ensure 23RGD-hydrogel device rinse-exposed surface area was not a limiting factor, the same rinse and extraction process was performed upon devices pulverized after gelation and before rinsing. The peak area consistent with 23RGD for each HPLC sample was taken and these data compared against a standard curve generated for 23RGD on the same HPLC unit under identical handling and run conditions.

The resultant data indicated levels of signal from 23RGD in samples collected from rinse buffer were just slightly higher than values for 23RGD solvent alone and were immeasurable by the standard curve, expected to resolve a relative 23RGD:silk ratio of 0.05:1. By comparison, the assay was able to detect a ratio of 3.35:1 in the final rinsed and extracted 23RGD-enhanced gel.

HPLC data confirmed complete retention of RGD on the silk hydrogel material after the rinse process. This provides not only a functional RGD component to this specific series of hydrogel formulations, but indication for use of amphiphilic peptides as candidates for introduction of other components into silk gels. This knowledge might be applied to a number of other biologically active peptide sequences, though additional work must be done to understand how these specific peptides might influence gelation and how gelation in turn impacts the functionality of these peptides.

### Example 6

### Silk Gel Dry Massing

Silk gel samples of various 23RGD-ethanol- and ethanol-enhanced formulations were cut into sample cylinders (N=4 of each type) of approximately 0.7 cm in height by 0.5 cm in diameter (approximately 500 mg). These samples were massed to the nearest 0.1 mg using a balance and placed into massed microcentrifuge tubes. After this, the samples were frozen to −80° C. for 24 hours. At the conclusion of this time, the samples were placed into a lyophilizer unit for a period of 96 hours to remove all water content. Following the completion of this 96 hour drying, the remaining protein components of the silk gel samples were massed again and the mass fraction of water in the samples determined.

Gel dry massing showed an increasing percentage of dry mass as RGD component increased in each silk concentration group (FIG. 3). The dry mass of 2E was significantly less than 2RL and 2RM (p≦0.05) at 1.63±0.30%, 3.85±1.23% and 4.03±0.53% respectively (FIG. 3A). The dry masses of 4E, 4RL and 4RM all differed significantly from each other at 4.05±0.10%, 4.56±0.12%, and 5.19±0.18% respectively (FIG. 3B). The dry mass of 6E was significantly less than both 6RL and 6RM at 5.84±0.15%, 6.53±0.28%, and 6.95±0.40% respectively (FIG. 3C).

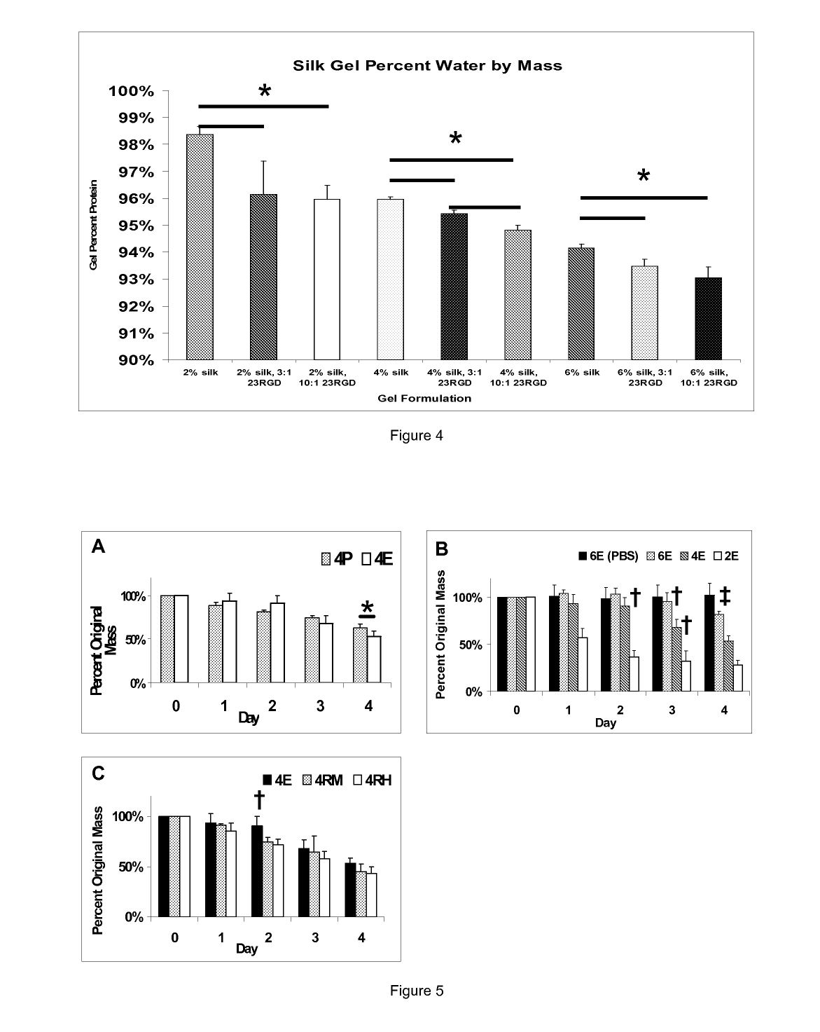

The gels, regardless of the silk concentration, showed a statistically significant trend toward decreasing percentage of water mass in each gel material as 23RGD component increased as determined by analysis of each silk concentration group with ANOVA (FIG. 4, Tukey post hoc, p<0.05). This phenomenon might be explained by the possibility that the 23RGD causes formation of a different secondary structure within the silk hydrogels and that this structure might be less hydrophilic than non-23RGD-enhanced material. It is possible that this may be manifested in a different ratio of β-sheet structure, α-helix structure, and unordered random coil for 23RGD-treated materials than their counterparts, tending to favor the more hydrophobic β-sheet conformation.

Silk gel dry mass data revealed that increasing concentrations of both silk and RGD in the silk gels increased the percentage of dry mass in these materials, though the increase from RGD was too large to attribute solely to additional peptide mass. This phenomenon might be explained by the hypothesized structure of the RGDEEG gels mentioned previously relative to PG and EEG gels. It is likely that the large regions of poorly-associated β-sheet structure in the RGDEEG gels do a poor job at integrating water into the structure. The inter-connecting regions of α-helix structures and unordered random coil are able to entrain water, but do so with less success than in the case of the more homogenous EEG gels. It may also be possible that the hydrophilic RGD sequence interfered with the dry massing procedure, causing rapid gain of water mass upon exposure of the samples to atmospheric conditions.

### Example 7

### Enzymatic Bioresorption

Gels specified were subjected to in vitro digestion by a solution consisting of non-specific protease mixture. Briefly, gel samples were cast to generate uniform, cylindrical samples of approximately 1 gram total weight. These samples were digested with a protease from Streptomyces griseus (Sigma No. P-5147) suspended in phosphate buffered saline at a concentration of 1 mg/ml. A ratio of 3 mL of protease solution per 1 ml of initial gel was used for the purposes of this study. The protease solution was added to a sealed tube containing the gel and incubated for 24 hours at 37° C. with no mechanical mixing. After 24 hours, the solution was drained through a piece of 316 stainless steel woven wire cloth. This permitted retention of all gel particles greater than 50 μm in diameter (gap size was 43 μm by 43 μm), those smaller than that were considered to be “bioresorbed” for the purposes of this assay. After thorough draining of the solution, the mass of the gel was measured wet, but devoid of excess entrained moisture. The protease solution was then replaced and the sample incubated a further 24 hours at 37° C. This process was repeated until the samples were bioresorbed for a total of four days, changing solutions and massing each day.

PG samples and EEG samples bioresorbed similarly, differing significantly only at D4 where 4P samples retained 62.89±4.26% of the original mass and 4E samples retained 53.27±5.45% (p≦0.05) (FIG. 5A). 6E gels incubated in PBS showed no significant mass loss over the course of the 4 day incubation (FIG. 5B). EEG silk gels with high concentrations of fibroin exhibited higher mass retention than lower concentrations at all days. At Day 1 there were significant differences between 2E and all other gel types with 2E, 4E and 6E gels retaining 57.04±10.03%, 93.21±9.47%, and 103.98±3.65%, respectively while 6E in PBS retained 101.18%±12.01%. At Day 2, there were significant differences again between 2E and all other gel types with 2E, 4E and 6E gels retaining 36.59±7.07%, 90.60±9.24%, and 103.24±6.38% of the original mass while 6E in PBS retained 98.28%±12.38%. At Day 3 there were significant differences between all gel types in protease, with 2E, 4E and 6E gels retaining 32.36±10.48%, 67.85±8.82%, and 95.51±8.97% of the original mass. 6E samples incubated in PBS did not differ from those incubated in protease, retaining 100.39%±12.73% of the original mass. At Day 4 there were significant differences between all gel types with 2E, 4E, and 6E gels retaining 28.14±4.75%, 53.27±5.45%, and 81.76%±3.35% of the original mass while 6E in PBS retained 102.45±12.50%. Addition of RGD to silk gels appeared to slightly decrease the mass retention of these materials when subjected to proteolytic bioresorption (FIG. 5C). 4E samples retained significantly more mass than 4RM and 4RH at Day 2 as they retained 90.6±9.24%, 74.47±4.55%, and 71.23±6.06% of the initial masses respectively. There were no further significant differences in 4E samples relative to 4RM and 4RH samples over the course of the bioresorption assay.

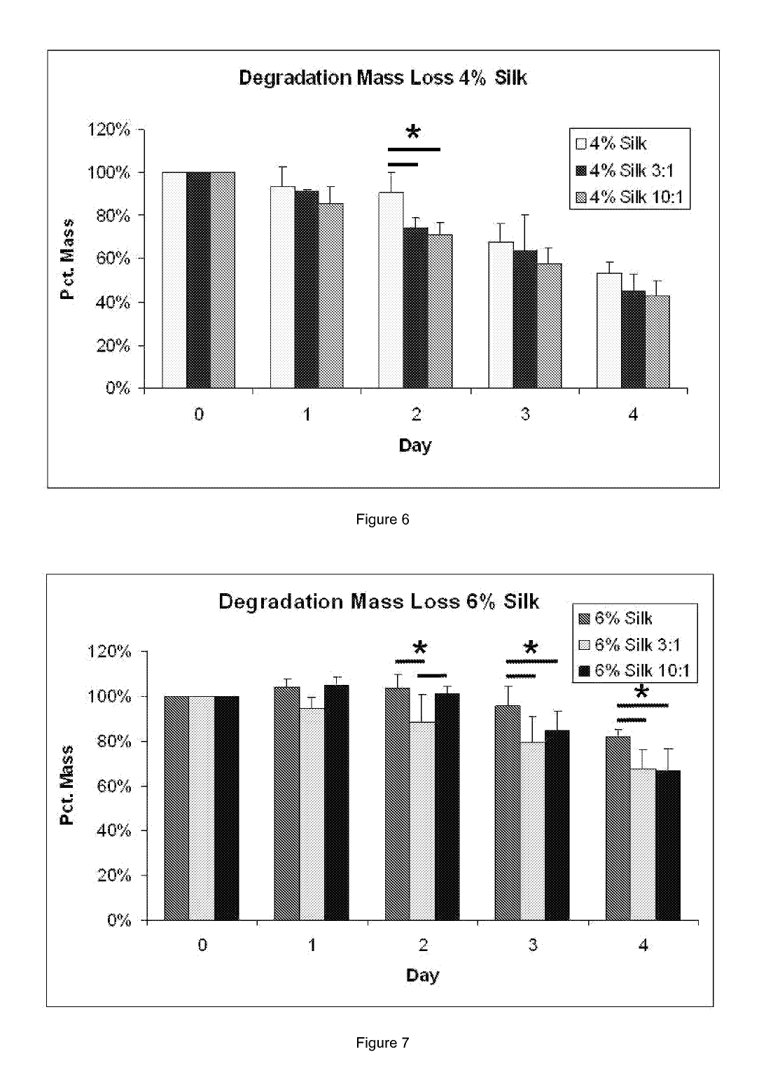

Gel samples treated with 23RGD exhibit a trend toward more rapid bioresorption within the constraints of this particular assay. This was illustrated at the 4% silk concentration (FIG. 6) and then confirmed at a concentration of 6% silk fibroin in the gel materials (FIG. 7). Significant differences in the bioresorption rates of 23RGD enhanced samples recorded by two-way ANOVA using a Bonferroni post test (p<0.05), particularly with 6% silk, reinforced the trend. The unique behavior attributed to 23RGD-enhanced materials may be due in part to its unique protein structure, as the bioresorption method considers particles below a size of 50 μm to be bioresorbed, regardless of their stability. It may be possible for a rich β-sheet structure to exist within 23RGD gels which is broken up into small, discrete regions by interfering regions of α-helix structure and random coil which bioresorb more quickly, creating a plethora of tiny, non-resorbed fragments in solution.

In vitro bioresorption of 4P and 4E samples showed both materials had a similar resistance to proteolysis (FIG. 5A). This is indicative that the basic process of ethanol-enhanced gelation is capable of generating a gel structure rapidly without sacrificing important material properties. It was also shown that increasing the concentration of silk in EEG gels from 2% to 4% to 6% in 2E, 4E, and 6E respectively, substantially decreased sample bioresorption mass loss (FIG. 5B). This may correlate to a more homogeneous, stable and resilient gel structure, or simply to a greater quantity of silk molecules to be cleaved by the proteases in order to bioresorb the samples. In either case, these data clearly indicate a potential for tailoring of bioresorption time scale of a silk gel material through alteration of the silk protein content of gels. It was also illustrated that a 4 day exposure to PBS did not appreciably alter the mass of 6E samples, providing a preliminary indication that EEG samples are not substantially degraded by hydrolysis. This is a further reinforcement of the stability and bulk integrity of these silk gels as many gel materials suffer from limited resilience in vivo due to high susceptibility to hydrolysis. Addition of increasing quantities of RGD to silk gels was shown to slightly increase the rates of bioresorption mass loss in comparing 4E, 4RM and 4RH (FIG. 5C). This behavior indicates that there may be some structural differences between RGDEEG and EEG gels which cause less mass loss in EEG gels as compared to RGDEEG in this bioresorption assay. This may relate directly to the previously proposed idea that RGDEEG materials consist of many small regions of robust β-sheet structure loosely bound together by a weak inter-connecting matrix of α-helix and unordered random coil structures. This stands in contrast to EEG materials, which are thought to assemble from similar, though less prominent and numerous, precipitates into a more homogeneous structure than RGDEEG gels as a result. The inter-connecting matrix of the RGDEEG gels is therefore more susceptible to rapid bioresorption through this proteolytic assay than that of EEG gels. While β-sheet regions may remain intact in RGDEEG gels, bulk material integrity is lost as the inter-connecting network is resorbed as are the residual β-sheet particles due to the sieving method used as a cutoff for degradation product particle size. This is indicative that it may be possible to use varying levels of RGD in order to further manipulate the structure and bioresorption profile of a silk gel.

### Example 8

### Fourier-Transform Infrared Spectrum Capture

Silk hydrogels, 23RGD-ethanol-enhanced 4% silk, 3:1 and 10:1, were cast as described above and subjected to proteolytic bioresorption as described above. Additionally, non-bioresorbed control samples were obtained for sake of analysis via FTIR in quantities of 0.5 ml each. Using a Bruker Equinox 55 spectrophotometer (Bruker Optics, Inc., Billerica, Mass.) coupled with a Pike MIRACLE™ germanium crystal (PIKE Technologies, Madison, Wis.), sample absorbance spectra were obtained. Samples were imaged by pressing them upon the crystal via a pressure arm until single sample scans indicated viable signal from the material then performing a 128-scan integration. Resolution was set to 4 cm⁻¹ with a 1 cm⁻¹ interval from a range of 4000 cm⁻¹ to 400 cm⁻¹. Resultant spectra were subjected to analysis via OPUS 4.2 software (Bruker Optics, Inc). A peak-find feature was used to identify peaks between 4000 cm⁻¹ and 600 cm⁻¹, with the search criteria being automatic selection of local inflection points of a second-derivative, nine-point smoothing function. Program sensitivity was set to 3.5% for all spectra based upon operator discretion regarding magnitude of peaks identified and likely relevance to compound identification and “fingerprinting”.

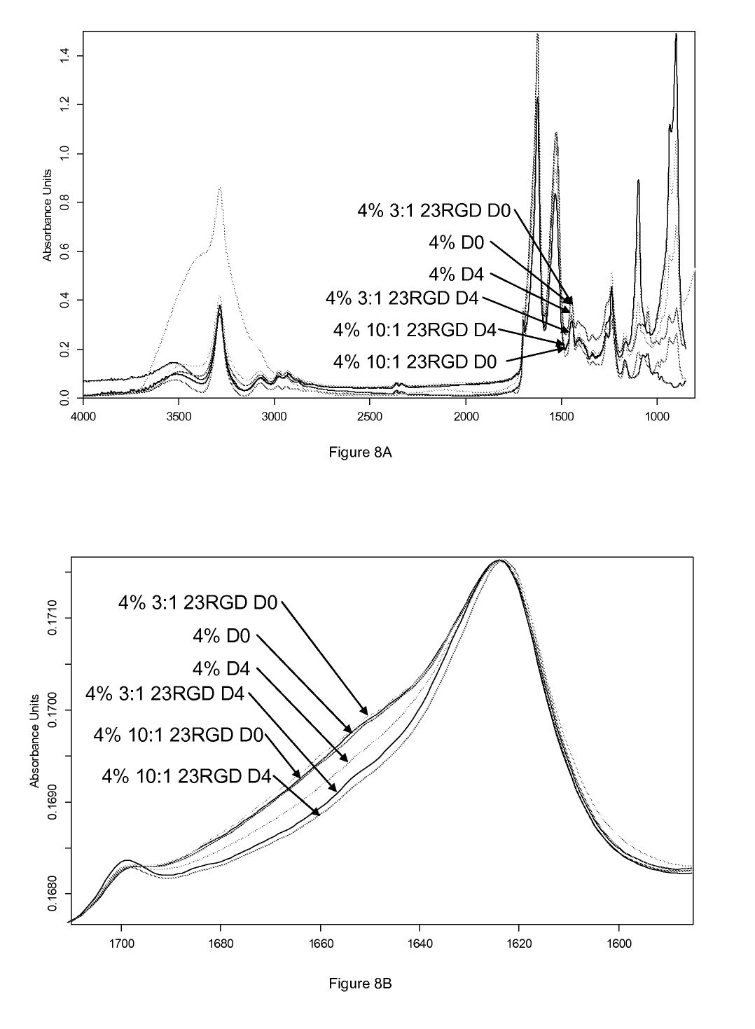

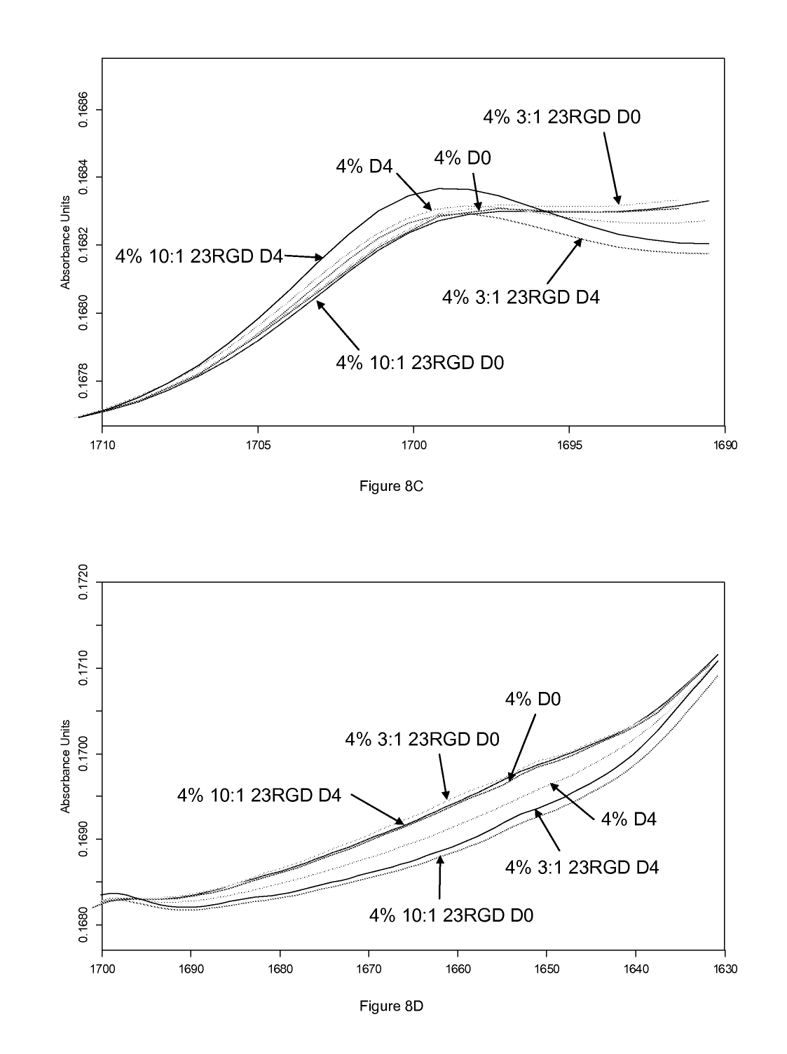

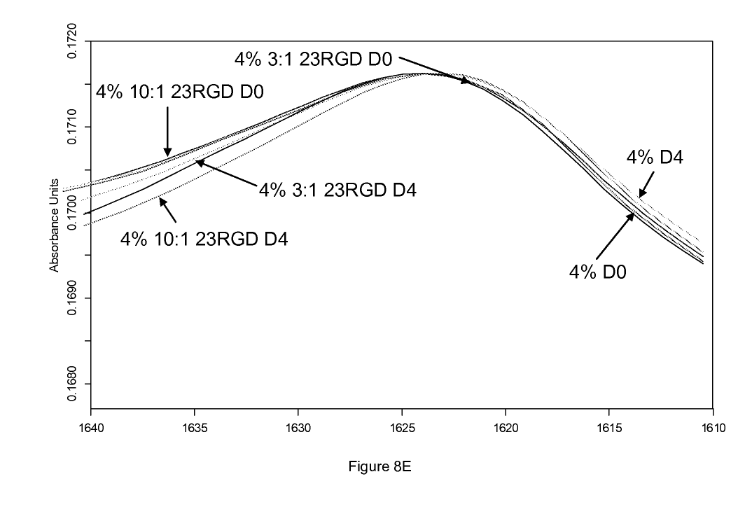

Each of the samples subjected to FTIR analysis exhibited a spectrum with very pronounced peaks at the Amide I band (1600-1700 cm⁻¹) (FIG. 8). Additionally, the specific wave numbers of these peaks are consistent between the 23RGD-infused silk fibroin hydrogel and other silk gel groups. All samples exhibit major peaks at ˜1622 cm⁻¹ and a minor peak/toe region at ˜1700 cm⁻¹, a pattern associated with a high degree of β-sheet structure within a sample (FIG. 8). There are also similarities across all samples types at the Amide II band with a major peak at ˜1514 cm⁻¹.

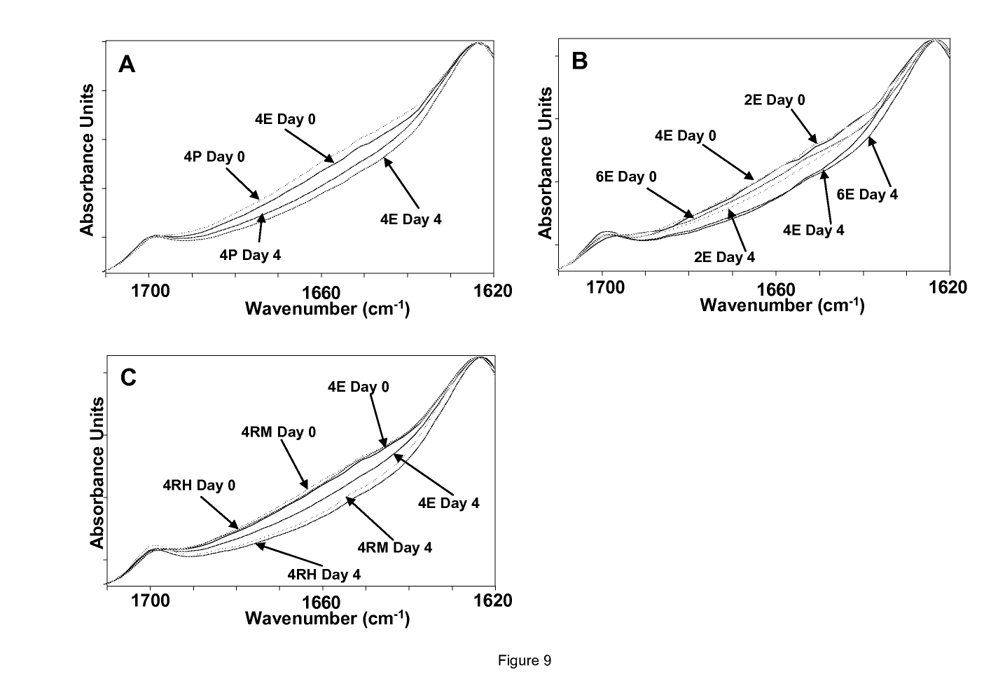

Use of the EEG process to produce silk gels did not dramatically impact gel secondary structure but did slightly increase the resistance of the gel formulation to proteolytic bioresorption (FIG. 9A). Evaluation of characteristic FTIR spectra of 4P and 4E gels at Day 0 revealed few distinguishing characteristics as both formulations exhibited a characteristic β-sheet peak around 1622 cm⁻¹ and to region of β-turn at 1700 cm⁻¹. Each sample also had additional portions of β-sheet, β-turn, α-helix, and unordered random coil at 1677 cm⁻¹, 1663 cm⁻¹, 1654 cm⁻¹, and 1645 cm⁻¹ respectively with higher relative quantities of α-helix and random coil appearing in 4P than 4E at Day 0. At Day 4, both samples showed pronounced decreases in 1677 cm⁻¹ β-sheet, β-turn, α-helix and random coil signal, though this 4P exhibited this to a greater extent than 4E, indicating preferential resorption of these motifs and greater resistance to this in 4E gels.

Increasing the final silk concentration of EEG gels had little impact on initial gel secondary structure, though there was a pronounced increase in β-sheet structures at Day 4 with greater silk concentrations (FIG. 9B). At Day 0, 2E, 4E, and 6E gels all showed strong signal for 1622 cm⁻¹ β-sheet and 1700 cm⁻¹ β-turn strong, with 6E having particularly prominent peaks in these regions. Each sample also had additional portions of 1677 cm⁻¹ β-sheet, 1663 cm⁻¹ β-turn, α-helix, and unordered random coil. At Day 4 all gels showed decreases in 1677 cm⁻¹ β-sheet, 1663 cm⁻¹ β-turn, α-helix and random coil peaks relative to 1622 cm⁻¹ β-sheet and β-turn peaks with this behavior being more marked in 4E and 6E than 2E. The Day 4 6E sample also showed a more stable β-sheet structure indicated by a peak shift to lower wave number at ˜1620 cm⁻¹.

Pronounced differences in the 23RGD-ethanol-enhanced and ethanol-enhanced spectra only became evident after a four-day period of bioresorption in protease. The day 4 samples exhibited differences primarily in the order of magnitude of certain secondary structure modalities seen through slight differences in FTIR Amide I band shape. At day 4, the 23RGD-ethanol-enhanced samples exhibit higher levels of β-turn structure evidenced by far more pronounced and distinct peaks at ˜1700 cm⁻¹ while also showing considerably lower levels of α-helix structure (1654 cm⁻¹) and unordered random coil (1645 cm⁻¹) structures. For example, FTIR spectra from 4E, 4RM and 4RH all show similar structures featuring 1622 cm⁻¹ β-sheet and 1700 cm⁻¹ β-turn prominently with indications of 1677 cm⁻¹ β-sheet, 1663 cm⁻¹ β-turn, α-helix, and unordered random coil secondary structures (FIG. 9C). At Day 4, 4RM and 4RH both show a less pronounced 1677 cm-1 β-sheet, 1663 cm⁻¹ β-turn, α-helix, and random coil component than the 4E sample with 4RH also showing a more stable β-sheet structure, indicated by a peak shift to lower wave number at ˜1620 cm⁻¹. Additionally, a peak shift occurred in both the 10:1 23RGD-ethanol-enhanced and ethanol-enhanced samples in the β-strand peak at 1622 cm⁻¹, indicative of increased β-sheet stability. Considered as a whole, the collective peak shifts and peak magnitudes observed in the spectra at day 4 compared to day 0, all gel types experienced substantial strengthening of β-sheet component, likely due to removal of less-stable α-helix and random coil. These effects were most pronounced in 23RGD-enhanced gel materials, likely due to intrinsic differences in the initial organization of the structural network of the gel materials.

FTIR analysis and comparison of PG, EEG and RGDEEG showed strong behavioral similarities across all gel groups. Each material exhibited β-sheet-dominated secondary protein structures, featuring elements of α-helical and random coil structures and each resorbed in such a fashion that the quantities of β-sheet-rich structure increased relative to α-helical and random coil structures. The selective bioresorption of α-helical and random coil structures indicates that they are likely favorably degraded by proteolysis relative to β-sheet structures, thus the bioresorption profile of a gel might be influenced by altering the balance between β-sheet motifs and the combination of α-helical and random coil structures. An evaluation of ethanol as an accelerant revealed a minimal effect on silk gel structure at Day 0 as both 4P and 4E had high β-sheet contents with α-helical and random coil structures (FIG. 9A). At Day 4 though, there was a slightly greater relative β-sheet content in 4E than 4P samples. This may be caused by structural differences in 4E and 4P formulations that were imperceptible at Day 0 by ATR-FTIR, possibly in the uniformity and homogeneity of the silk gels. It is possible that the same differences hypothesized between EEG and RGDEEG gels derived from their different extents of precipitate/nucleation point formation in early-phase gelation causes differences between PG and EEG materials as well. As PG samples are not accelerated, it is likely that very few nucleation points will form quickly and as a result, the gelation process occurs in a very slow but homogeneous fashion, allowing for an optimal stearic packing of silk molecules throughout the solution volume. This results in a consistent protein structure throughout the final gel volume, corresponding to good bulk material integrity. This would contrast with EEG gels, as the previously postulated nucleation phenomenon associated with RGDEEG materials likely occurs with EEG materials as well, though in a less prominent fashion. This results in a non-uniform distribution of highly organized regions of β-sheet held together by α-helical and random coil structures in the EEG materials relative to the PG materials, with α-helical and random coil degraded more rapidly than 6-sheet. This is in keeping with previous studies which have shown that more poorly packed β-sheet structures and α-helix structures are more susceptible to degradation. Increasing silk concentration in EEG gels from 2E to 4E to 6E revealed the most prominent β-sheet structures in 6E at both Day 0 and Day 4 while 2E had considerably more α-helix and random coil at both days than 2E and 4E (FIG. 9B). This would seem to indicate that dilute concentrations of silk in the final hydrogel result in a less densely packed secondary structure, possibly due to stearic freedom within the gel volume relative to 4% and 6% states. This indicates that silk concentration may be used to manipulate the secondary structure of silk gel to influence bioresorption. A study of the effect of increasing RGD concentration indicated that while gels were virtually identical at Day 0, the α-helix structure and unordered random coil in 4RM and 4RH gels were less resilient to bioresorption than in 4E as seen at Day 4 (FIG. 9C). This might also be explained by inhomogeneities within the 4RM and 4RH gels relative to 4E as mentioned previously. This may be particularly likely in light of the formation of precipitates observed in RGDEEG samples. This data may be indicative that RGD or a similar peptide could be used to further tailor the nature of the bioresorption profile of silk gels.

These results indicate that silk gels produced through PG, EEG, and RGDEEG result from a two-phase assembly process consisting of nucleation and aggregation. Silk gels contain predominantly β-sheet structure which is more resistant to in vitro bioresorption than α-helix and random coil. EEG gels form more quickly than PG, likely due to a more rapid precipitation and nucleation event mediated by the effects of ethanol on the solution solvent phase. EEG gels form a non-homogeneous structure likely consisting of localized, initially-precipitated β-sheet regions inter-connected by α-helix and random coil assembled subsequently. RGDEEG gels form a non-homogeneous structure likely consisting of localized, initially-precipitated β-sheet regions inter-connected by α-helix and random coil assembled subsequently. RGDEEG gels reach completion more slowly than EEG gels due to stearic RGD-mediated interference encountered in gel assembly following nucleation. RGDEEG gels are less homogeneous than EEG gels due to these difficulties associated with late-phase assembly.

### Example 9

### Injectable Gel Processing

Silk hydrogels were prepared as described above in Examples 1-4. Gels were then comminuted by grinding the silk gel to a paste using a stainless steel spatula. Gel formulations including PBS were massed with an balance and the correct volume percentage of PBS was blended in with the assumption that both the gel and PBS had a density of 1 g/ml. Silk hydrogels to be used for in vivo assessment were subjected to vigorous mechanical pulverization by means of a stainless steel stir rod. When specified as containing a saline component, gels were blended with saline at volumetric ratios based upon the original volume of gel (i.e., prior to mechanical disruption) following pulverizing by the stainless steel bar. This addition of phosphate buffered saline serves to regulate tonicity of the gel as well as improve injectability. Following this initial pulverizing, the gel was further disrupted by means of repeated injection through a 26-gauge needle in order to decrease overall particle size within the gel and improve injectability characteristics. In some samples, gel was further disrupted by means of repeated injection first through an 18 g needle repeatedly until the gel flowed readily, and then the material was then cycled in like fashion through a 23 g needle and 26 g needle.

### Example 10

### In Vivo Investigation of Silk Hydrogel in Rodent Models

Samples of silk gel which had been processed for implantation or injection in vivo as described in Example 9 were double-bagged with appropriate sterilization bags for gamma irradiation and sterilized by exposure to a dose of 25 kGy of gamma radiation.

In one trial silk hydrogel samples, both 23RGD-enhanced and native were implanted subcutaneously in male Lewis rats having an average weight of 400 g. On the day of surgery, animals were anesthetized via a ketamine/xylazine solution injected IM in the animals' hind legs. Following administration of anesthesia, the skin of the rats was shaved closely and swabbed with alcohol, allowed to dry, swabbed with BETADINE® microbicide (Purdue Pharma, Stamford, Conn.) then draped with sterile towels. In the case of implanted devices, two dorsal midline incisions were made directly over the spine, the first 0.5 cm below the shoulders and the second 2.5 cm above the pelvic crest, each 1 cm long each. The incisions were expanded into 1 cm deep pockets using a blunt dissection technique beneath the panniculus carnosus at each side yielding 4 potential implant sites. Implants, 3 per animal; each 1 cm×1 cm×0.3 cm in size were inserted into the pockets without fixation with the final site undergoing the same dissection but replacing the implant with 0.5 mL of sterile saline solution. The skin was closed with interrupted absorbable sutures. Depending on study, samples were harvested at 7 days, 14 days, 28 days, and/or 57 days after implantation surgery. Gross observations were collected semi-weekly regarding implant site appearance. After sample harvest, gross observations of the implants were conducted and samples were processed for histological evaluation. Analysis of histology slides was provided by a trained veterinary pathologist.

Sections were scored for presence (0=none, 1=present) of implant mineralization, cyst formation, fibrosis, sebaceous cell hyperplasia, and focal follicular atrophy. Additionally, the density of inflammatory response (0=none . . . 5=extensive) and extent epidermal hyperplasia (0=none . . . 3=extensive) were graded. These data were reported as percentages of the highest score possible for the group of slides. Sections were also examined for presence of any particular characteristic cell types including lymphocytes, neutrophils, eosinophils, mononuclear giant cells, macrophages, and fibroblasts. Additional commentary relevant to the host response was included at the discretion of the reviewing pathologist. Prism 4.03 (GraphPad Software Inc., San Diego, Calif.) was used to perform analysis of variance (ANOVA) with a significance threshold set at p≦0.05. One-way ANOVA was used to compare differences average extrusion forces for comminuted gels. For all tests, Tukey's post-hoc test was also performed for multiple comparisons.

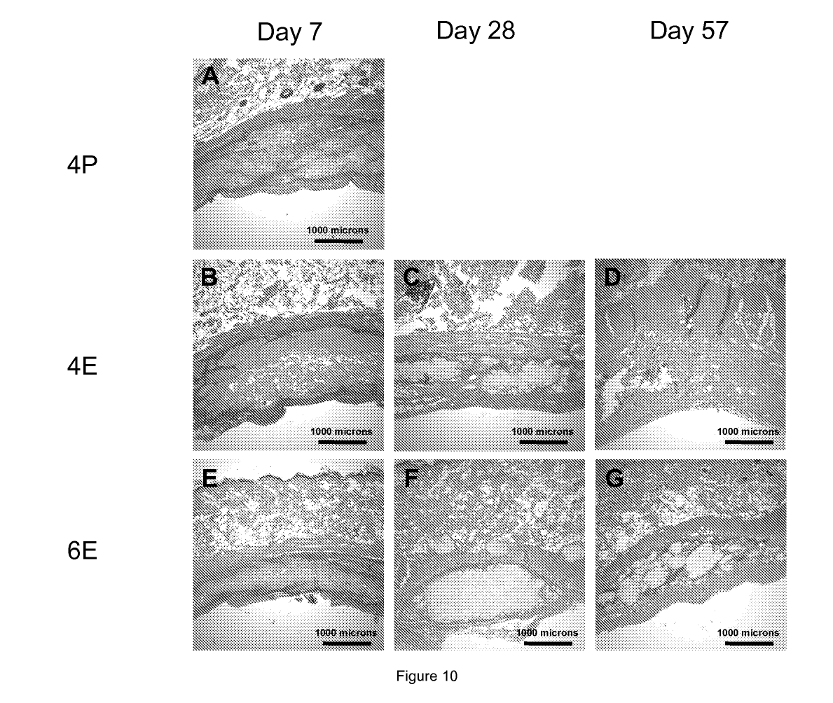

Table 2 lists the formulations of silk gel, both 23RGD-ethanol-enhanced and ethanol-enhanced developed and assessed intradermally in a rat model. Silk gels explanted from rats at Day 7 were visibly well-defined and easily identifiable with no gross indications of edema, erythema, or transdermal elimination of material. It was not possible to differentiate sites of PBS control implantation from surrounding tissue. H&E sections of 4% silk fibroin hydrogels formed by passive gelation (4P), 4% silk fibroin hydrogels formed by ethanol-enhanced gelation (4E) and 6% silk fibroin hydrogels formed by ethanol-enhanced gelation (6E) all appeared similar, with mild inflammation in all cases characterized by lymphocytes, macrophages, some neutrophils and fibroblasts (FIG. 10). Cellular infiltration was observed in all sample types with complete penetration in 4P and peripheral ingrowth to a depth of about 100 μm in both EEG gels with no evidence of cyst formation observed. In all gels, early bioresorption was indicated by implant edge erosion with residual implant material remaining localized into large lakes. Host integration of implanted gel had progressed in Day 28 samples of 4E and 6E evidenced by greater cellular ingrowth into the material with complete implant penetration in 4E samples and robust peripheral ingrowth in 6E samples. The cellular response at this time point was characterized by fibroblasts, lymphocytes and macrophages with the addition of a few multi-nucleated giant cells.

TABLE 2
Silk Hydrogel Formulations

| Group | Silk | | Saline |
|---|---|---|---|
| Name | Concentration | Enhancer | Component |
| 4E10 | 4% | 90% Ethanol | 10% |
| 4R10 | | 90% Ethanol, 1:1 23RGD | |
| 4RH10 | | 90% Ethanol, 3:1 23RGD | |
| 4E25 | | 90% Ethanol | 25% |
| 4R25 | | 90% Ethanol, 1:1 23RGD | |
| 4RH25 | | 90% Ethanol, 3:1 23RGD | |
| 6E10 | 6% | 90% Ethanol | 10% |
| 6R10 | | 90% Ethanol, 1:1 23RGD | |
| 6E25 | | 90% Ethanol | 25% |
| 6R25 | | 90% Ethanol, 1:1 23RGD | |
| 6RH25 | | 90% Ethanol, 3:1 23RGD | |

Day 57 samples of 4E and 6E showed continued host bioresorption of the gel material as there was little residual 4E and while 6E remained visible in large, intact lakes, the gel had been completely penetrated with host tissue. The host response to 4E had dramatically decreased in cellularity between Day 28 and Day 57 with very little evidence of hypercellularity at Day 57 with some scattered macrophages and fibroblasts around the implant site. The pathology of the host response of 6E was similar to the Day 28 response to 4E, with fibroblasts as the predominant cell type and scattered lymphocytes, macrophages and multi-nucleated giant cells. This was viewed as a low-grade, persistent, fibrotic-type inflammatory response to the material.

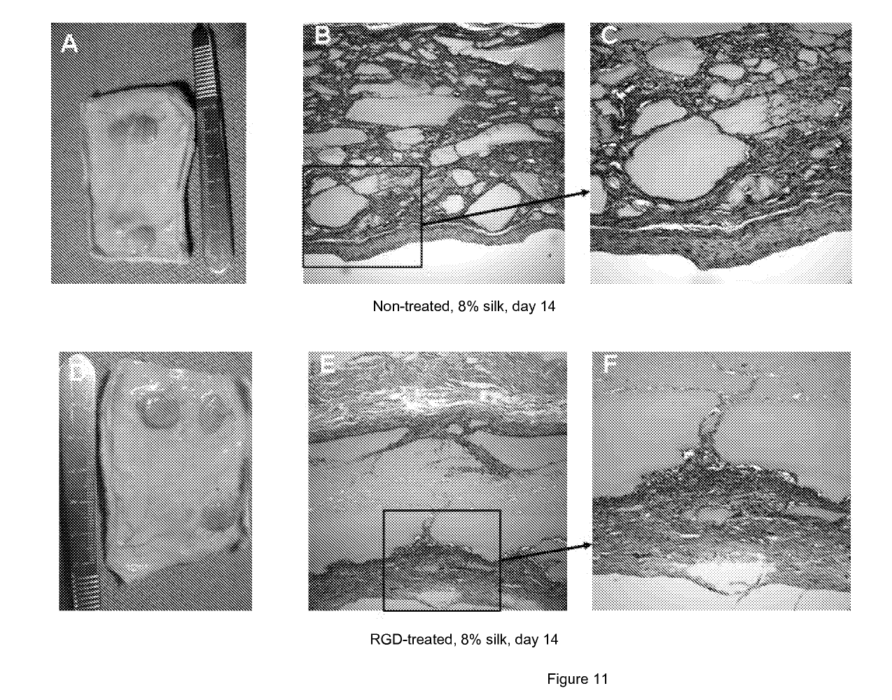

Samples of 23RGD-enhanced gel exhibited a less robust inflammatory response at the 14 day time point in comparison to non-23RGD-enhanced gel (FIG. 11). This is observed through an appreciable decrease in hyper-cellularity proximal to the gel implant and an accompanying decrease in the fragmentation of the implant material. It is possible that this improvement in implant integrity is due to a less robust foreign body response by the host animal and it may also be evidence that there is less mechanical contraction of the implant site, a commonly observed phenomenon with biomaterials including the “RGD” motif. These effects indicate that 23RGD-enhancement of silk gels leads to a more biocompatible material with better implant outcomes.

In a second trial, intradermally-injected samples of silk hydrogel, both ethanol enhanced and 23RGD-ethanol enhanced and relevant control materials were investigated using male Hartley guinea pigs. This was done according to protocol#29-05 on file with New England Medical Center's Department of Laboratory Animal Medicine (DLAM) and approved by the Institutional Animal Care and Use Committee (IACUC). Briefly, male Hartley guinea pigs weighing 300-350 g were anesthetized via a ketamine/xylazine cocktail injected intramuscularly into the animals' hind legs. The dorsal skin of the guinea pigs was then shaved closely and swabbed with alcohol, allowed to dry, swabbed with BETADINE® microbicide or Chloraprep (Enturia, Inc., Leawood, Kans.), then draped with sterile towels. A 50 μL volume of the desired material was injected through a 26 g needle at six different sites along the left side of the animal's back. Further injections of an appropriate silk gel control were made at the six contralateral sites. Explanation of the silk gels was performed at 28 days after implantation. Gross observations were collected semi-weekly regarding implant site appearance. After sample harvest, gross observations of the implants were conducted and samples were processed for histological evaluation. Analysis of histology slides was provided by a trained veterinary pathologist. Scoring and statistical analysis was performed as described above.

Table 3 lists the formulations of silk gel, both 23RGD-ethanol-enhanced and ethanol-enhanced developed and assessed intradermally in a guinea pig model in a twenty-eight day screen. Although no statistically significant differences were identified, the data for both gross observations and histology (Tables 4 and 5) indicate a general trend supporting the previous data that 23RGD-enhancement of gel improves material biocompatibility. Among sites implanted with silk gel, gross outcomes varied. Ulceration and hair loss rates were lower in groups with 25% PBS compared to 10% saline, 6% silk compared to 4% silk and RGDEEG casting as compared to just EEG casting (Table 4). Site redness rates followed a similar pattern with the exception that RGDEEG samples induced more site redness than EEG samples. All silk gels showed evidence of epidermal cyst formation, fibrosis, epidermal hyperplasia and pronounced inflammation with traces of follicular atrophy in all EEG samples. Sebaceous cell hyperplasia was present to a limited extent in all formulations with the exception of 6% silk, 10% saline, 1:1 23RGD (Table 5). This is particularly evident in the case of silk gels of 4% silk with 25% saline added and either enhanced with an ethanol-based enhancer or an 23RGD-ethanol-based enhancer, and more specifically, in the case of site ulcerations (Table 5). This material indicated strong improvements with increasing 23RGD concentration in the number of sites ulcerating throughout the course of the trial. These results are indicative that use of 23RGD in conjunction with an ethanol enhancer provides an improved outcome when compared to an ethanol enhancer alone.

TABLE 3
Silk Hydrogel Formulations

| Group | Silk | | Saline |
|---|---|---|---|
| Name | Concentration | Enhancer | Component |
| 4E10 | 4% | 90% Ethanol | 10% |
| 4R10 | | 90% Ethanol, 1:1 23RGD | |
| 4E25 | | 90% Ethanol | 25% |
| 4R25 | | 90% Ethanol, 1:1 23RGD | |
| 4RH25 | | 90% Ethanol, 3:1 23RGD | |
| 6E10 | 6% | 90% Ethanol | 10% |
| 6R10 | | 90% Ethanol, 1:1 23RGD | |
| 6E25 | | 90% Ethanol | 25% |
| 6R25 | | 90% Ethanol, 1:1 23RGD | |

TABLE 4
Gross Evaluation of Guinea Pigs

| Group | Site | Hair | | |
|---|---|---|---|---|
| Name | Redness | Loss | Palpability | Ulceration |
| 4E10 | 38% | 58% | 65% | 33% |
| 4R10 | 57% | 49% | 67% | 33% |
| 4E25 | 28% | 34% | 49% | 28% |
| 4R25 | 44% | 34% | 64% | 17% |
| 4RH25 | 50% | 23% | 66% | 6% |
| 6E10 | 63% | 52% | 68% | 33% |
| 6R10 | 78% | 51% | 68% | 22% |
| 6E25 | 33% | 31% | 69% | 11% |
| 6R25 | 56% | 30% | 68% | 13% |
| HYLAFORM ™ | 6% | 12% | 63% | 0% |
| ZYPLAST ™ | 17% | 10% | 52% | 0% |

TABLE 5
Histological Evaluation of Guinea Pigs

| | Epidermal | | | | | |
|---|---|---|---|---|---|---|
| | Cyst | | | Epidermal | Follicular | Sebaceous |
| Group Name | Formation | Fibrosis | Inflammation | Hyperplasia | Atrophy | Hyperplasia |
| 4E10 | 22% | 100% | 70% | 59% | 11% | 22% |
| 4R10 | 74% | 100% | 62% | 67% | 0% | 14% |
| 4E25 | 50% | 100% | 69% | 67% | 13% | 13% |
| 4R25 | 29% | 100% | 39% | 62% | 0% | 14% |
| 4RH25 | 14% | 100% | 64% | 50% | 0% | 43% |
| 6E10 | 44% | 100% | 70% | 56% | 11% | 33% |
| 6R10 | 25% | 100% | 63% | 38% | 0% | 0% |
| 6E25 | 30% | 100% | 60% | 40% | 10% | 20% |
| 6R25 | 29% | 100% | 64% | 33% | 0% | 14% |
| HYLAFORM ™ | 0% | 0% | 3% | 6% | 0% | 0% |
| ZYPLAST ™ | 0% | 25% | 28% | 31% | 0% | 0% |

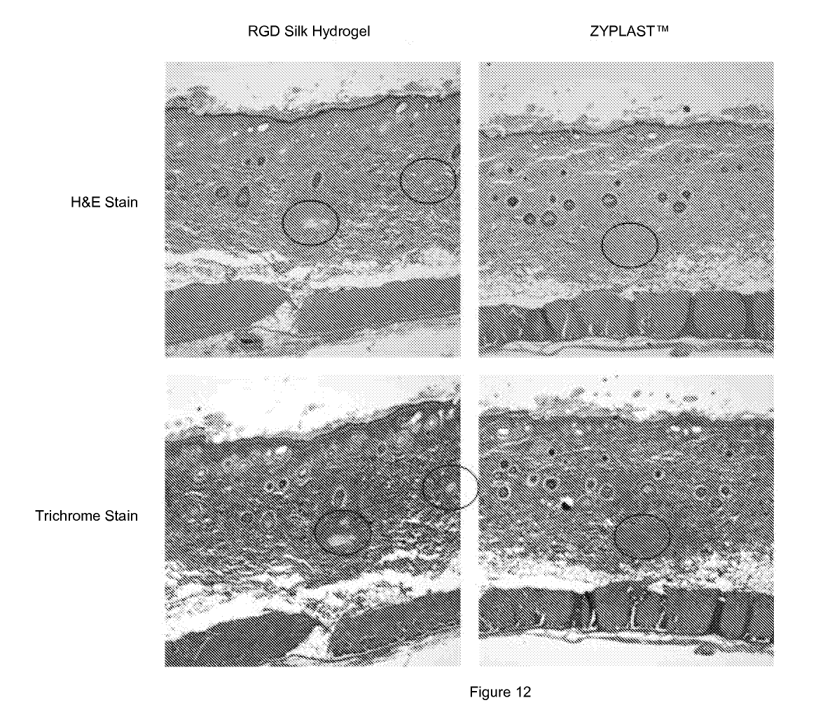

A third trial also used male Hartley guinea pigs to investigate intradermally injected samples of silk hydrogel as described above, comparing samples of 4% and 6% silk, 25% saline 3:1 23RGD-ethanol enhanced silk gels with a collagen-based control material, ZYPLAST™ (Allergan Inc., Irvine Calif.) and HYLAFORM™ (Allergan Inc., Irvine Calif.). Explanation of the silk gels was performed at 92 days after implantation. Gross observations were collected semi-weekly regarding implant site appearance. After sample harvest, gross observations of the implants were conducted and samples were processed for histological evaluation. During the course of the 92 day trial, none of the 24 implant sites, either 23RGD-ethanol-enhanced hydrogel or ZYPLAST™, ulcerated. Histology revealed that 75% of all ZYPLAST™ sites had residual material as did 75% of all 23RGD-ethanol-enhanced silk gel sites (both 4% and 6%). Both materials exhibited very similar chronic phase cellular responses, as the sites were characterized by a mild fibrotic reaction with abundant deposition of collagen in and around the implant site (FIG. 12). The collagen appears less ordered than does that in the surrounding dermal reticulum based upon the color density when viewed with Trichrome staining and also when viewed under polarized light. Silk gel sites had similar palpability scores to both control materials but exhibited higher rates of site redness, hair loss and ulceration than did ZYPLAST™ and HYLAFORM™. These results not only reinforce that 23RGD-ethanol-enhanced silk gel is biocompatible, but also indicate that it is comparable to collagen biomaterials in terms of its persistence and long-term behavior in vivo.

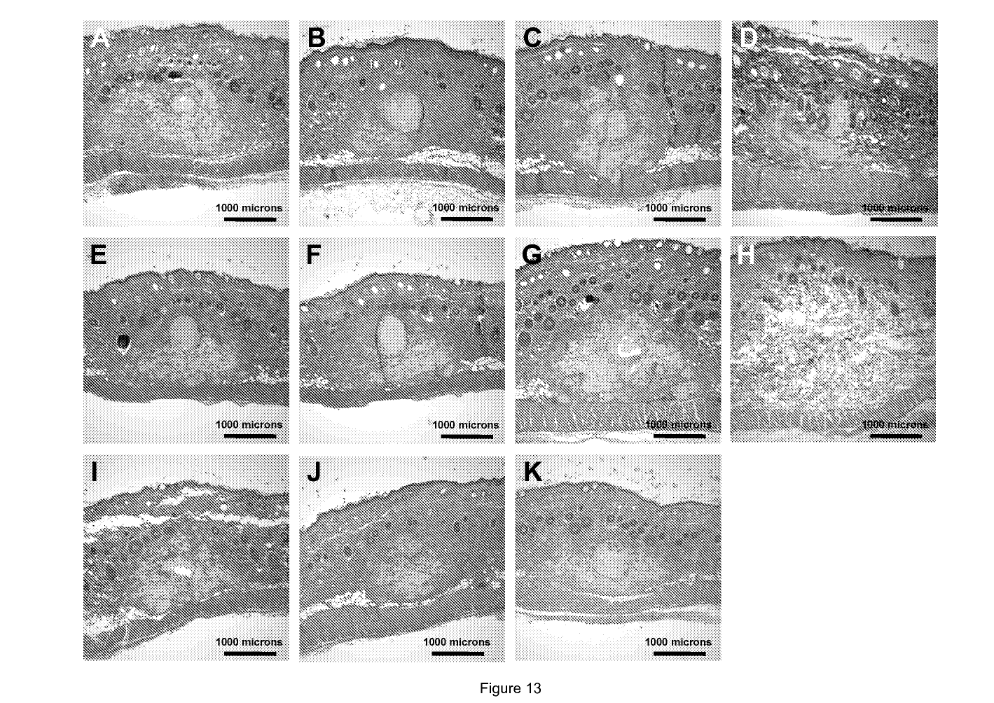

ZYPLAST™ exhibited no epidermal cysts, follicular atrophy, or sebaceous cell hyperplasia, though it did show small levels of fibrosis, inflammation and epidermal hyperplasia. Examination of histological sections showed residual silk gel material which stained in a mildly eosinophilic fashion and appeared as large lakes of material at a central location with smaller masses of material distributed more widely throughout the reticular dermis (FIG. 13). These smaller masses were typically surrounded by fibroblasts and macrophages with occasional multi-nucleated giant cells present. Eosinophils were located proximal to these smaller masses of implant as well. In general, host response to the silk fibroin gels was characterized as mildly fibrotic and included populations of fibroblasts, lymphocytes, macrophages, multi-nucleated giant cells and eosinophils. Little difference was evident between silk gel types except in terms of the extent of eosinophilia. Larger eosinophil populations were observed for 6% as compared to 4% silk gels and were also observed to increase with RGD concentration in the silk gel samples in both 4% and 6% groups. ZYPLAST™ exhibited strong eosinophilic staining and was distributed as large lakes in the reticular dermis with smaller masses throughout the area. Hypercellularity near the injection site was lessened in ZYPLAST™ samples when compared to silk gel. Fibroblasts, lymphocytes, macrophages, multi-nucleated giant cells and eosinophils were present with less tendency to localize at the implant periphery. HYLAFORM™ samples examined showed many very small masses of material throughout the reticular dermis. HYLAFORM™ exhibited no epidermal cysts, fibrosis, follicular atrophy, or sebaceous cell hyperplasia with extremely limited instances of inflammation and epidermal hyperplasia. There was no observable hypercellularity near the implanted material or other evidence of inflammation at the implant sites.

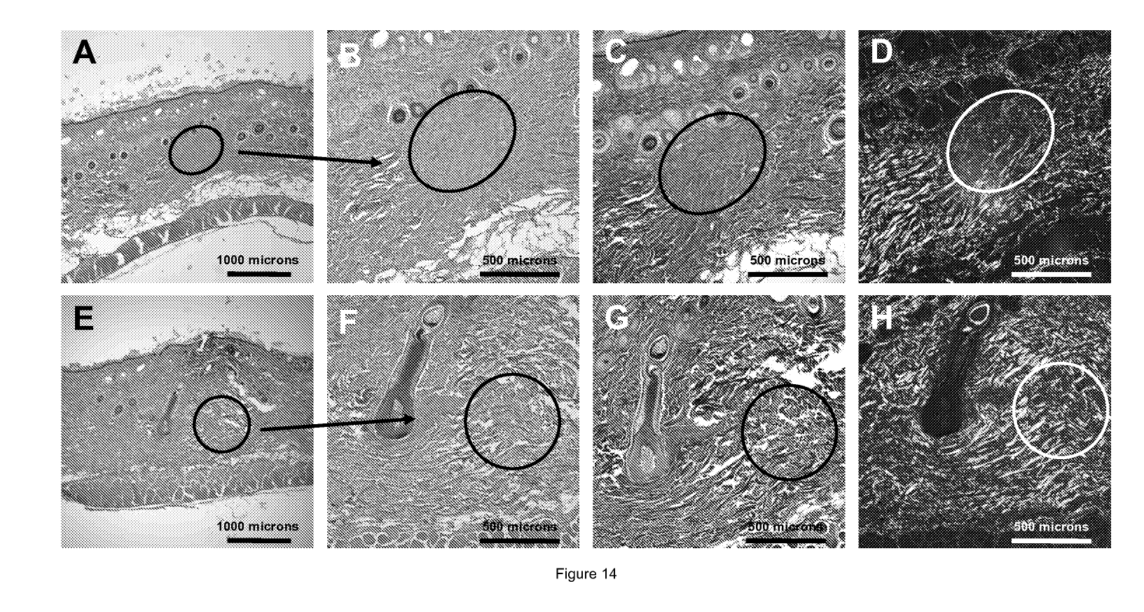

At day 92, histological evaluation of 4% silk fibroin hydrogel, 3:1 23RGD, 25% saline (4RH25) samples and ZYPLAST™ samples showed similar material persistence and host response (FIG. 14). Very little implant material remained visible in the dermis of the animals with no hypercellularity present at this time point, evidence of hyperplasia or cellular inflammation. The eosinophils found at day 28 in the ZYPLAST™ and silk gel samples were not observed at day 92. Of particular interest, 4RH25 also exhibited residual disruption to the reticular dermis in the form of an irregular collagen pattern near the implant material. The disorganization of the collagen was seen as a region of stained collagen seen to be devoid of the typical cross-hatch pattern of normal reticular dermis (FIG. 14C). This disorganization was confirmed when viewing the histological sections under polarized light with the disorganized collagen appearing as an interruption in the birefringence associated with the surrounding reticular dermis (FIG. 14D).

### Example 11

### Enhanced Injectable Gel Formulation

Silk hydrogels were prepared as described above in Examples 1-4. Once processed, the gels were sized into coarse or fine particles using a sieving step (Table 6). Gel materials were pressed through a 316SS stainless steel wire cloth sieve with a stainless steel spatula and into clean polystyrene Petri dishes. Sieves with gap sizes of 711 μm×711 μm, 295 μm×295 μm, 104 μm×104 μm and 74 μm×74 μm were used. After passing through the 74 μm×74 μm gap sieve, the material was considered processed to a “coarse” state. Samples to be processed to a “fine” state were further forced through a 43 μm×43 μm sieve in the same fashion. This sieving was conducted four separate times for each sample type, each sieving using an approximate quantity of 0.5 mL of gel material.

TABLE 6
Particle sizing

| Nominal | 23RGD | | |
|---|---|---|---|
| Silk Mass | Molar Ratio | | Group |
| Percentage | with Silk | Fineness | Name |
| 2% | 1:1 | Fine | 2RF |
| 4% | 0 | | 4F |
| | 1:1 | Coarse | 4RC |
| | | Fine | 4RF |
| | 3:1 | | RHRF |
| | 10:1 | | 4VHRF |
| 8% | 1:1 | Coarse | 8RC |
| | | Fine | 8RF |

Samples of silk gel material (N=4 of each type) were evaluated under light microscopy. Briefly, a 100 mg portion of silk gel or control device was massed using an SI-215 Summit series balance. This material was loaded into the open back end of a 3 mL syringe using a stainless steel spatula. The plunger was replaced in the syringe, an 18 g needle was attached to the end of the syringe and approximately 900 μL of ultra-pure water was drawn up. This mixture of water and silk gel was mixed through gentle shaking. After mixing to suspend evenly, a sample of approximately 30 μL of dilute silk gel was placed on a 75 mm×25 mm single frosted, pre-cleaned micro slide and covered with a 22 mm×40 mm premium cover glass (Corning Inc., Corning, N.Y.). This sample slide was then be imaged with a microscope. Sample slides were imaged using a System Microscope Model BX41 (Olympus, Melville, N.Y.) in conjunction with a Microscope PC MACROFIRE™ Model S99831 Camera (Optronics, Goleta, Calif.) and PICTUREFRAME™ 2.1 software (Optronics, Goleta, Calif.). Briefly, slides were scanned for clearly separated gel particles using the 4× objective lens and locations determined for a series of 3 representative images of the sample slide. Each of these locations was imaged after first switching the microscope objective lens to 10×. Micrograph image files were subjected to analysis with IMAGE-PRO® Plus 5.1 software (Media Cybernetics, Inc., Silver Spring, Md.). Image files were checked for particle size distribution, average particle size, average aspect ratio, maximum particle size, minimum particle size and standard particle size deviation. The data is presented in Table 7.

TABLE 7
Particle Comminution Data

| Group | Min to Max | Mean Object |
|---|---|---|
| Name | Object Area (μm²) | Area (μm²) |
| 2RF | 5.33 to 1.32 × 10⁴ | 52.43 ± 261.82 |
| 4F | 5.33 to 8.07 × 10³ | 27.82 ± 129.34 |
| 4RC | 5.33 to 8.52 × 10³ | 38.41 ± 196.67 |
| 4RF | 5.33 to 5.29 × 10³ | 34.12 ± 135.31 |
| 4HRF | 5.33 to 7.51 × 10³ | 40.62 ± 166.61 |
| 4VHRF | 5.33 to 3.14 × 10³ | 35.4 ± 105.43 |
| 8RC | 5.33 to 8.04 × 10³ | 46.57 ± 225.43 |
| 8RF | 5.33 to 2.85 × 10³ | 35.26 ± 129.63 |
| ZYPLAST ™ | 5.33 to 1.95 × 10³ | 22.08 ± 41.71 |

Examination of the particles under light microscopy revealed some clumped gel particles which were removed from particle sizing data manually. Particle sizes ranged from 5.3 to 1.3×10⁴ μm², comparable in range to commercially available ZYPLAST™ which ranged from 5.3 to 1.95×10³ μm². The data also revealed mean particle sizes ranging from 27.8 μm² to 52.4 μm², again, comparable to ZYPLAST™ with a mean particle size of 22.1 μm². These data illustrate that silk gel may be successfully comminuted to small and functionally useful particle sizes in a fashion similar to presently utilized injectable gel materials. The basic forced-sieving method can be replaced with other methods for scale-up purposes.

After comminution and blending, samples of silk gel emulsions were subjected to extrusion force testing. Gel materials prepared as described in Examples 1-4 were blended with appropriate ratios of saline in order to evaluate injection (extrusion) force profiles relative to a control material, ZYPLAST™ (Table 8). This was accomplished by massing 5 g of gel material in a large weighing boat using an SI-215 balance (Denver Instrument, Denver, Colo.). An appropriate quantity of saline will be added to constitute the correct volume percentage making the assumption that both the gel material and saline have a density of 1 g/mL. This material was then blended to an even consistency using a stainless steel spatula and loaded into the back end of a 10 mL syringe with an 18 g needle attached for subsequent use.

TABLE 8
Silk Gel Injection Force Profile Generation

| Nominal | 23RGD Molar Ratio | | Saline | Group |
|---|---|---|---|---|
| Silk Mass | with Silk | Fineness | Content | Name |
| 2% | 1:1 | Fine | 25% | 2RF25 |
| 4% | 0 | | 25% | 4F25 |
| | 1:1 | Coarse | 25% | 4RC25 |
| | | Fine | 0% | 4RFO |
| | | | 25% | 4RF25 |
| | | | 50% | 4RF50 |
| | 3:1 | | 25% | 4HRF25 |
| | 10:1 | | 25% | 4VHRF25 |
| 8% | | Fine | 25% | 6RF25 |

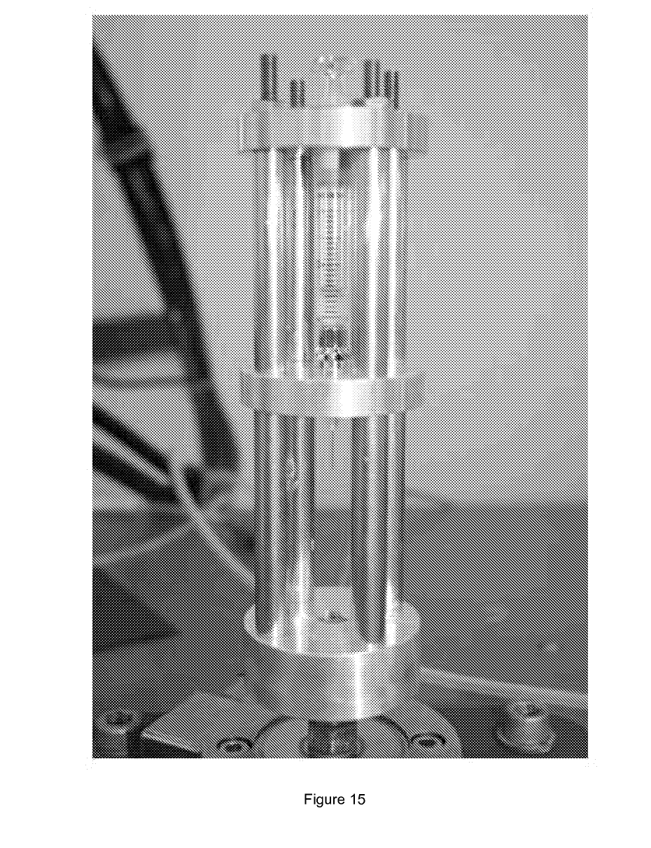

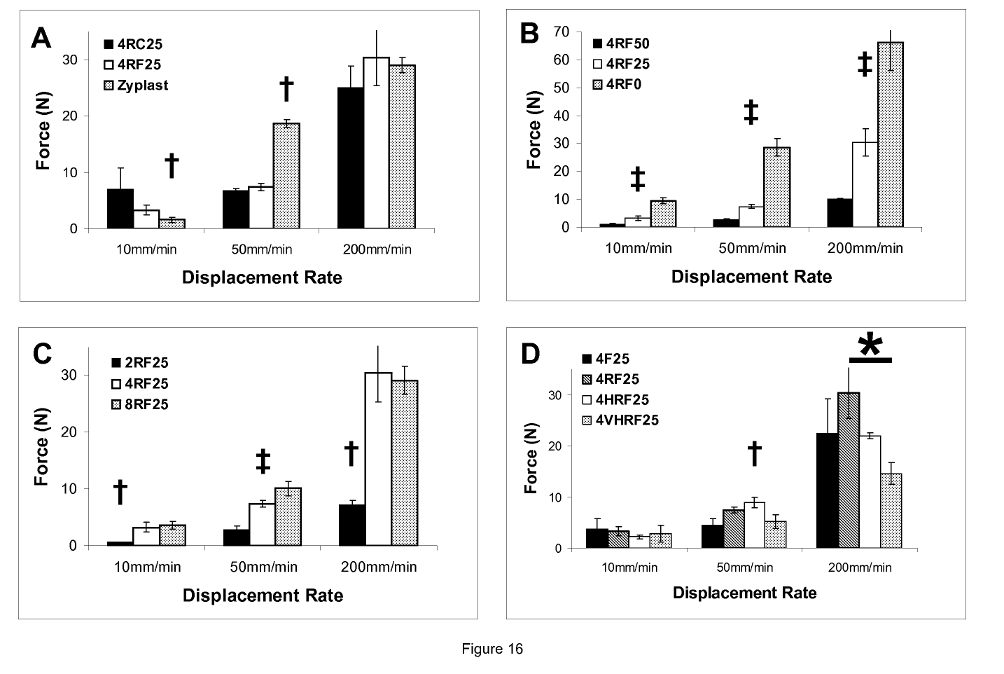

These samples were tested using an Instron 8511 (Instron Corp., Canton, Mass.) in conjunction with Series IX software and a custom-designed aluminum frame attached to a 100 N load cell (FIG. 15). For the material testing, 1 mL of the sample material of interest was loaded into a 1 mL gas-tight glass syringe. The sample syringe was mounted in the custom-designed aluminum frame mounted on the Instron unit and the material extruded. The sample was then checked for the force required to extrude the gel at each of 3 strain rates, 10 mm/minute, 50 mm/minute, and 200 mm/minute with total actuator displacement set at 7 mm. A series of four tests were run on each material type at each piston displacement rate. Load-displacement data was collected at a frequency of 100 Hz and are presented as the mean±the standard deviation of the 4 average extrusion forces experienced of each gel type at each strain rate. The average extrusion force was defined as the average load measured in the plateau region of the load-displacement curve resultant from each extrusion test. The data were reported as the average amount of force required for extrusion of the sample material and are compiled in Table 9 and FIG. 16.

TABLE 9
Average Force (N) to Extrude Silk Gel from 30 g Needle

| | Plunger Displacement Rate | | | | | |
|---|---|---|---|---|---|---|
| | 10 mm/min | | 50 mm/min | | 200 mm/min | |
| Group Name | Ave | Stdev | Ave | Stdev | Ave | Stdev |
| 2RF25 | 0.6 | 0.0 | 2.9 | 0.6 | 7.3 | 0.7 |
| 4RF25 | 3.7 | 2.0 | 4.5 | 1.3 | 22.4 | 6.7 |
| 4RD25 | 7.1 | 3.7 | 6.7 | 0.5 | 25.1 | 3.9 |
| 4RF0 | 9.5 | 1.0 | 28.5 | 3.1 | 66.2 | 10.0 |
| 4RF26 | 3.2 | 0.9 | 7.4 | 0.6 | 30.4 | 5.0 |
| 4RF50 | 1.2 | 0.2 | 2.7 | 0.1 | 10.1 | 0.3 |
| 4HRF25 | 2.2 | 0.4 | 8.9 | 1.0 | 22.0 | 0.6 |
| 4VHRF25 | 2.8 | 1.6 | 5.2 | 1.4 | 14.6 | 2.1 |
| 8RF25 | 3.6 | 0.7 | 10.1 | 1.3 | 29.2 | 2.4 |
| ZYPLAST ™ | 1.6 | 0.5 | 18.7 | 0.7 | 29.1 | 1.4 |

A comparison between milling techniques revealed that there were no significant differences between 4RC25 and 4RF25, having average extrusion forces of 7.1±3.7 N and 3.2±0.9 N at 10 mm/min, 6.7±0.5 N and 7.4±0.6 N at 50 mm/min, and 25.1±3.9 N and 30.4±5.0 N at 200 mm/min respectively (Table 6, FIG. 16A). Both formulations differed significantly (p≦0.05) from ZYPLAST™ at strain rates of 10 and 50 mm/min, which had extrusion forces of 1.6±0.5 N, 18.7±0.7 N, and 29.1±1.4 N at 10, 50, and 200 mm/min strain rates.

Data regarding the extrudability of silk gel formulations clearly illustrated that the addition of saline as a carrier fluid to the comminuted silk particles offers an improved degree of extrudability, substantially reducing the force necessary to extrude silk gel at all strain rates. Adding increasing concentrations of saline to the comminuted silk gels significantly decreased the extrusion force required for silk gels at each strain rate, with gels again exhibiting shear-thickening behavior (Table 9, FIG. 16B). At all strain rates, 4RFO required significantly more force to extrude than 4RF25, which in turn required significantly more than 4RF50. At a strain rate of 10 mm/min, 4R0, 4R10, and 4R25 showed a significant decrease (p≦0.05) in extrusion force with increasing PBS concentration, having average forces of 9.5±3.1 N, 6.1±0.5 N, and 4.7±0.7 N respectively (Table 9). At 50 mm/min, these relationships were more pronounced with average extrusion forces of 14.0±0.9 N, 5.4±0.7 N, and 3.9±0.2 N respectively and all differed significantly (Table 6, FIG. 16). At 200 mm/min, the trend remained as average extrusion forces were 26.4±4.5 N, 10.6±1.6 N, and 6.4±0.5 N respectively with 0% PBS differing significantly from the other two groups. Samples of 6R25 had an average extrusion force of 29.3±4.8 N at 10 mm/min, significantly higher than 4R25 (Table 9). At 50 mm/min and 200 mm/min, the force to extrude the 6R25 was greater than 80 N, causing the test to abort in order prevent damage to the load cell.

The data also illustrate that use of very low concentrations of silk may improve the extrudability of gel relative to higher concentrations as in the case of 2RF25 as compared to 4RF25 and 8RF25. Increasing the concentration of silk in the comminuted silk gels increased the extrusion force required for silk gels at each strain rate, with significant increases between 2RF25 and both 4RF25 and 8RF25 at 10 mm/min and 200 mm/min (Table 9, FIG. 16C). All groups differed significantly at the 50 mm/min strain rate and gels continued to exhibit shear-thickening behavior, seen in the increased extrusion forces associated with increased strain rates. At 10 mm/min 2RF25 and 8RF25 required 0.6±0.0 N and 3.6±0.7 N respectively, at 50 mm/min they required 2.9±0.6 N and 10.1±1.3 N, and at 200 mm/min 7.3±0.6 N and 29.2±2.4 N.

The data further indicated that use of 23RGD to enhance the silk gel material did not appreciably impact the force necessary to extrude silk gel formulations. Adding increasing concentrations of RGD did not have a consistent effect upon the extrusion force necessary for the gel materials (Table 9, FIG. 16D). At a 10 mm/min strain rate there were no significant differences between 4F25 at 3.7±2.0 N, 4R25, 4HR25 at 2.2±0.4 N, and 4VHR25 at 2.8±1.6 N. At a 50 mm/min strain rate 4HR25 was significantly higher than all other extrusion forces at 8.9±1.0 N as compared to 4F25 at 4.5±1.3 N, 4R25, and 4VHR25 at 5.2±1.4N. At a 200 mm/min strain rate 4HR25 at 22.0±0.6 N was significantly higher than only 4VHR25 at 14.6±2.1 N as compared to 4F25 at 22.4±6.7 N and 4R25.

Lastly, the data showed that silk gels blended with saline had very similar extrudability to ZYPLAST™, a material already proven to be readily handled as an injectable material. Based upon this data it is believed that through careful manipulation of the carrier species associated with the silk gel, modulation of silk concentration, and control of particle size, silk gel materials may be made to behave as a readily injectable material.

These results indicate that silk gels may be comminuted to a particle range of about 25-50 μm² in cross-sectional area. Silk gels may be comminuted to a size similar to ZYPLAST™. Silk gel particle size can be decreased by increasing silk concentration or by changing the method of comminution. Increasing concentrations of RGD did not develop a clear trend in silk particle size. Silk gels may be extruded at a relevant strain rate of 50 mm/min at a force comparable to or less than ZYPLAST™. Silk gel extrusion force may be decreased by adding increased quantities of saline carrier or decreased concentrations of silk in the original gel. Changes of comminution method attempted in this study did not substantially affect the amount of force necessary for silk extrusion. Increasing concentrations of RGD did not develop a clear trend in silk gel extrusion force.

### Example 12

### Silk Gel Precipitates

The silk gel precipitate materials outlined in Table 10 were generated for analysis. Silk solution of the specified concentration was generated using the stock solution of 8% (w/v) aqueous silk and diluting with purified water (Milli-Q purified). 23RGD/ethanol accelerant was prepared by generating a solution of ethanol and purified water, then dissolving the specified 23RGD quantity by vortexing. Silk precipitates were generated by directly adding the specified volume of accelerant solution to that of silk solution in 50 mL centrifuge tubes, shaking once to mix and allowing the mixture to stand for 5 additional seconds before adding about 45 mL purified water to halt the gelation process. This material stood for 24 hours under ambient conditions and was then strained through stainless steel cloth with 150 μm×150 μm pores to recover precipitates. These precipitates were rinsed twice for 24 hours in 50 mL of purified (Milli-Q) water at room conditions, strained a final time and used for evaluation.

TABLE 10
Silk Gel Precipitate Types Generated

| | Initial Silk Solution | | 23RGD/ethanol Accelerant | | | Final Precipitate | |
|---|---|---|---|---|---|---|---|
| | Silk | Silk | | 23RGD | Accelerant | Final Silk | RGD:Silk |
| Group | Concentration | Solution | Ethanol | Concentration | Solution | Concentration | Molar |
| Name | (mg/mL) | Volume (mL) | Concentration (%) | (mg/mL) | Volume (mL) | (mg/mL) | Ratio |
| BASE | 80 | 1 | 90 | 2.45 | 1 | 40 | 5.0 |
| RHI | 80 | 1 | 90 | 4.90 | 1 | 40 | 10.0 |
| RVLO | 80 | 1 | 90 | 0.49 | 1 | 40 | 1.0 |
| RLO | 80 | 1 | 90 | 1.47 | 1 | 40 | 3.0 |
| SCLO | 80 | 1 | 90 | 2.45 | 1 | 30 | 6.7 |
| SCVLO | 80 | 1 | 90 | 2.45 | 1 | 20 | 10.0 |
| ECLO | 80 | 1 | 80 | 2.45 | 1 | 40 | 5.0 |
| ECVLO | 80 | 1 | 70 | 2.45 | 1 | 40 | 5.0 |
| AVHI | 80 | 0.67 | 90 | 2.45 | 1.33 | 27 | 10.0 |
| AVLO | 80 | 1.33 | 90 | 2.45 | 0.67 | 53 | 2.5 |

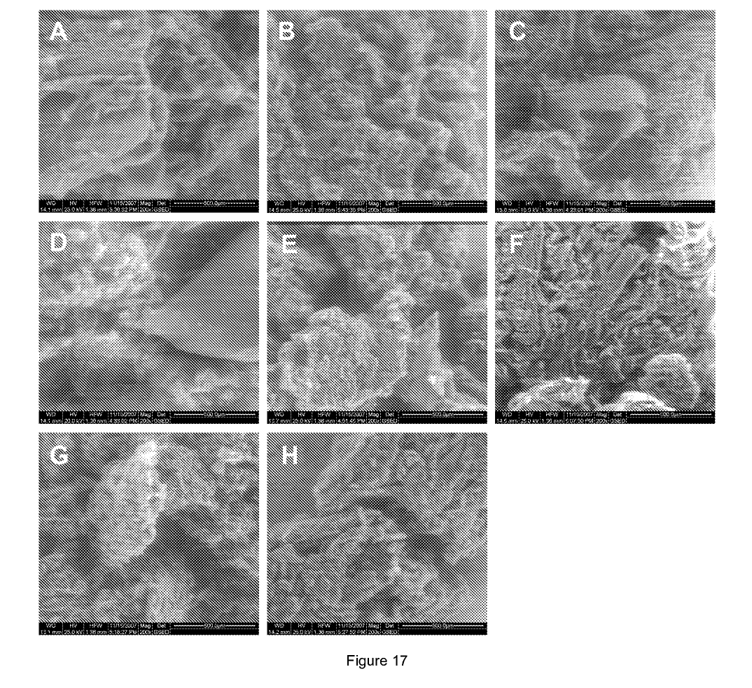

Samples of gel were examined under low-vacuum conditions (−1 Torr) on a Quanta 200 (FEI Co., Hillsboro, Oreg.) environmental scanning electron microscope with images collected at magnifications of 200×. Representative images were taken to illustrate surface topography characteristics of silk precipitate samples (FIG. 17). All silk precipitate types appeared similar under ESEM analysis. Each sample exhibited a mixture of both granular and filamentous regions with occasional appearance of large, contiguous masses of smooth material.

### Example 13

### Silk Gel Precipitate Massing

Silk precipitate samples, as described in Example 12, were isolated after rinsing by straining through stainless steel wire cloth with a pore size of 104 μm×104 μm and gently blotted with a clean, lint-free wipe. Samples were massed to the nearest 0.01 mg using an S-215 balance (Denver Instrument, Denver, Colo.). These samples were frozen to −80° C. for 24 hours and placed into a Labconco lyophilizer unit (Labconco Corp., Kansas City, Mo.) for 96 hours to remove all water content. The precipitate residual solids were massed again and the dry mass fraction in the samples determined. One-Way analysis of variance (ANOVA) was used to test for significant differences caused by changing silk concentration, 23RGD concentration and accelerant volume. A Student's t-test was used to test the significance of differences observed.

Increasing silk fibroin concentration increased precipitate dry mass with Increasing the percentage of ethanol in the accelerant solution also increased dry mass of the precipitates with ECVLO produced only trace quantities of precipitate (visible, but not recoverable in measurable quantities).

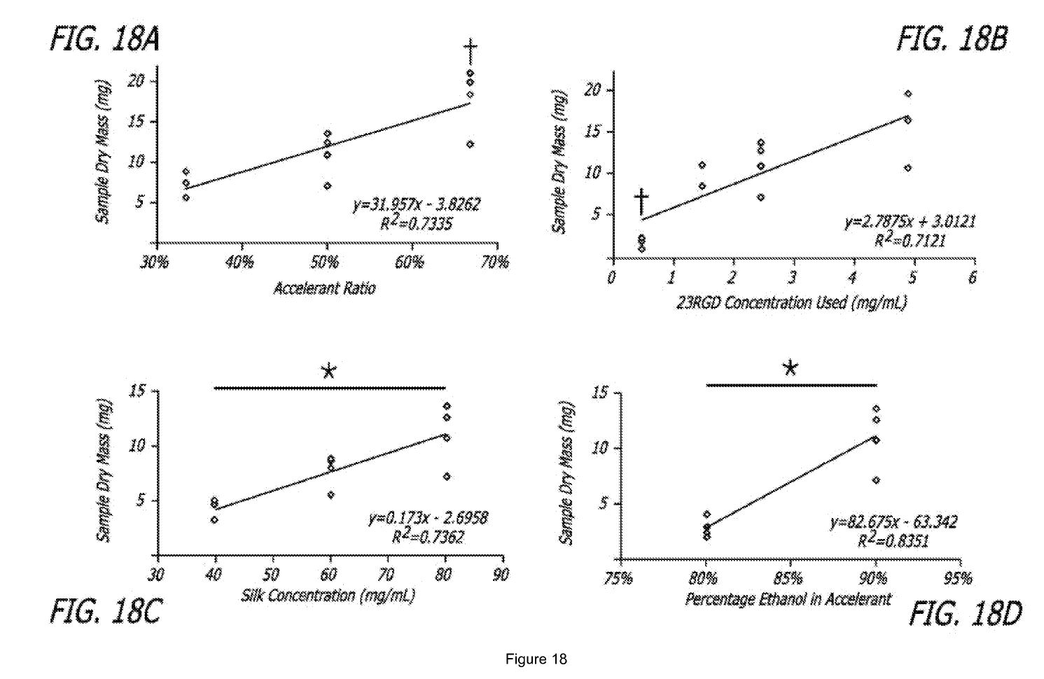

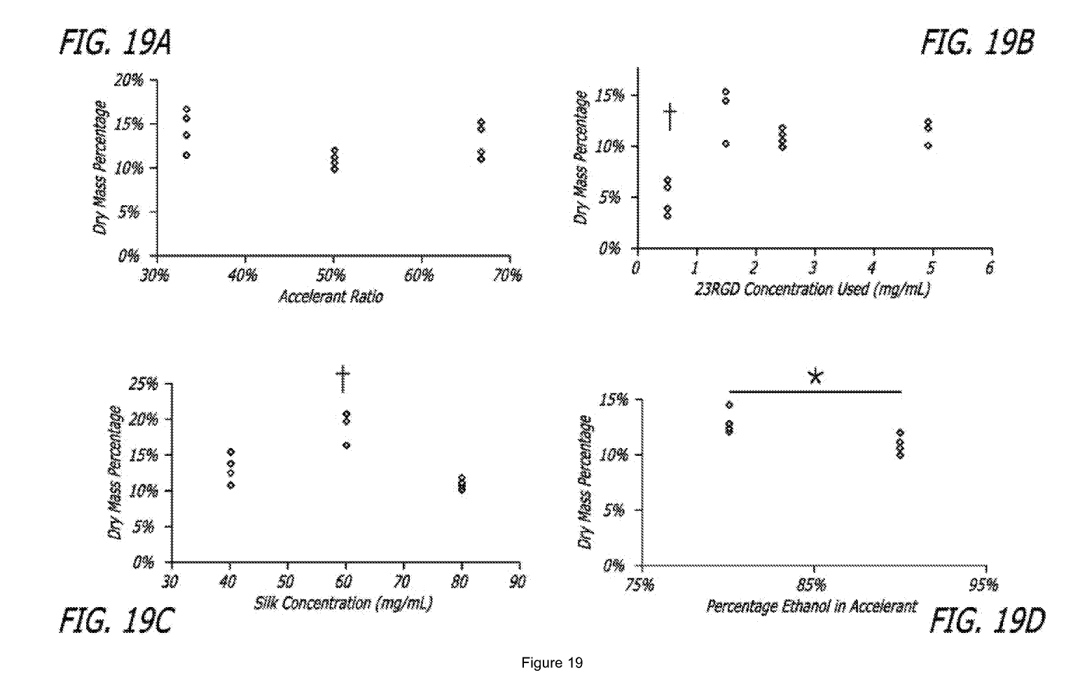

Increasing accelerant volume significantly increased precipitate dry mass as AVHI was significantly greater than both AVLO and BASE (p≦0.05, FIG. 18A). For example, AVHI (18.02±3.9 mg) was significantly greater than both AVLO (7.37±1.33 mg) and BASE (11.07±2.86 mg). Increasing concentrations of 23RGD in the accelerant also increased the dry mass of precipitate with BASE and RHI both significantly higher than RVLO at (FIG. 18B). Fore example, BASE at 11.07±2.86 mg, RHI at 15.61±3.62 mg, and RMED at 10.2±1.42 mg were all significantly higher than RLO at 1.9±0.6 mg. Increasing silk fibroin concentration increased precipitate dry mass with BASE being greater than SCLO and significantly greater than SCVLO (FIG. 18C). For example, BASE was greater than SCLO at 7.84±1.49 mg and significantly greater than SCVLO at 4.15±1.0 mg. Increasing the percentage of ethanol in the accelerant solution also increased dry mass of the precipitates with BASE producing significantly more than ECLO (FIG. 18D). For example, BASE produced significantly more than ECLO at 2.8±0.91 mg. ECVLO produced only trace quantities of precipitate. These results indicate that greater concentrations of reactants (i.e., accelerant solution, RGD, silk and ethanol) all increased the quantity of precipitant resultant.

The percent water in silk precipitates was determined as the percentage of mass lost after silk precipitates of each formulation types were subjected to a lyophilization step. Increasing the volumetric fraction of accelerant added to make silk precipitates did not significantly (p≦0.05) affect the dry mass fraction of the resultant precipitates (FIG. 19A). For example, AVLO at (85.57±2.32%, BASE at 88.99±0.8%, and AVHI was 86.83±1.95%. Increasing concentrations of 23RGD in the accelerant showed a significant increase in dry mass percentage with RVLO significantly less than RLO, RHI, and BASE (FIG. 19B). For example, RLO at 95.01±1.76% retained significantly more water than RMED at 86.52±2.67%, RHI at 88.39±0.98%, and BASE. Increasing concentrations of silk fibroin did not result in a clear trend although SCLO was significantly greater than both SCVLO and BASE (FIG. 19C). For example, SCLO at 80.77±1.97% was significantly less than both SCVLO at 86.94±1.98% and BASE. Increasing the percentage of ethanol in the accelerant solution significantly decreased the dry mass percentage with ECLO compared to BASE (FIG. 19D). For example, ECLO at 86.97±1.16% compared to BASE. In summary, greater concentrations of reactants (i.e., accelerant solution, 23RGD, silk and ethanol) increased the quantity of resultant precipitate. It is also of interest that there were significant differences between the dry mass fractions of BASE and both RVLO and ECLO, possibly indicating different protein structures. These differing protein structures might be more hydrophobic than BASE in the case of ECLO and more hydrophilic in the case of RVLO. These properties can affect the stability of gels in an in vivo environment with more hydrophilic materials being more readily bioresorbed by the host while more hydrophobic materials prove more resistant.

In examining the percent of water in the precipitates it is of particular interest that there were significant differences between BASE and both RLO and ECLO. This may result from structural motifs different than other precipitate types generated by RLO and ECLO. With respect to ECLO, it has a greater proportion of β-sheet structure than BASE and would be expected to entrain less water. However, the difference observed between RLO and base is difficult to explain. RLO has a greater extent of β-sheet structure with less α-helix and random coil motifs than BASE, yet it entrains a greater quantity of water. In fact, this same trend is seen when comparing RLO to RMED, BASE, and RHI. The situation is further confounded in examining the relationship between the initial secondary structures of RMED, BASE and RHI, as all initially exhibit greater quantities of α-helix and random coil than RLO, yet all entrain significantly less water. SCLO samples also had a significantly higher dry mass percentage as compared to BASE and SCVLO sample with no clear trend or reason for this occurrence. These data indicate that there may be a structural difference in these precipitates not apparent in the secondary structure of the materials which is affecting the manner in which the precipitates associate with water. It may be the case that the RGD bound to these precipitates has altered in some fashion the manner in which the silk molecules are presented to water, enhancing their ability to associate with it.

### Example 14

### Gel Precipitate FTIR Spectrum Capture

Gel precipitates of each type, as described in Example 12, were analyzed by attenuated total reflectance Fourier-transform infrared (ATR-FTIR) spectroscopy using a Bruker Equinox 55 spectrophotometer (Bruker Optics, Inc., Billerica, Mass.) coupled with a Pike MIRACLE™ germanium crystal (PIKE Technologies, Madison, Wis.). Sample ATR signal spectra were obtained by performing a 128-scan integration. Resolution was set to 4 cm⁻¹ with a 1 cm⁻¹ interval from a range of 4000 to 400 cm⁻¹. FTIR spectra of pure water were also collected and subtracted manually from the gel spectra to remove confounding water signal at a ratio conducive to flattening the region between 1800 cm⁻¹ and 1700 cm⁻¹ on the spectrum. After subtraction, the Amide I bands (1700-1600 cm⁻¹) of representative spectra were evaluated against characteristic peaks commonly accepted to be associated with secondary protein structures.

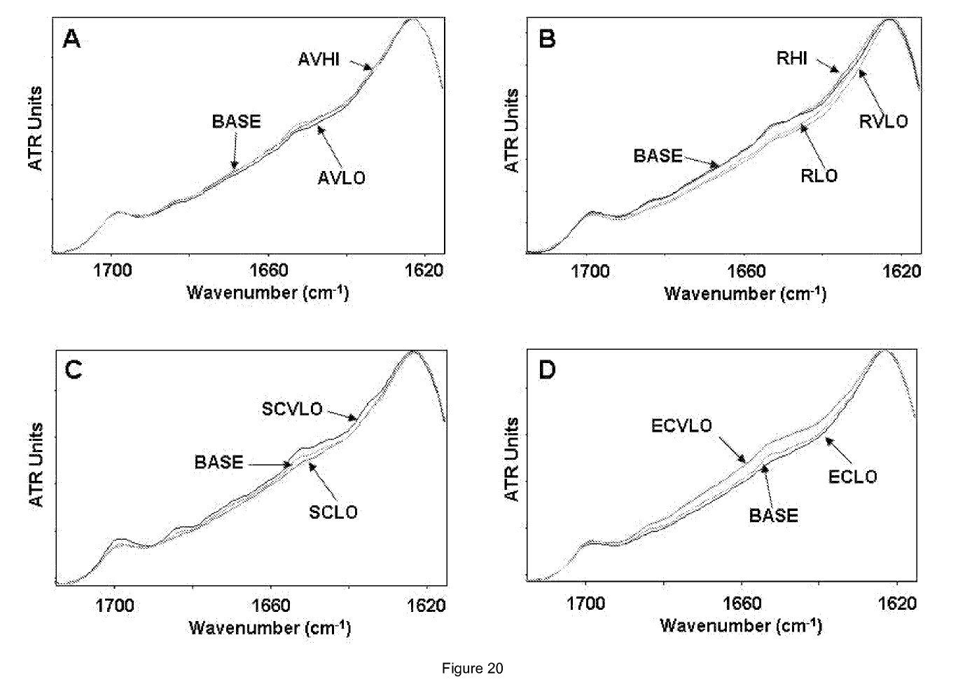

Examination of the silk precipitates under FTIR revealed that increasing the volumetric ratio of accelerant added to the silk solution had little effect on their protein secondary structure (FIG. 20A). AVLO, BASE, and AVHI all exhibited similar characteristics with characteristic peaks around 1624 cm and a toe region at 1698 cm⁻¹ indicating a predominance of β-sheet and β-turn structure respectively. Each sample also exhibited additional structures at 1677 cm⁻¹, 1663 cm⁻¹, 1654 cm⁻¹ and 1645 cm⁻¹ denoting additional interspersed β-sheet, β-turn, α-helical and random coil conformations respectively. Increasing concentrations of 23RGD in the accelerant decreased β-sheet stability indicated by a peak shift from ˜1621 cm⁻¹ in RVLO to ˜1624 cm⁻¹ in RLO (FIG. 20B). Further increasing the concentration of 23RGD in BASE and RHI caused this weakened β-sheet again accompanied by an increase in higher signal values in the 1654 cm⁻¹ and 1645 cm⁻¹ ranges, indicating increased random coil and α-helical constituents. Otherwise, RVLO, RLO, BASE, and RHI revealed similar structures with dominant peaks in the 1620 cm⁻¹ range and a toe region at 1698 cm⁻¹ with additional structures at 1654 cm⁻¹ and 1645 cm⁻¹. Increasing concentrations of silk fibroin had little perceptible effect on protein secondary structure (FIG. 20C). The spectra for SCVLO, SCLO, and BASE each exhibited similar peaks around 1624 cm⁻¹ with toe regions at 1698 cm⁻¹ indicating a predominant β-sheet structure with additional α-helical and random coil conformations interspersed. Increasing the percentage of ethanol in the accelerant solution resulted in less α-helical and random coil conformations indicated by a decrease in the signal between 1670 cm⁻¹ and 1630 cm⁻¹ in both ECLO and BASE samples relative to ECVLO (FIG. 20D). This decrease in α-helical and random coil is accompanied by an increase in β-sheet structure.

Substantial similarity existed between all groups except for RVLO and ECVLO, which each differ from BASE formulation. Each of these material types exhibited a different secondary structure from both each other and from BASE, reinforcing the trend observed previously in the percent dry mass of the precipitates. Higher concentrations of 23RGD yielded less organized β-sheet structures and lower concentrations of ethanol yielded greater quantities of α-helix and random coil motifs. It is possible that used in conjunction with one another, these two phenomena could be adjusted to develop silk structures resulting from silk solutions in any of a variety of different protein conformations. These conformations could, in turn, be tailored based upon the desired ultimate bulk properties of the silk material. It is expected that higher β-sheet components might provide the gel with greater resistance to bioresorption and compressive loading, while at the same time, making the material more rigid.

### Example 15

### Congo Red Staining of Gel Precipitates

Silk precipitate samples were stained with 100 μM Congo red in purified water. Silk precipitate samples weighing 5-10 mg were vortexed with 500 μL of this solution for 15 seconds, allowed to stand at room temperature (˜20-24° C.) for 10 minutes, then centrifuged at 16,000 g (RCF) for 10 minutes. The supernatant was discarded and the pellet re-suspended by vortexing for 30 seconds in 1 mL of purified water. The process of soaking, centrifugation, aspirating and rinsing was repeated 3 times. The final pellet was removed, smeared on a glass microscope slide, and imaged under white and polarized light using a Microscope PC MACROFIRE™ Model S99831 Camera (Optronics, Goleta, Calif.) and PICTUREFRAME™ 2.1 software (Optronics, Goleta, Calif.) and a System Microscope Model BX41 (Olympus, Melville, N.Y.).

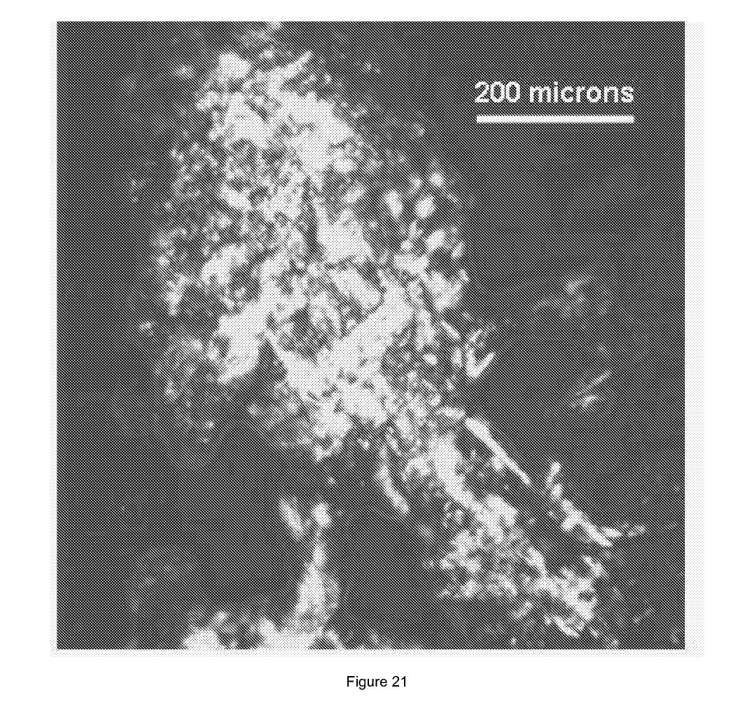

None of the silk precipitate types exhibited the emerald luminescence typically associated with amyloid fibrillar structures (FIG. 21). All precipitate types did exhibit bright white luminescence, indicative of a robust crystalline structure. The extent of this brightness does not appear to vary substantially by formulation, but only by sample quantity on the slide. Based on these results, it is unlikely that any of these precipitate types is amyloid in nature, a positive sign, as amyloid fibrils are associated with a number of negative pathologies in humans.

### Example 16

### 23RGD Quantification in Gel Precipitates by HPLC

The amount of 23RGD bound to silk precipitates was quantified by analyzing lyophilized samples. The 23RGD was removed by incubating the samples for 4 hours in a dissolving buffer, then centrifuging on an Eppendorf 5415C (Eppendorf North America Inc., Westbury, N.Y.) at 16,000 g (RCF) for 30 minutes and the supernatant collected. This supernatant was then centrifuged in identical fashion and the final supernatant collected for HPLC analysis using a PerkinElmer Series 200 (PerkinElmer, Waltham, Mass.). The 23RGD peak areas from each curve were compared against a standard curve. 1-Way ANOVA was used to test for significant differences caused by changing silk concentration, 23RGD concentration, and accelerant volume. A Student's t-test was used to test the significance of differences observed.

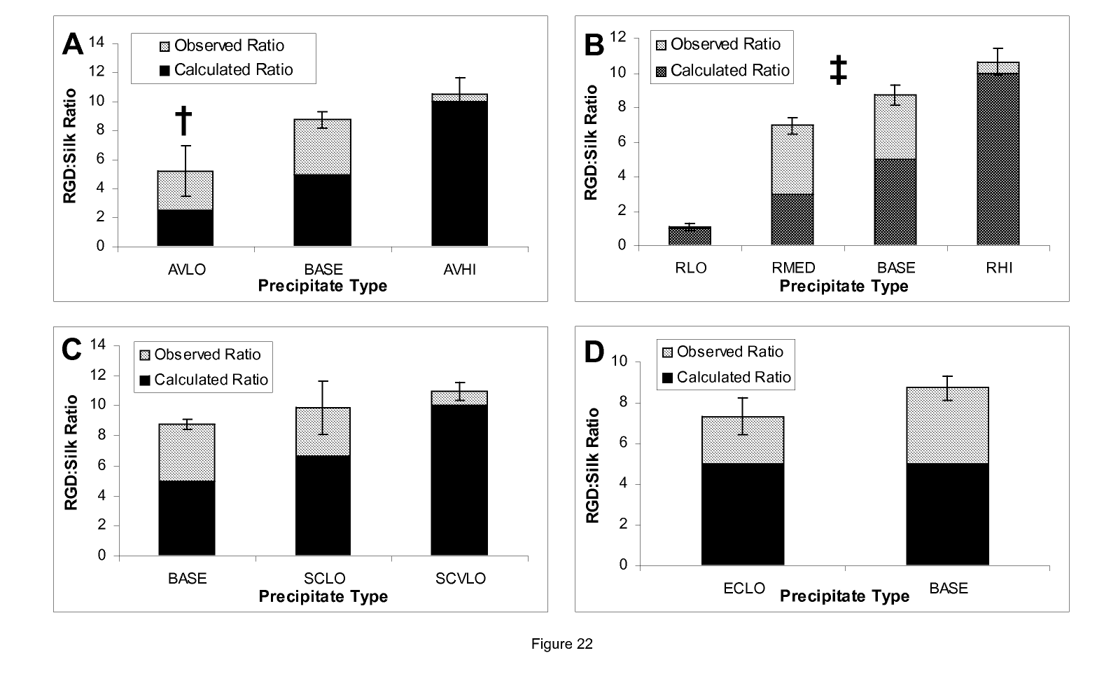

Increasing the quantity of 23RGD/ethanol accelerant added resulted in a significant increase (p≦0.05) in 23RGD:silk ratio for both BASE and AVHI as compared to AVLO (FIG. 22A). For example, BASE at 8.7±0.6 and AVHI at 10.5±1.2 were significantly increased as compared to AVLO at 5.2±1.8. Increasing the quantity of 23RGD in the accelerant solution resulted in significant increases in 23RGD:silk ratio for each of RVLO, RLO, BASE, and RHI relative to each other (FIG. 22B). For example, RLO at 1.1±0.2, RMED at 6.95±0.49, BASE and RHI at 10.7±0.8 relative to each other. Changing the starting concentration of silk in solution prior to precipitation did not affect 23RGD:silk ratio as those in SCVLO, SCLO, and BASE did not differ significantly (FIG. 22C). For example, SCVLO at 11.0±0.4, SCLO at 9.9±1.8, and BASE did not differ significantly. Decreasing the ethanol content in the accelerant did not produce a significant effect as observed by comparing ECLO and BASE (FIG. 22D).

Reviewing this data in light of the precipitate dry massing data, none of the conditions explored resulted in isolation of silk (˜10-35% precipitated) nor 23RGD (˜5-30% precipitated) as limiting reagents in the reaction. Precipitate samples generated at a calculated 10:1 23RGD:silk ratio consistently generated a “correct” molecular binding ratio. In the case of AVHI, this runs contrary to the trend of bound 23RGD concentrations being approximately double the projected values as indicated by AVLO and BASE (about 5:1 and about 9:1, respectively). This might be explained by saturation of the silk with 23RGD in the case of 10:1 23RGD precipitates. This is further reinforced by the behavior of SCVLO and 0.6S 3R 10:1, both of which were produced using 2.45 mg/mL 23RGD in 90% ethanol as the AVHI was. Both materials projected to have greater than 10:1 ratios of bound 23RGD (20:1 and 13.4:1, respectively) based on the behavior of AVLO and BASE, but which both reached only about 10:1 ratios. RHI, generated using a 4.5 mg/mL 23RGD concentration in the accelerant which conceivably should have been high enough to induce the postulated dimeric 23RGD reached only the expected 23RGD ratio of about 10:1 not the postulated 20:1.

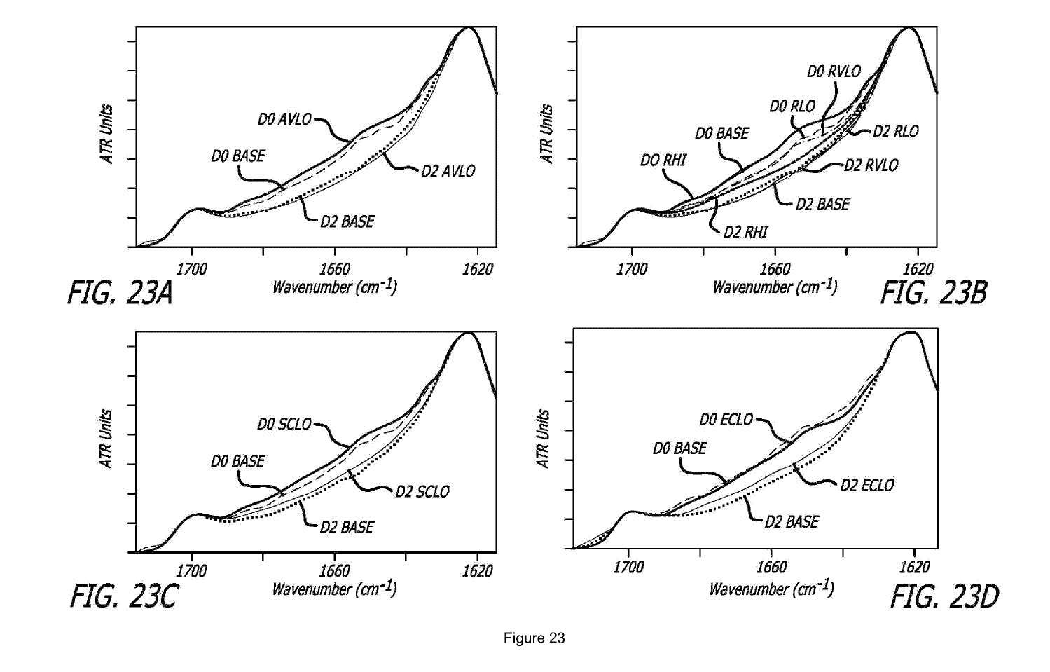

Few of the silk precipitates entrained a molar ratio similar to what was initially calculated (FIG. 23). Four groups, SCVLO, AVHI, RHI, and RLO contained ratios similar to their calculated values of RGD per mole of silk. The six remaining groups contained ratios substantially greater than their calculated values. In the cases of AVLO, BASE, RMED, and SCLO, the RGD quantities were about 2-fold greater than expected. This greater observed molar ratio may be indicative of the formation of a RGD bi-layer. It may be the case that either micelles or lamellar structures of RGD existed in the 90% ethanol solution prior to addition to the silk, upon contacting the aqueous phase, micellar stability was disrupted. As a result, a bi-layer of RGD was formed at the solution interface, where these molecules began to interact with the silk molecules. The RLO samples were made with a RGD concentration of 0.49 mg/mL in the accelerant, the lowest used in this study and potentially within the solubility range of RGD in 90% ethanol. RMED samples used 1.47 mg/mL and most other formulations were made with a RGD accelerant concentration of 2.45 mg/mL, above the RGD concentration at which dimerization became favorable in the solution. Further highlighting the possibility of RGD dimerizing in the ethanol solution is the behavior of ECLO precipitation. The RGD concentration remains 2.45 mg/mL as with BASE and AVLO but the water concentration in the accelerant is increased to 20% and results in a binding of about 1.5-fold the expected total of RGD. This may be due to a decreased driving force for RGD bi-layer formation at the solution interface caused by the lower ethanol content which, in turn, disrupts formation of micellar structures in the initial accelerant solution. It could also be explained by altered micellar structure, varying between a single peptide layer and a multi-lamellar structure depending upon the concentrations of water and ethanol in the accelerant phase.

Precipitate samples generated at a calculated 10:1 RGD:silk ratio consistently generated a “correct” molecular binding ratio. In the case of AVHI, this runs contrary to the trend of bound RGD concentrations being approximately double the projected values as indicated by AVLO and BASE (about 5:1 and about 9:1 respectively). This might be explained by saturation of the silk with RGD in the case of 10:1 RGD precipitates. This is further reinforced by the behavior of SCVLO and 0.6S 3R 10:1, both of which were produced using 2.45 mg/mL RGD in 90% ethanol as was AVHI. Both materials were projected to have greater than 10:1 ratios of bound RGD (20:1 and 13.4:1 respectively) based on the behavior of AVLO and BASE, but both reached only about 10:1 ratios. RHI, which conceivably should have been high enough to induce the postulated dimeric RGD, reached only the expected RGD ratio of about 10:1 not the postulated 20:1. This may be attributed to the mode of binding between the silk molecules and the RGD molecules. It is expected that RGD will bind through a hydrophobic association mechanism and despite the largely hydrophobic sequence of silk, it may be possible that there are approximately 5 sites which offer preferable RGD binding stability. This presumption stems from the apparent saturation at 10:1 RGD molecules per molecule of silk. Dependent upon the nature of RGD self-association at the solution boundary, it may be a case where single RGD molecules or RGD dimers bind to these sites.

There are a series of properties further indicating the possibility of a specific molecular assembly interaction between the silk and 23RGD accelerant. Conspicuously, that 23RGD does localize to the precipitates in a greater-than-calculated ratio but that it binds at intuitive concentrations which can be related quickly to the initially calculated molar ratios. The fact that this occurs without fully depleting either the 23RGD or the silk fibroin molecules is of further interest. The FTIR data also indicated that use of 0.49 mg/mL 23RGD in RVLO precipitates induced formation of distinctly different structures than use of 2.45 mg/mL in BASE or 4.9 mg/mL in RHI which appeared similar to each other. RMED precipitates generated with 1.47 mg/mL of 23RGD contained characteristics of both RVLO and BASE/RHI material spectra. FTIR indicated a different structure from a 2.45 mg/mL of 23RGD in 70% ethanol accelerant in the case of ECVLO. These outcomes were both reinforced in examining the percentage of dry mass from the resultant precipitates (though ECLO is used to illustrate the trend in 23RGD solubility in ethanol solution instead of ECVLO). Both assays indicate the formation of different precipitate structures based upon the extent of 23RGD saturation in the ethanol solution, conceivably resulting from dimeric 23RGD binding or monomeric 23RGD binding.

This phenomenon likely results from the amphiphilic nature of 23RGD and the varied chemistry of the solution phase between heavily ethanolic and heavily aqueous. It is possible that the hydrophilic ends of two 23RGD molecules associate in the 90% ethanol solution, exposing the two hydrophobic ends to solution. Addition of this accelerant solution with dimeric 23RGD causes rapid association of the exposed hydrophobic ends of the 23RGD with hydrophobic domains of the silk molecules, rapidly precipitating them. This process occurs until the 90% ethanol accelerant solution is sufficiently diluted with the aqueous silk solution to cause the dimeric assembly of the 23RGD molecules to no longer be favorable, as a result stopping precipitation. Based upon the apparent saturation at about 10 for 23RGD:silk ratio, there may also be a maximum of 5 binding sites for the 23RGD dimer per molecule of silk. This knowledge may be used to bind specific quantities of 23RGD to silk, while at the same time dictating silk gel structure and resultant behavior. Additionally, this method may also potentially be applied to other amphiphilic peptides of interest during their integration into a silk gel material.

These results indicate that silk precipitate quantity may be increased by increasing the quantity of any reactant in the RGDEEG system. Silk precipitates occurring during RGDEEG gelation are unlikely to be amyloid. Silk precipitate β-sheet structure may be increased by higher concentrations of ethanol accelerant or lower concentrations of RGD. RGD molecules may self-associate into micelles, lamellar structures, or dimers when placed into a strongly ethanolic solution, in turn, assembling with silk in a dimeric fashion during RGDEEG gelation. Silk molecules may become saturated with RGD once they have bound about 10 molecules. Silk precipitate structures may be altered by changing RGD concentrations added, though the extent and nature of these changes remains unclear, as they are not perceptible in material secondary structure. These altered structures may account for otherwise unexplained increased appearance of α-helix and random coil motifs at high RGD concentrations in precipitates. These altered structures may account for otherwise unexplained increased resistance to proteolytic bioresorption of α-helix and random coil motifs at high RGD concentrations in precipitates

### Example 17

### Enzymatic Bioresorption of Gel Precipitates

A single sample of precipitate types selected for distinctly different behaviors from BASE in the previously listed assays including RVLO, RLO, BASE, RHI, ECLO, 0.6S 3R 5:1 weighing approximately 60 mg were massed using an S-215 balance. These samples were placed in a solution of Protease Type XIV (Sigma P-5147) in PBS generated at a concentration of 0.3 mg/mL (activity was 4.5 U/mg) at a ratio of 1 mL of protease solution per 100 mg of silk precipitate. The gel and protease solution were incubated for 24 hours at 37° C. with no mechanical mixing. After 24 hours, the residual precipitate was isolated by straining through stainless steel cloth as before and the specimens analyzed by FTIR as described.

Accelerant quantity added did not substantially affect the bioresorption behavior of the materials as BASE, AVHI and AVLO all featured decreased levels of α-helix and random coil motifs (FIG. 23A). This decrease was slightly larger in the case of AVLO which also featured a peak shift from 1624 cm⁻¹ to 1622 cm⁻¹, indicating a more stable β-sheet structure. The 23RGD concentration did not appear to affect bioresorption behavior of the materials either as RVLO, RLO, BASE and RHI all showed decreased in α-helix and random coil motifs, though a greater portion of α-helix and random coil remained intact in RHI (FIG. 23B). However, a greater portion of α-helix and random coil remained intact in RHI at Day 2 relative to the other samples. Silk concentration did not substantially affect the bioresorption behavior of the materials as BASE and SCLO exhibited decreased levels of α-helix and random coil motifs and featured slight peak shifts from 1624 cm⁻¹ to 1623 cm⁻¹ (FIG. 23C).

Despite differences in initial structures, all precipitate types bioresorbed in a similar fashion with α-helix and random coil motifs degraded preferentially to β-sheet. Only increasing the concentration of 23RGD, as in the case of RHI, appeared to have any appreciable effect on the final secondary structure of the precipitates. This appears to be a case where there is simply more α-helix and random coil structure upon initial formation of these materials and they take more time to degrade to a similar extent of β-sheet structure as the other formulations. Use of this knowledge in conjunction with an ability to manipulate the secondary protein structures of these materials could lead to biomaterials with very specific lifetimes in vivo.

### Example 18

### Composition to Treat a Wound

This example illustrates how to topically apply a composition disclosed herein to treat a wound.

A woman has a cosmetic surgery performed on her facial features by a qualified plastic surgeon. After the surgery is completed, the physician topically applies an ointment comprising a composition disclosed herein. After being released by the physician, he prescribes the same ointment for the woman so she can apply this treatment twice a day to her wounds. Within three days, the surgical wounds are healing at a rate 50% faster than without the ointment.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to practice the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A drug delivery platform comprising:
a) a substantially sericin-depleted silk fibroin;
b) an amphiphilic peptide; and
c) a compound having the structure of formula I 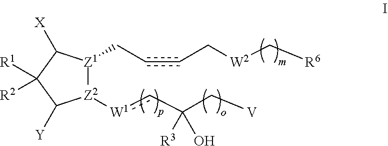
wherein each dashed line represents the presence or absence of a bond;
R¹, R² and R³ are each independently selected from H or C₁-C₆ alkyl;
R⁶ is CO₂H, CO₂R⁷, CON(R⁷)₂, CONHCH₂CH₂OH, CON(CH₂CH₂OH)₂, CH₂OR⁷, P(O)(OR⁷)₂, 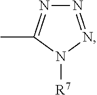
a pharmaceutically acceptable salt thereof, or a pharmaceutically acceptable amine salt thereof;
R⁷ is independently H, C₁-C₆alkyl or C₂-C₆alkenyl;
X and Y are each independently selected from H, OH, ═O, Cl, Br, I, or CF₃;
Z′ and Z² are each independently selected from CH or N;
W¹ and W² are each independently selected from CH, CH₂, aryl or substituted aryl, heteroaryl, substituted heteroaryl;
m is 0 to 4;
o is 0 to 6;
p is 0 or 1; and
V is C₁-C₆ alkyl, aryl, substituted aryl, heteroaryl, or substituted heteroaryl.

2. The drug delivery platform of claim 1, wherein the hydrogel comprises about 1% (w/v) to about 10% (w/v) of silk fibroin.

3. The drug delivery platform of claim 1, wherein the amphiphilic peptide comprising a RGD motif or a non-RGD integrin.

4. The drug delivery platform of claim 1, wherein the hydrogel comprises a molar ratio of 1:10 to 10:1 moles of the amphiphilic peptide per mole of the silk fibroin or a molar ratio of 3:1 moles of the amphiphilic peptide per mole of the silk fibroin.

5. The drug delivery platform of claim 1, wherein the hydrogel comprises silk fibroin having a β-sheet conformation of at least 20%, at least 50%, or at least 80%.

6. The drug delivery platform of claim 1, wherein the hydrogel comprises silk fibroin having an α-helical and random coil conformation of at most 20%.

7. The drug delivery platform of claim 1, wherein the compound has the structure of formula II 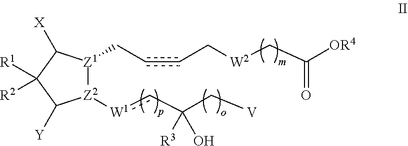
wherein each dashed line represents the presence or absence of a bond;
R¹, R² and R³ are each independently selected from H or C₁-C₆ alkyl;
R⁴ is H, C₁-C₆ alkyl, C₂-C₆ alkenyl, a pharmaceutically acceptable salt thereof, or a pharmaceutically acceptable amine salt thereof;
X and Y are each independently selected from H, OH, ═O, Cl, Br, I, or CF₃;
Z¹ and Z² are each independently selected from CH or N;
W¹ and W² are each independently selected from CH, CH₂, aryl or substituted aryl, heteroaryl, substituted heteroaryl;
m is 0 to 4;
p is 0 or 1;
o is 0 to 4; and
V is C₁-C₆ alkyl, aryl, substituted aryl, heteroaryl, or substituted heteroaryl.

8. The drug delivery platform of claim 1, wherein V is 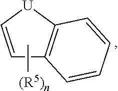
wherein U is C, N, O, or S; R⁵ is halogen, C₁-C₆ alkyl, or C₂-C₆alkenyl; and n is 0-7.

9. The drug delivery platform of claim 8, wherein U is S; R⁵ is F, Cl, Br, or I; and n is 1, 2, or 3.

10. The drug delivery platform of claim 1, wherein W² is thiophene.

11. The drug delivery platform of claim 1, wherein the compound has the structure of formula III 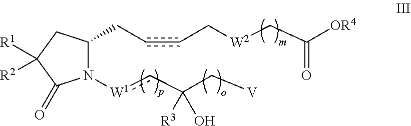
wherein each dashed line represents the presence or absence of a bond;
R¹, R² and R³ are each independently selected from H or C₁-C₆ alkyl;
R⁴ is H, C₁-C₆ alkyl, C₂-C₆ alkenyl, a pharmaceutically acceptable salt thereof, or a pharmaceutically acceptable amine salt thereof;
W¹ and W² are each independently selected from CH, CH₂, aryl or substituted aryl, heteroaryl, substituted heteroaryl;
m is 0 to 4;
o is 0 to 4;
p is 0 or 1; and
V is CH₃, aryl, aryl or substituted aryl, heteroaryl, substituted heteroaryl.

12. The drug delivery platform of claim 1, wherein the compound has the structure of formula IV 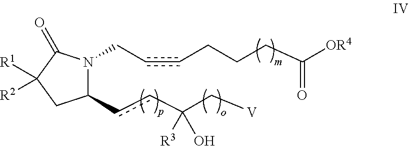
wherein each dashed line represents the presence or absence of a bond;
R¹, R² and R³ are each independently selected from H or C₁-C₆ linear alkyl;
R⁴ is H, C₁-C₆ alkyl, C₂-C₆ alkenyl, a pharmaceutically acceptable salt thereof, or a pharmaceutically acceptable amine salt thereof;
m is 0 to 4;
o is 0 to 4;
p is 0 or 1; and
V is CH₃, aryl, aryl or substituted aryl, heteroaryl, substituted heteroaryl.

13. The drug delivery platform of claim 1, wherein the compound has the structure of formula V 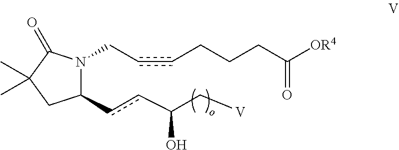
wherein each dashed line represents the presence or absence of a bond;
R⁴ is H, C₁-C₆ alkyl, C₂-C₆ alkenyl, a pharmaceutically acceptable salt thereof, or a pharmaceutically acceptable amine salt thereof;
o is 0 to 4; and
V is CH₃, aryl, aryl or substituted aryl, heteroaryl, substituted heteroaryl.

14. The drug delivery platform of claim 1, wherein the compound is 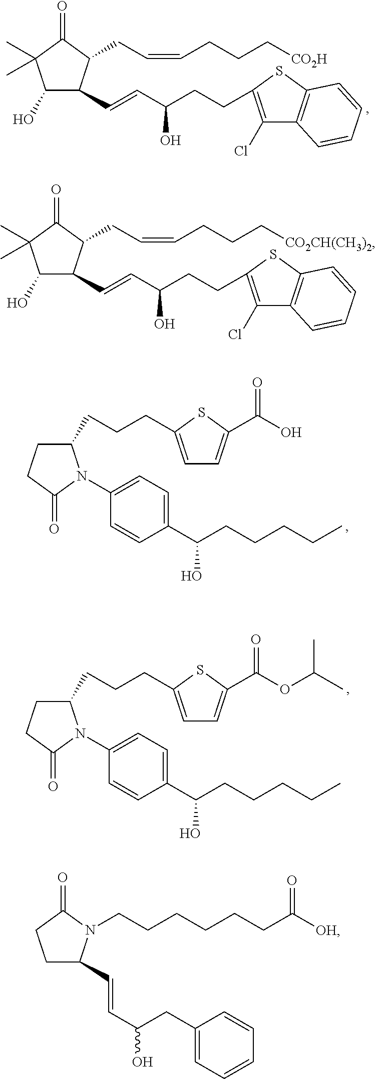
a pharmaceutically acceptable salt thereof, or a pharmaceutically acceptable amine salt thereof.

15. The drug delivery platform of claim 1, wherein the compound is 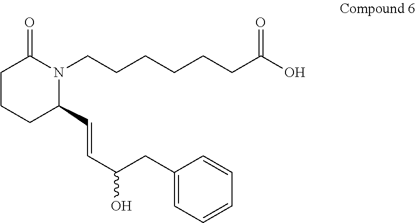
a pharmaceutically acceptable salt thereof, or a pharmaceutically acceptable amine salt thereof.

16. The drug delivery platform of claim 1, wherein the platform is an extended drug release platform or a sustained drug release platform.

17. The drug delivery platform of claim 16, wherein the extended drug release platform releases a pharmaceutically-active drug with substantially first order release kinetics over a period of at most 6 days after administration

18. The drug delivery platform of claim 16, wherein the sustained drug release platform releases a pharmaceutically-active drug with substantially first order release kinetics over a period of at least 45 days after administration.

19. The drug delivery platform of claim 1, wherein the platform is processed into a hydrogel, a sheet, a film, a porous material, a fiber, a thread, a solution, an oil, a lotion, a gel, an ointment, a cream, a slurry, a salve, a paste, a microsphere, a mesh, a mat, or a solid.

20. A method of treating a wound in an individual, the method comprising the step of administering a drug delivery platform of claim 1 to the wound of the individual, wherein administration promotes healing of the wound or reduced scaring, thereby treating the wound.
